(19) United States
(12) Patent Application Publication
(10) Pub. No.: US 2025/0387481 A1
(43) Pub. Date: Dec. 25, 2025

(54) PRE-TREATED T CELLS FOR USE AS A MEDICAMENT

(71) Applicant: STICHTING HET NEDERLANDS KANKER INSTITUUT-ANTONI VAN LEEUWENHOEK ZIEKENHUIS, Amsterdam (NL)

(72) Inventors: Jacobus Emiel VAN RHEENEN, Amsterdam (NL); Sabine Charlotte LINN, Amsterdam (NL); Claire Marie Cecile VENNIN, Amsterdam (NL)

(21) Appl. No.: 18/863,763

(22) PCT Filed: May 10, 2023
(86) PCT No.: PCT/NL2023/050254

§ 371 Date: Nov. 7, 2024

(30) Foreign Application Priority Data
May 10, 2022 (NL) 2031813
Apr. 17, 2023 (NL) 2034591

(51) Int. Cl. A61K 40/11; A61K 35/17; A61K 40/42; A61K 40/50; A61P 35/00; C12N 5/0783

(52) U.S. Cl. CPC A61K 40/11; A61K 35/17; A61K 40/42; A61K 40/50; A61P 35/00; C12N 5/0636; A61K 2239/49

(57) ABSTRACT

This invention pertains in general to therapy using pre-treated T cells and/or vesicles secreted by these T cells. In particular there is provided for the use of such pre-treated T cells and/or vesicles secreted by these T cells as a medicament. The pre-treated T cells and/or vesicles secreted by these T cells are useful in the treatment of various conditions including cancer. In particular the pre-treated T cells and/or vesicles secreted by these T cells are useful in therapy that is aimed at preventing adverse effects such as neuropathy that is normally induced by the use of antimitotic agents such as taxanes and vinca alkaloids. The invention also pertains to a method of producing the pre-treated T cells and/or vesicles secreted by these T cells of the invention.

### FIELD OF THE INVENTION

This invention pertains in general to therapy using pre-treated T cells and/or vesicles secreted by these T cells. In particular there is provided for the use of such pre-treated T cells and/or vesicles secreted by these T cells as a medicament. The pre-treated T cells and/or vesicles secreted by these T cells are useful in the treatment of various conditions including cancer. The pre-treated T cells and/or vesicles secreted by these T cells are useful in therapy that is aimed at preventing adverse effects such as neuropathy that is normally induced by the use of antimitotic agents such as taxanes and vinca alkaloids. The invention also pertains to a method of producing the pre-treated T cells and/or vesicles secreted by these T cells of the invention.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Antimitotic agents such as taxanes and vinca alkaloids are among the most widely employed chemotherapies for the treatment of cancer (Jordan, M. A. and L. Wilson, Nature Review Cancer, 2004. 4: p. 253-265)

At the same time, such antimitotic agents are also reported to have detrimental toxic side effects, some of which are irreversible, such as peripheral neuropathy, febrile neutropenia, seizure, and encephalopathy. For example, peripheral neuropathies induced by chemotherapy (CIPN) are recognized as being an increasingly frequent problem. Amongst the most frequent substances causing peripheral neuropathies are, next to platin compounds, antimitotic agents such as vinca alkaloids and taxanes. Currently, neither prophylaxis nor specific treatment is available, and only symptomatic treatment can be offered (Grisold et al. Neuro Oncol. 2012 September; 14(Suppl 4): iv45-iv54).

Although all patients treated with, for example, taxanes are exposed to these adverse side effects, not all patients experience clinical benefit. For example, a meta-analysis of long-term outcomes of 100,000 breast cancer patients revealed that the addition of taxanes to anthracycline regimens prolongs survival in only ˜3% of patients (Early Breast Cancer Trialists' Collaborative, G., et al., Lancet, 2012. 379(9814): p. 432-44).

In order to maximize the anti-tumor effects of antimitotic agents such as taxanes or vinca alkaloids, while limiting their toxicity, understanding the in vivo mode of action of these agents is critical. However, this in vivo mode of action still remains controversial. For example, for over four decades, the anti-cancer activity of taxanes, such as docetaxel and paclitaxel, has been attributed to their ability to trigger abnormal mitotic spindle formation by stabilizing microtubules, leading to delayed mitotic progression and cell death. This proposed mode of action is based mostly on in vitro studies (Jordan, M. A. and L. Wilson, Nature Review Cancer, 2004. 4: p. 253-265; Yvon, A. M. et al. Mol Biol 1999. Cell, 10(4): p. 947-59; Nogales, E., et al., Nature, 1995. 375(6530): p. 424-7; Schiff, P. B. et al. Nature, 1979. 277(5698): p. 665-7).

Conversely, data from both animal models and human cancer patients suggest that, for example, taxanes may induce apoptosis independent of blocking mitotic progression in the in vivo setting Milas, L., et al. Cancer Chemother Pharmacol, 1995. 35(4): p. 297-303; Janssen, A., et al., PLOS One, 2013. 8(5): p. e64029; Symmans, W. F., et al., Clin Cancer Res, 2000. 6(12): p. 4610-7; Milross, C. G., et al. J Natl Cancer Inst, 1996. 88(18): p. 1308-14). For instance, it has been reported that taxanes trigger apoptosis in cancer cells via perturbing mitosis progression in vitro, while taxane-mediated killing of the same cancer cells growing in a native in vivo environment was independent of such mitotic defects (Janssen, A., et al., PLOS One, 2013. 8(5): p. e64029). Similarly, analysis of human and mouse tumor tissues showed that taxanes treatment induced only a moderate increase in mitotic index, which did not correlate with tumor response to treatment (Symmans, W. F., et al., Clin Cancer Res, 2000. 6(12): p. 4610-7; Milross, C. G., et al. J Natl Cancer Inst, 1996. 88(18): p. 1308-14; Orth, J. D., et Cancer Res, al., 2011. 71(13): p. 4608-16; Maia, A. R. R., et al., Br J Cancer, 2018. 118(12): p. 1586-1595).

In order to maximize the therapeutic effects, for example anti-tumor effects, of antimitotic agents, such as taxanes or vinca alkaloids, there is a need for new treatment options. Such new treatments should be based on a better understanding of the in vivo mode of action of these antimitotic agents. In addition, also because the number of long-term cancer survivors increases, there is a need for treatments that take advantage of the mode of action/therapeutic effects of antimitotic agents such as taxanes or vinca alkaloids but that at the same time prevent or limit the exposure of the patient to the adverse side effects as the consequence of the use of these antimitotic agents, in particular adverse effects such as neuropathy that are induced by the treatment of a subject with antimitotic agents such as taxanes or vinca alkaloids.

In light of this, new (pharmaceutical) compositions, use thereof as a medicament or in therapy, in particular in the treatment of cancer and/or in treatment aimed at preventing or limiting adverse effects, such as neuropathy, induced by the use of antimitotic agents such as taxanes or vinca alkaloids, method for preparing such medicaments, and methods of treatment using such medicaments are highly desirable, but are not yet readily available. Accordingly, the technical problem underlying the present invention can been seen in the provision of such products, compositions, methods and uses for complying with any of the aforementioned needs. The technical problem is solved by the embodiments characterized in the claims and herein below.

### SUMMARY OF THE INVENTION

The inventors have discovered a new mode of action of antimitotic agent, such as a taxane or a vinca alkaloid, mediated cell killing in the in vivo setting. Using mouse- and patient-derived models of cancer, autologous organoid co-culture platforms and proteomic analyses, the inventors have uncovered that antimitotic agents, such as a taxane or a vinca alkaloid taxane, can directly activate T cells to specifically eradicate tumor cells. Mechanistically, present inventors demonstrate that antimitotic agents, such as a taxane or a vinca alkaloid, induce a release of cytotoxic extracellular vesicles by T cells that subsequently trigger tumor death and also reveal that T cells pre-treated with these antimitotic agent's ex vivo have an anti-tumor capacity once transplanted in vivo.

As embodied and broadly described herein, the present invention is directed to the surprising finding that T cells, for example isolated T cells, for example tumor naïve and tumor reactive isolated T cells (for example obtained from a subject, for example, a the subject intended to be treated, of T cells obtained by in vitro methods), treated outside the body of a subject (for example ex vivo or in vitro) with an antimitotic agent, such as a taxane or a vinca alkaloid, selectively kill cancer cells when administered to a subject. It was also surprisingly found that extracellular vesicles released from said treated T cells are able to kill cancer cells when administered to a subject. The (selective) killing of the cancer cells was found to be non-canonical and T cell receptor-independent.

The present invention thus allows for a new and effective therapeutic approach, based on T cells pre-treated with an antimitotic agent, such as a taxane or a vinca alkaloid. The T cells are pre-treated with an antimitotic agent, such as a taxane or a vinca alkaloid. After the treatment with the antimitotic agent the T cells and/or extracellular vesicles released from said treated T cells can be used as a medicament and can be administered to a subject in need thereof.

The treatment of the T cells outside the subject and the subsequent transfer of the T cells and/or extracellular vesicles released from said treated T cells to the subject importantly also avoids toxicity that is normally associated with systemic treatment with antimitotic agents. The invention thus allows for a therapy that takes advantage of the beneficial effects of an antimitotic agent, such as a taxane or a vinca alkaloid, on T cells, as surprisingly found and described herein, without the requirement of systemic treatment with the antimitotic agent. Consequently, the new therapy as disclosed can prevent neuropathy, preferably peripheral neuropathy, for example vinca alkaloid-induced neuropathy and/or taxane-induced neuropathy in a subject and that is a common adverse effect of systemic treatment with such antimitotic agent. It was found that healthy cells, e.g., epithelial cells, are not killed. This is because the invention allows to reduce systemic exposure to such antimitotic agents while at the same time providing therapeutic effect of such antimitotic agents to the subject by treating T cells with such antimitotic agents before providing these to the subject.

The pre-treated T cells are in particular useful as a medicament in the treatment of various conditions that would normally involve treatment with an antimitotic agent an antimitotic agent, such as a taxane or a vinca alkaloid. In particular the pre-treated T cells can be used in the treatment of cancer and/or in the prevention of neuropathy, for example vinca alkaloid-induced neuropathy and/or taxane-induced neuropathy (i.e., can be used in a treatment of a condition in a subject that would benefit from the use of an antimitotic agent, such as a taxane or a vinca alkaloid, without inducing adverse effects, such as neuropathy, associated with the systemic use of such antimitotic agent)./pct1

Therefore, according to the invention there is provided for T cells obtainable by contacting isolated T cells with at least one antimitotic agent, preferably selected from the groups consisting of a taxane and a vinca alkaloid, and/or extracellular vesicles released from said T cells, for use as a medicament. The T cells according to the invention and/or extracellular vesicles released from said T cells can be prepared by contacting the T cells with at least one antimitotic agent preferably selected from the groups consisting of a taxane and a vinca alkaloid, preferably with at least one taxane. The thus obtained T cells and/or extracellular vesicles released from said T cells can be used as a medicament and can be transferred to or administered to a subject in need thereof.

The T cells are treated with the antimitotic agent, such as a taxane or a vinca alkaloid, outside the body (e.g., ex vivo or in vitro). For example, T cells may first be isolated from a sample obtained from a subject, treated with an antimitotic agent, such as a taxane or a vinca alkaloid, and after the treatment, be re-introduced to the subject. Alternative T cells from another subject or available from the shelf may be treated as disclosed herein and be introduced to the subject.

In some embodiments, the T cells comprise CD4+ T cells and/or CD8+ T cells and/or the T cells are autologous T cells, allogenic T cells or syngeneic T cells. The T cells may also be naïve T cells (i.e., T cells that have matured but have not yet encountered its corresponding antigen e.g., as presented on the cell surface of a cancer cells) or tumor-reactive T cells. The T cells may be immature T cells or mature T cells.

In some embodiments, the antimitotic agent is a vinca alkaloid, preferably selected from the group consisting of vinblastine, vinorelbine, vincristine, vindesine and vinflunine. Preferably the vinca alkaloid is vincristine or vinblastine.

In some embodiments, the antimitotic agent in a taxane, preferably selected from the group consisting of paclitaxel and docetaxel. Other useful examples of taxanes that can be used according to the invention include nanoparticle albumin-bound (“nab-”) taxanes such as nab-paclitaxel and nab-docetaxel.

In some embodiments, a combination of vinca alkaloids, taxanes, or vinca alkaloids and taxanes are used to treat the T cells.

In some embodiments, the treated T cells and/or the extracellular vesicles released from said T cells are used as a medicament in the treatment of a condition that would normally benefit from or is treated with an antimitotic agent, such as a taxane or a vinca alkaloid, and wherein said antimitotic agent is administered to the subject (e.g., systemically or locally).

In some embodiments, the treated T cells and/or the extracellular vesicles released from said T cells are used as a medicament in the treatment of a subject that cannot or is not willing to be treated with an antimitotic agent, such as a taxane or a vinca alkaloid.

In some embodiments, the treated T cells and/or the extracellular vesicles released from said T cells are used as a medicament in the treatment of a subject suffering from cancer.

In some embodiments, the treated T cells and/or the extracellular vesicles released from said T cells are used as a medicament in the prevention of neuropathy, preferably peripheral neuropathy, vinca alkaloid-induced neuropathy and/or taxane-induced neuropathy in a subject.

In some embodiments, the treated T cells and/or the extracellular vesicles released from said T cells are used as a medicament in therapy wherein the therapy is aimed at preventing neuropathy, preferably peripheral neuropathy, vinca alkaloid-induced neuropathy and/or taxane-induced neuropathy in a subject. In such embodiments, the treated T cells and/or the extracellular vesicles released from said T cells may be used in the treatment of a condition that would benefit from treatment with an antimitotic agent, such as a vinca alkaloid or a taxane, and reduce or prevent adverse effects, such as neuropathy, in particular vinca alkaloid-induced neuropathy and/or taxane-induced neuropathy by not requiring or by limiting the administration of such antimitotic agent to the subject.

In some embodiments, there is provided for a method of producing treated T cells, and/or extracellular vesicles released from said T cells, wherein the method comprises the steps of
(a) providing isolated T cells;
(b) contacting the provided T cells with at least one antimitotic agent, preferably selected from the groups consisting of a taxane and a vinca alkaloid, preferably in an aqueous medium; and
(c) obtaining the T cells contacted with the at least one antimitotic agent, and obtaining the extracellular vesicles released from said T cells, to provide for the treated T cells and/or extracellular vesicles released from said T cells. In some embodiments the method is for preparing a medicament.

In some embodiments, there is provided for a method of producing extracellular vesicles released from treated T cells, wherein the method comprises the steps of
(a) providing isolated T cells;
(b) contacting the provided T cells with at least one antimitotic agent, preferably selected from the groups consisting of a taxane and a vinca alkaloid, preferably in an aqueous medium; and
(c) obtaining the T cells contacted with at least one antimitotic agent, and obtaining the extracellular vesicles released from said T cells, to provide for the extracellular vesicles released from said T cells. In some embodiments the method is for preparing a medicament.

In some embodiments, there is provided for a method of producing treated T cells, and/or extracellular vesicles released from said T cells, wherein the method comprises the steps of
(a) providing isolated T cells;
(b) contacting the provided T cells with at least one antimitotic agent, consisting of a vinca alkaloid, preferably in an aqueous medium; and
(c) obtaining the T cells contacted with at least one antimitotic agent, and/or obtaining the extracellular vesicles released from said T cells, to provide for the treated T cells and/or extracellular vesicles released from said T cells. In some embodiments the method is for preparing a medicament.

The thus obtained T cells and/or extracellular vesicles released from said T cells may be used as a medicament and may be administered to a subject in need thereof. The thus obtained T cells and/or extracellular vesicles released from said T cells may also be stored, for example, may be stored at low temperature, e.g., below 10 degrees Celsius, in the form of a pharmaceutical acceptable composition, and subsequent be used as a medicament and administered to a subject in need thereof. In some embodiments there is provided for a pharmaceutical composition comprising treated T cells according to the invention and/or extracellular vesicles released from said T cells and at least one, two, three, four, five or six, or any combination of any two, three, four, five thereof, selected from the group consisting of a isotonic solution of electrolytes (preferably plasma-lyte A), dextrose, sodium chloride, a dextran (preferably dextran 40), DMSO and serum albumin, for example human serum albumin.

In some embodiment there is provided for a method of treatment of a subject in need thereof wherein the method comprises administering to the subject T cells obtainable by contacting isolated T cells with at least one antimitotic agent, preferably selected from the groups consisting of a taxane and a vinca alkaloid, and/or administering to the subject extracellular vesicles released from said T cells.

This and other aspect will be apparent to the skilled person from the disclosure, and in particular from the examples described herein.

### BRIEF DESCRIPTION OF THE DRAWINGS

Embodiments of the invention are further described hereinafter with reference to the accompanying drawings, in which:

FIG. 1: T cells mediate the in vivo anti-tumor effects of docetaxel. A. Schematic representation of experimental design and treatment timeline in the KB1P tumor model. B, C. KB1P tumor volume over the course of treatment with AC (B) or with AC+T (C) in the presence (IgG control) or absence of T cells (anti-CD4 Ab+anti-CD8 Ab). Dotted line indicates the threshold of tumor detection. Also see FIG. 7. D. Schematic representation of experimental design for isolation of intratumoral T cells followed by co-culture with organoids. Following in vivo treatment with a vehicle control, AC or AC+T, KB1P tumors were excised and intratumoral CD4 and CD8 T cells were isolated by FACS and co-cultured with chemotherapy-naïve KB1P organoids. Also see FIG. 8. E. MTT assay assessment of KB1P organoid survival following co-culture with CD4 and CD8 T cells isolated from KB1P tumors treated with a vehicle control, AC or AC+T. n=3 biological repeats with three technical replicates per repeat. F. Representative images and quantification of immunofluorescence staining for cleaved-caspase 3 (CC3) in KB1P organoids following co-culture with CD4 and CD8 T cells isolated from KB1P tumors treated with a vehicle control, AC or AC+T. Scale bar: 50 μm. n=3 biological repeats with three technical replicates per repeat. Data are presented as mean with standard-error of the mean (SEM). G. Schematic representation of experimental design and treatment timeline. H. Kaplan-Meier survival curve for mice bearing KB1P tumors treated with a vehicle control or docetaxel upon administration of an IgG control or of depleting antibodies against CD4 T cells only, CD8 T cells only or CD4 and CD8 T cells.

FIG. 2: Docetaxel directly increases T cell cytotoxic activity. A. Schematic representation of the isolation of splenic T cells and co-culture with tumor organoids. Also see FIG. 10. B. MTT assay assessment of KB1P organoid survival following co-culture with CD4 or CD8 T cells, pre-treated with a vehicle control or with docetaxel. C. Representative images and quantification of immunofluorescent staining for cleaved-caspase 3 in KB1P organoids following co-culture with either CD4 or CD8 T cells pre-treated with a vehicle control or with docetaxel. Scale bar: 100 μm. D. MTT assay assessment of KB1P organoid survival, E. representative images and quantification of immunofluorescent cleaved-caspase 3 staining in KB1P organoids following co-culture with CD4 T cells pre-treated with a vehicle control or with docetaxel, in the presence or absence of an LCK inhibitor (LCKi). Scale bar: 100 μm. F. MTT assay assessment of KB1P organoid survival, G. representative images and quantification of immunofluorescent cleaved-caspase 3 staining in KB1P organoids following co-culture with CD8 T cells pre-treated with a vehicle control or with docetaxel, in the presence or absence of an LCK inhibitor (LCKi). Scale bar: 100 μm. H and I. MTT assay assessment of MMTV-PyMT organoid survival (H) and representative images and quantification of immunofluorescent cleaved-caspase 3 staining (I) in MMTV-PyMT organoids following co-culture with CD4 T cells pre-treated with a vehicle control or with docetaxel, in the presence or absence of an LCK inhibitor (LCKi). Scale bar: 100 μm. J. and K. MTT assay assessment of MMTV-PyMT organoid survival (J) and representative images and quantification of immunofluorescent cleaved-caspase 3 staining (K) in MMTV-PyMT organoids following co-culture with CD8 T cells pre-treated with a vehicle control or with docetaxel, in the presence or absence of an LCK inhibitor (LCKi). Scale bar: 100 μm. n=3 biological repeats with three technical replicates per repeat. Data are presented as mean with SEM. Also see FIGS. 11 and 12. FIG. 2 (L) quantification of immunofluorescent staining for cleaved-caspase 3 in MMTV-PyMT organoids expressing OVA and co-cultured with T cells isolated from the spleen of OT I mice, upon treatment with the LCKi. p-values were determined using a two-tailed paired t test.

FIG. 3: Docetaxel increases the anti-tumor activity of human T cells. A. Schematic representation of the generation of breast cancer patient-derived organoids and co-culture with Jurkat T cells. B and C. Quantification (B) and representative images of cleaved-caspase 3 immunofluorescent staining (C) in 7 breast cancer patient-derived organoid lines co-cultured with control or with docetaxel-treated Jurkat T cells. Scale bar=100 μm. n=3 biological repeats with 1 technical replicate per repeat. D. Schematic representation of the generation of tumor-reactive human T cells and autologous tumor organoids derived from a lung cancer patient. T cells were treated with docetaxel in vitro and co-cultured with matched tumor organoids. Also see FIG. 14. E. Representative images of co-cultures of human lung cancer organoids with matched tumor-reactive T cells pre-treated with a vehicle control or with docetaxel, at day 0 and day 1. Scale bar=200 μm. F. Quantification of luciferase reporter activity in human lung tumor organoids after co-culture with tumor-reactive T cells pre-treated with a vehicle control or with docetaxel. n=3 biological replicates with three technical repeats per replicate. Data are presented as mean with SEM. Images were adapted from Servier Medical. G-I. Quantification of cleaved-caspase 3 immunofluorescent staining in 3 patient-derived cancer organoids upon a 72 h treatment with the EV and SF isolated from the conditioned media generated by tumor reactive T cells treated with a vehicle control or docetaxel. n=3 biological replicates, data are presented as mean+/−SEM. J and K. Quantification of cleaved-caspase 3 immunofluorescence staining in patient-derived lung healthy organoids (patient 1) and colon healthy organoids (patient 3) upon treatment with the EVs and the soluble fraction generated by tumor-reactive T cells treated with a vehicle control or with docetaxel. n=3 biological repeats.

FIG. 4: Docetaxel induces release of cytotoxic extracellular vesicles by T cells to eliminate tumor organoids. A and B. MTT assay assessment of KB1P organoid survival (A) and representative images and quantification of immunofluorescent staining for cleaved-caspase 3 (B) in KB1P organoids upon treatment with conditioned media generated by CD4 T cells or CD8 T cells pre-treated with a vehicle control or with docetaxel. C and D. MTT assay assessment of MMTV-PyMT organoid survival (C) and representative images and quantification of immunofluorescent staining for cleaved-caspase 3 (D) in MMTV-PyMT organoids upon treatment with conditioned media generated by CD4 or CD8 T cells pre-treated with a vehicle control or with docetaxel. For A-D, n=3 biological repeats with three technical replicates per repeat. Also see FIG. 15A, B. E. Volcano plot (left) representing extracellular peptides differently represented in the secretome of T cells treated with a vehicle control (grey) versus docetaxel (purple) and quantification of percentage of proteins identified as EV in the conditioned media of docetaxel-treated T cells (right bar graph). n=3 biological repeats with one technical replicate per repeat. Also see FIG. 15C, D. F. Electron microscopy images of EVs isolated from conditioned media generated by control T cells or docetaxel-treated T cells. G. NanoSight particle analysis displaying the size distribution of EVs isolated from conditioned media generated by control T cells or docetaxel-treated T cells. Also see FIG. 16. For F and G, n=1 biological repeat with 1 technical replicate. H and I. Representative images and quantification of cleaved-caspase 3 immunofluorescent staining in KB1P organoids (H) and in MMTV-PyMT organoids (I) upon a 72 h treatment with EVs isolated from conditioned media generated by control T cells or docetaxel-treated T cells. J and K. Representative images and quantification of cleaved-caspase 3 immunofluorescent staining in KB1P organoids (J) and in MMTV-PyMT organoids (K) upon a 72 h treatment with the soluble fraction (SF) isolated from conditioned media generated by control T cells or docetaxel-treated T cells. L. Volcano plot representing the peptides detected in the EV fractions released by control (left) versus docetaxel-treated (right) T cells. M. Quantification of cleaved-caspase 3 immunofluorescence staining in MDA-MB-231 cells upon exposure to increasing doses of EVs released by docetaxel-treated Jurkat T cells. For H-K, L, M n=3 biological repeats with one technical replicate per repeat. Scale bar=200 μm.

FIG. 5: T cells pre-treated with docetaxel ex vivo can eliminate tumors in vivo. A and B. Representative images and quantification of cleaved-caspase 3 immunofluorescent staining (A) and survival analysis using MTT (B) in MECs after co-culture with CD4 or CD8 T cells pre-treated with a vehicle control or with docetaxel. n=3 biological repeats with three technical replicates per repeat. Data were compared to results obtained for tumor organoids depicted in FIG. 2. Scale bar=100 μm. Also see FIG. 17. D. Representative images of mTmG-MECs, KB1P organoids and MMTV-PyMT organoids and quantification of percentage of cells containing EV-PKH67 1 h after incubation with EV-PKH67. n=3 biological repeats with one technical replicate per repeat. Scale bar=50 μm. E. Schematic representation of experimental set up for transfer of T cells pre-treated with docetaxel into tumor-bearing mice. F and G. Tumor growth in mice bearing KB1P tumors (E) or MMTV-PyMT tumors (G) and transplanted with CD4 T cells pre-treated in vitro with a vehicle control or with docetaxel. H and I. Kaplan-Meier analysis of survival from time of T cell transfer in mice bearing KB1P tumors (H) or MMTV-PyMT tumors (I) transplanted with CD4 T cells pre-treated in vitro with a vehicle control or with docetaxel. (J) quantification of cleaved-caspase 3 immunofluorescent staining in MMTV-PyMT organoids expressing OVA and co-cultured with T cells isolated from the spleen of OT I mice upon pre-treatment with a vehicle control or docetaxel (n=3 biological repeats).

FIG. 6: Docetaxel does not induce mitotic defects in vivo. A. Schematic representation of treatment timeline with control or docetaxel in KB1P or MMTV-943 PyMT tumor models. B and C. KB1P tumor volume (B) and MMTV-PyMT tumor volume (C) during treatment with a vehicle control or with docetaxel. D and E. Representative H&E images (D) and quantification (E) of cells with a mitotic plate in KB1P tumors and in MMTV-PyMT tumors treated with control or with docetaxel. n=4 mice per treatment group. Scale bar=100 μm.

FIG. 7: Depleting antibodies efficiently ablate CD4 and CD8 T cells from KB1P tumors. A. Representative images and quantification of CD4 positive cells in KB1P tumors treated with an IgG control or with a CD4-depleting antibody. B. Representative images and quantification of CD8 positive cells in KB1P tumors treated with an IgG control or with a CD8-depleting antibody. Tumors were isolated from mice treated as depicted in FIG. 1G at endpoint. n=4 mice per treatment group. Scale bar=100 μm.

FIG. 8: FACS quantification of intratumoral T cells in KB1P and MMTV-PyMT tumors upon treatment with docetaxel and role of T cells in driving MMTV-PyMT tumors response to docetaxel. A. Gating strategy for FACS isolation of intratumoral T cells from KB1P or MMTV-PyMT tumors in ROSAmTmG female mice. B. Quantification of T cell infiltration in KB1P tumors treated with a vehicle control, AC, or AC+T. C. Quantification of T cell infiltration into MMTV-PyMT tumors treated with a vehicle control, AC, or AC+T. D. MMTV-PyMT organoid viability, assessed by MTT, following co-culture with intratumoral T cells isolated from MMTV-PyMT tumors treated in vivo with a vehicle control, AC or AC+T. E and F. Representative images and quantification of CD4 (E) and CD8 (F) immunohistochemical staining in KB1P tumors treated with a vehicle control or with docetaxel. Unless stated otherwise on the figure, scale bar=200 μm. G. Kaplan-Meier survival analysis in mice bearing MMTV-PyMT tumors treated with a vehicle control or with docetaxel upon administration of an IgG control or of depleting antibodies against CD4 T cells only, CD8 T cells only or CD4 and CD8 T cells. Individual data points represent measurements from individual tumors. Data are presented as mean with SEM.

FIG. 9: AC+T treatment does not render KB1P organoids more sensitive to T cell killing. A. Schematic representation of tumor organoids co-cultured with intratumoral T cells isolated from control KB1P tumors and from KB1P organoids pre-treated with a vehicle control, AC or AC+T, followed by co-culture. B. MTT assay assessment of KB1P organoid survival following co-culture with intratumoral control T cells. n=3 biological replicates with three technical repeats.

FIG. 10: FACS gating strategy for isolation of splenic T cells and optimization of treatment of T cells with docetaxel. A. Gating strategy for FACS isolation of splenic T cells. B. MTT assay assessment of survival of T cells treated with increasing doses of docetaxel. n=3 biological repeats with three technical replicates. Data are presented as mean with SEM. C. Quantification of microtubule and F-actin. n=60 for control and n=62 for docetaxel-treated cells pooled from 3 biological repeats.

FIG. 11: Taxane-specific increase of T cell-mediated killing of tumor organoids. A and B. MTT assay assessment of survival of T cells treated with increasing doses of paclitaxel (A) or carboplatin (B). C. MTT assay assessment of KB1P organoids survival upon co-culture with CD4 and CD8 T cells pre-treated with a vehicle control or with paclitaxel. D. MTT assay assessment of KB1P organoids survival upon co-culture with CD4 and CD8 T cells pre-treated with a vehicle control or with carboplatin. E. MTT assay assessment of MMTV-PyMT organoids survival upon co-culture with CD4 and CD8 T cells pre-treated with a vehicle control or with paclitaxel. F. MTT assay assessment of MMTV-PyMT organoids survival upon co-culture with CD4 and CD8 T cells pre-treated with a vehicle control or with carboplatin. n=3 biological repeats with three technical replicates per repeat. Data are presented as mean with SEM.

FIG. 12: In vitro killing of KB1P organoids by T cells pre-treated with docetaxel is not mediated by direct T cell: organoid contact. A. Representative images of KB1P organoids and quantification of changes in organoid perimeter during co-culturing with CD4 T cells isolated from the spleen of healthy ROSAmTmG female mice and pre-treated with vehicle or with docetaxel. B. Representative images of KB1P organoids and quantification of changes in organoid perimeter during co-culturing with CD8 T cells isolated from the spleen of healthy ROSAmTmG mice and pre-treated with vehicle or with docetaxel. C. Representative images of T cells isolated from the spleen of ROSAmTmG accumulating on the edge of the BME drop, failing to infiltrate deep inside the BME drop. Dotted line depicts the edge of the BME drop. D. Quantification of the number of CD4 T cells (left) and CD8 T cells (right) on the edges of the BME drop (dotted line) and inside the BME drop (solid line) during the duration of the co-culture experiment, after pre-treatment of T cells with vehicle or with docetaxel. E. Representative images of cleaved-caspase 3 staining and quantification in KB1P organoids co-cultured with T cells isolated from NOD-SCID-IL2rg-/-(NSG) mice and pre-treated with vehicle or with docetaxel. n=3 biological repeats with three technical replicates per repeat. Data are presented as mean with SEM. Scale bar=100 μm.

FIG. 13: Docetaxel induces Jurkat T cell-mediated killing in human cancer cell lines. A. MTT assessment of Jurkat T cell survival following a 72 h treatment with control or 5 nmol/L docetaxel. B-J. Representative images and quantification of cleaved-caspase 3 staining upon co-culture with control Jurkat T cells or docetaxel-treated Jurkat T cells in human B and C breast cancer cell lines, D melanoma cell line, E lung adenocarcinoma cell line, F cholangiocarcinoma cell line, G and H hepatocellular carcinoma cell lines, I rectal carcinoma cell line and J glioma cell line. n=3 biological repeats with one technical replicate per repeat. Data are presented as mean with SEM. Scale bar=100 μm.

FIG. 14: Docetaxel does not increase T cell tumor reactivity in a model of human lung cancer. A. Gating strategy for FACS isolation of CD4 and CD8 T cells from lung cancer patient-derived PBMCs. B. Quantification of live cells in PBMC derived from a lung cancer patient (Cattaneo, C. M., et al., Nat Protoc, 2020 15(1): p. 15-39; Dijkstra, K. K., et al. Cell, 2018. 174(6): p. 1586-1598 e12) and treated with a control vehicle or with docetaxel 10 nmol/L for 48 hr. n=3 with 1 technical replicate. C and D. Gating strategy (C) and quantification (D) of CD137 expression on tumor-reactive CD4 and CD8 T cells alone, in the presence of cancer organoids and upon treatment with PMA and ionomycin. T cells were pre-treated with a control vehicle or with 10 nmol/L docetaxel for 48 h. CD137 is used as a readout of TCR engagement. n=3 biological repeats with one technical replicate per repeat. E. Quantification of IFNγ secretion by T cells upon a 48 h-treatment with a control vehicle or with 10 nmol/L docetaxel, as a readout of cytotoxic activity. n=1 or 2 with one technical replicate per repeat. F. CD107 expression and G. PD1 expression on tumor-reactive CD4 and CD8 T cells alone, cultured in the presence of cancer organoids and upon treatment with PMA and ionomycin.

FIG. 15: Docetaxel IC50 for KB1P and MMTV-PyMT organoid lines and the absence of fetal bovine serum (FBS) does not influence on docetaxel-driven activation of T cells in vitro. A and B. MTT measurements of KB1P (A) and MMTV-PyMT (B) organoid survival upon treatment with increasing doses of docetaxel for 72 h. C. MTT assay assessment of survival of KB1P organoids following co-culture with CD4 or CD8 T cells, pre-treated with a vehicle control or with docetaxel, in the presence (left) or absence (right) or FBS. D. MTT assay assessment of survival of MMTV-PyMT organoids following co-culture with CD4 or CD8 T cells, pre-treated with a vehicle control or with docetaxel, in the presence (left) or absence (right) or FBS. E. Quantification of cleaved-caspase 3 positive cells in Hela WT and Hela ABCB1+ cells following co-culture with Jurkat T cells, pre-treated with a vehicle control or with 10 nmol/L paclitaxel for 72 h. F. MTT assay assessment of survival of Hela WT and Hela ABCB1+ cells following co-culture with Jurkat T cells, pre-treated with a vehicle control or with 10 nmol/L paclitaxel for 72 h. n=3 biological repeats with three technical replicates per repeat. Data are presented as mean with SEM.

FIG. 16: Schematic representation of isolation of the EV and soluble fractions from conditioned media generated by T cells.

FIG. 17: Docetaxel does not increase T cell reactivity against healthy human cells. A. Representative images and quantification of cleaved-caspase 3 immunofluorescent staining in human milk-derived organoids co-cultured with control Jurkat T cells or with docetaxel-treated Jurkat T cells. n=2 or 3 biological repeats with 1 technical replicate per repeat. Scale bar=100 μm. B and C. Representative images and quantification of cleaved-caspase 3 staining in (B) RPE and (C) HEK293T non-cancer cell lines upon co-culture with control or docetaxel-treated Jurkat T cells. n=3 biological repeats with one technical replicate per repeat. D. Representative images and diameter at day 1 of healthy lung organoids derived from the same patient as in FIG. 3D and co-cultured with tumor-reactive T cells pre-treated with a control vehicle or with docetaxel. n=3 biological repeats with one technical replicate per repeat. Data are presented as mean with SEM. Scale bar=100 μm.

FIG. 18: Cancer survival (assessed by MTT assay) following co-culture with human Jurkat T cells pretreated with control, vinblastine, or colchicine. Data are shown for three different cell lines derived from human cancer, namely A375 (melanoma), EGL1 (hepatobiliary cancer) and MDA-MB-231 (breast cancer). B. Organoid apoptosis (determined by cleaved caspase 3 staining) in breast cancer mouse KB1P organoids following co-culture with mouse splenic T cells pretreated with control, vinblastine, or colchicine. Data are presented as mean with SEM. N=3 biological repeats per repeat. Vinblastine, an exemplary vinca alkaloid, induces T cell mediated cancer cell killing.

### DESCRIPTION

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular forms “a,” “an” and “the” include plural referents unless the context clearly dictates otherwise. For example, a method for administrating a pharmaceutical agent includes the administrating of a plurality of molecules (e.g., 10's, 100's, 1000's, 10's of thousands, 100's of thousands, millions, or more molecules).

As used herein, “about” and “approximately”, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed invention. Unless otherwise clear from context, all numerical values provided herein include numerical values modified by the term “about.”

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps, or components. It also encompasses the more limiting “to consist of”.

As used herein, “conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology, and related fields are well-known to those of skill in the art and are discussed, in various handbooks and literature references.

As used herein, “exemplary” or “for example” means “serving as an example, instance, or illustration,” and should not be construed as excluding other configurations, including those disclosed herein.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range including both integers and non-integers. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 etc. This applies regardless of the breadth of the range.

As used herein, “cancer” refers to the physiological condition in mammals that is typically characterized by unregulated cell growth. The terms “cancer,” “neoplasm,” and “tumor,” are often used interchangeably to describe cells that have undergone a malignant transformation that makes them pathological to the host organism. Primary cancer cells can be distinguished from non-cancerous cells by techniques known to the skilled person. A cancer cell, as used herein, includes not only primary cancer cells, but also cancer cells derived from such primary cancer cell, including metastasized (secondary) cancer cells, and cell lines derived from cancer cells. Examples include solid tumors and non-solid tumors or blood tumors. Treatment of a cancer in a subject includes the treatment of a tumor in the subject.

Drugs, therapeutic agents, medicaments, and pharmaceutical compositions according to the present invention may be formulated for administration by a number of routes, including but not limited to, parenteral, intravenous, intra-arterial, intramuscular, intratumoral and oral. Drugs, therapeutic agents, medicaments, and compositions may be formulated in fluid or solid form. Fluid formulations may be formulated for administration by injection to a selected region of the human or animal body. Preferably the treated T cells according to the invention, when used as a medicament, are formulated for administration in fluid formulations, preferably suitable for injection, for example for intravenous, intra-arterial, intramuscular, or intratumoral delivery or injection.

As used herein, the term “effective amount” includes a dosage sufficient to produce a desired result with respect to the indicated disorder, condition, or mental state. The desired result may comprise a subjective or objective improvement in the recipient of the dosage. For example, an effective amount of T cells contacted with an antimitotic agent, for example a taxane (e.g., docetaxel), includes an amount sufficient to alleviate the signs, symptoms, or causes of cancer in a subject.

As used herein, the term “pharmaceutical composition” refers to a composition formulated in pharmaceutically-acceptable or physiologically-acceptable compositions for administration to a cell or subject. The compositions of the invention may be administered in combination with other agents as well, provided that the additional agents do not adversely affect the ability of the composition to deliver the intended therapy. The pharmaceutical composition often comprises, in addition to a pharmaceutical active agent, one or more pharmaceutical acceptable carriers (or excipients).

As used herein, “sample” when referring to biological material, in particular to T cells to be treated according to the invention, means a sample that has been removed from the subject; thus, none of the testing methods described herein are performed in or on the subject.

As used herein, a “subject” is to indicate the organism to be treated, for example, to which administration is contemplated. The subject may be any subject in accordance with the present invention, including, but not limited to humans, males, females, infants, children, adolescents, adults, young adults, middle-aged adults, or senior adults and/or other primates or mammals. Preferably the subject is a human patient. In some embodiments, the subject may have been diagnosed with a cancer, or be suspected of having a cancer.

As used herein, a “T-cell” can be selected from, for example, the group consisting of inflammatory T-lymphocytes, cytotoxic T-lymphocytes, regulatory T-lymphocytes, or helper T-lymphocytes. In another embodiment, said cell can be derived from the group consisting of CD4+ T-lymphocytes and CD8+ T-lymphocytes. They can be extracted from blood or derived from stem cells. T-cells can be obtained from a number of non-limiting sources, including peripheral blood mononuclear cells, bone marrow, lymph node tissue, cord blood, and tumors. In certain embodiments of the present invention, any T cell line available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics.

As used herein, “treatment”, “treating”, “palliating”, “alleviating” and “ameliorating” in the context of a subject to be treated, all refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. As used herein, “prevention” and “preventing” refers to an approach for reducing in part or in full the change of developing adverse effects, for example normally associated with the use of a particular drug or agent. Within the context of the current invention, for example, neuropathy, in particular neuropathy that is associated with the (systemic) use of an antimitotic agent, such as a taxane or a vinca alkaloid, can be prevented or circumvented by use of the treated T cells of the inventions as a medicament. Because with the invention the (systemic) use of an antimitotic agent, such as a taxane or a vinca alkaloid, can be prevented or be reduced, the use of the treated T cells of the invention thus prevents neuropathy, in particular taxane-induced neuropathy and/or vinca alkaloid-induced neuropathy. A diagnosis of neuropathy in the subject is not required.

### Detailed Description

The invention is defined herein, and in particular in the accompanying claims. Subject-matter which is not encompassed by the scope of the claims does not form part of the present claimed invention.

It is contemplated that any method, use, or composition described herein can be implemented with respect to any other method, use or composition described herein. Embodiments or preferences discussed in the context of methods, use and/or compositions of the invention may likewise be employed with respect to any other method, use or composition described herein. Thus, an embodiment or preference pertaining to one method, use or composition may be applied to other methods, uses and compositions of the invention as well.

Any references in the description to methods of treatment refer to the compounds, pharmaceutical compositions, and medicaments of the present invention for use in a method for treatment of the human (or animal) body by therapy.

As embodied and broadly described herein, the present invention is directed to the surprising finding that T cells that are treated with an antimitotic agent, in particular treated with a taxane or a vinca alkaloid selectively kill cancer cells when subsequently administered to a subject. It was also surprisingly found that extracellular vesicles released from said treated T cells are able to selectively kill cancer cells when administered to a subject. The invention thus allows for a new and effective therapeutic approach, based on T cells pre-treated with an antimitotic agent, in particular a taxane or a vinca alkaloid. It was found that T cells that are treated with an antimitotic agent (and EV obtained therefrom), in particular treated with a taxane or a vinca alkaloid selectively kill cancer cells in a TCR independent manner; i.e., provide for TCR-independent T cell cytotoxicity. The skilled person understand that such TCR-independent T cell cytotoxicity provided by the current invention may be present in addition to or independent of TCR-dependent T cell cytotoxicity, for example, of the same T cell.

Therefore, according to an aspect of the invention, there is provided for T cells obtainable by (or obtained by) contacting isolated T cells with at least one antimitotic agent, preferably selected from the groups consisting of a taxane and a vinca alkaloid, and/or extracellular vesicles released from said T cells, for use as a medicament.

There is also provided for T cells, obtainable by (or obtained by) contacting isolated T cells with at least one antimitotic agent, preferably selected from the groups consisting of a taxane and a vinca alkaloid, and/or extracellular vesicles released from said T cells, for use in therapy.

There is also provided for isolated T cells that have been contacted with at least one antimitotic agent, preferably selected from the groups consisting of a taxane and a vinca alkaloid, and/or extracellular vesicles released from said T cells, for use as a medicament.

T cells that can be obtained or have been obtained by treating T cells with at least one antimitotic agent, preferably selected from the groups consisting of a taxane and a vinca alkaloid, are herein also referred to as “treated T cells” or “pre-treated T cells”. The treated T cells and/or the extracellular vesicles released from said T cells are, as a medicament, preferably present ex vivo or in vitro, i.e., outside the body, prior to transfer of the treated T cells and/or the extracellular vesicles released from said T cells to the intended subject. Preferably the treated T cells and/or the extracellular vesicles released from said T cells are in the form of a pharmaceutically acceptable composition. The T cells are preferably contacted with at least one antimitotic agent ex vivo, in vitro, or outside the body of the subject for which the treated T cells and/or the extracellular vesicles released from said T cells are intended as a medicament.

In its use as a medicament, the treated T cells and/or the extracellular vesicles released from said T cells are administered to a subject in an effective amount. It will be understood by the skilled person that the effective amount of the treated T cells and/or the extracellular vesicles released from said T cells may depend on various factors including but not limited to the weight of the patients, the age of the patient, the severity of the condition to be treated, the type of condition to be treated, for example, the type of cancer to be treated, the way of administration of the treated T cells, the type of T cells used, and the dosing regimen that is contemplated. An effective dose of treated T cells can typically be established using dose escalation and is contemplated to be in the range of 10\hat{ }3 to 10\hat{ }9 treated T-cells/kg of the subject. An effective amount of extracellular vesicles released from said T cells is contemplated to be related to an effective amount of treated T cells. For example, the number of extracellular vesicles released from said T cells to be used as an effective amount as a medicament may equal (or may be a percentage thereof, for example 10-200%) to the number of extracellular vesicles that would be released from an effective amount of treated T cells.

The treated T cells and/or the extracellular vesicles released from said T cells can be provided to a subject as a onetime treatment, or according to a dosing regimen, for example once, once every year, once every month, once every week, once every day, once every other day, once every other two, three, four, five or six day, and combinations thereof.

Antimitotic agents are well known to the skilled person, in particular in the field of treatment of cancer. These agents are among the most common agents or drugs used in the treatment of cancer or are being developed for that purpose. Antimitotic agents are thought to cause cells to arrest in the metaphase for some period of time prior to an aberrant exit from mitosis into a state called ‘mitotic catastrophe’. This is believed to activates a death pathway leading to cancer cell death.

Examples of antimitotic agents include taxanes and vinca alkaloids. Taxanes and vinca alkaloids are also sometimes referred to as anti-tubulin agents.

Taxanes, and pharmaceutically acceptable salts thereof, are a class of terpene-based microtubule disruptors derived from or structurally related to taxol. Microtubules are essential to cell division, and taxanes are believed to stabilize GDP-bound tubulin in the microtubule, thereby inhibiting the process of cell division as depolymerization is prevented, making taxanes mitotic inhibitors. Taxanes that can be used in the context of the current invention include but are not limited to paclitaxel, docetaxel, cabazitaxel, taxol, hongdoushan A, hongdoushan B, hongdoushan C, baccatin I, or baccatin II. Taxanes can further include, but are not limited to 10-deacetylbaccatins, or derivatives thereof, such as 10-Deacetylbaccatin III, 10-Deacetylbaccatin V, 10-Deacetylbaccatin VI, 13-Epi-10-Deacetylbaccatin III, 13-[3-(2-Naphthyl) prop-2-enoyl]-2-debenzoyl-2-(4-methoxybenzoxyl)-10-deacetylbaccatin III, or 10-N,N-Dimethylglycyl-13-[3-(2-Naphthyl) prop-2-enoyl]-10-deacetylbaccatin III.

Also contemplated are conjugates or complexes comprising taxanes such as nanoparticle albumin-bound (“nab-”) taxanes such as nab-paclitaxel and nab-docetaxel. Also contemplated are derivates of taxanes having similar biological activity as disclosed herein.

Vinca-alkaloids, and pharmaceutically acceptable salts thereof, have been obtained from the Madagascar periwinkle plant, and have been used to treat diabetes, high blood pressure, and various cancers. It is believed that vinca alkaloids prevent mitotic spindle formation through inhibition of tubulin polymerization. Examples of vinca-alkaloids that can be used in the context of the current invention include but are not limited to vincristine, vinblastine, vindesine, vinorelbine, desoxyvincaminol, vincaminol, vinburnine, vincamajine, and vineridine. Typically, there are four major vinca alkaloids in clinical use: vinblastine, vinorelbine, vincristine, and vindesine.

Also contemplated are conjugates or complexes comprising vinca alkaloids, such as nanoparticle-bound vinca alkaloids. Also contemplated are derivates of vinca alkaloids having similar biological activity as disclosed herein.

Although the classic approach is that vinca alkaloids depolymerize microtubules, thereby increasing the soluble tubulin pool, whereas taxanes stabilize microtubules and increase the microtubular mass, data also suggest that both classes of agents have a similar mechanism of action, involving the inhibition of microtubule dynamics (Dumontet, Journal of Clinical Oncology 17, no. 3 (Mar. 1, 1999) 1061-1061).

There is also provided for T cells and/or extracellular vesicles for use as a medicament according to the invention, wherein the T cells and/or extracellular vesicles are obtained by:
(i) providing isolated T cells;
(ii) contacting the provided T cells with at least one antimitotic agent, preferably selected from the groups consisting of a taxane and a vinca alkaloid, preferably in an aqueous medium; and
(iii) obtaining the T cells contacted with the at least one antimitotic agent, and/or obtaining the extracellular vesicles released from said T cells.

The skilled person understands how to provide for isolated T cells, how to contact these T cells with at least one antimitotic agent, and how to obtain the T cells contacted with the at least one antimitotic agent, and/or how to obtain the extracellular vesicles released from said T cells. The skilled person may, without being limited thereto, for example use methods as disclosed herein in the Examples, or methods similar thereto.

Isolated T cell are provided. The term “isolated T cells” as used herein refers to T cells being present in a non-naturally occurring environment, e. g. separated from their naturally occurring environment. For example, an isolated T cell relates to a T cell that is no longer in its natural environment, for example, in vitro, ex vivo or in a host. The term, next to being isolated from naturally occurring source, also refers to such T cell being artificially produced. As an example, T cells that have been obtained from an animal (and are no longer present in said animal) are considered to be “isolated T cells” within the context of the current invention. Also contemplated as being “isolated T cells” within the context of the current invention, in particular in context to the use of the treated T cells as a medicament, are T cells that are not present in the subject for which the treated T cells are intended. For example, the isolated T cells may be ex vivo, in vitro, but may, is some embodiments also be T cells that are present in another animal or subject than the subject that is intended to be treated with the treated T cells. In such embodiment, for example, T cells in a first animal are contacted with at least one antimitotic agent, preferably selected from the groups consisting of a taxane and a vinca alkaloid, the thus treated T cells are collected from said first animal and subsequently transferred to a second animal (e.g., the subject intended to be treated with the treated T cells). Yet, in the practice of the current invention it is contemplated, and preferred, that the isolated T cells are present ex vivo, in vitro, or outside an (animal) body.

Methods for obtaining T cells are well known to the skilled person and include obtaining cells from blood, spleen, and lymph node. T cells may be obtained from a healthy subject or from a diseased subject. T cells may also be obtained from a tumor. A skilled person understands that within the context of the current invention, it is not required that the isolated T cells are pure or devoid of any other cell type. For example, other types of cells, such as B cells, NK cells, or other mononuclear cells may be present without having any undesired effect in the context of the current invention. Therefore, in some embodiments, the T cells make up 1-100% (number of cells) of a cell population comprising the isolated T cells. Preferably the cell population to be contacted (treated) with the at least one antimitotic agent, preferably selected from the groups consisting of a taxane and a vinca alkaloid comprises at least 1, 5, 10, 20, 25, 30, 40, 50, 75, 90, 95, 98, 99, or 100% T cells.

In some embodiments, the T cells are T cells that have been obtained in vitro from stem cells, induced pluripotent stem cells, hemogenic endothelial cells, or hematopoietic stem cells. In some embodiment, the providing of isolated T cells may thus include the obtaining of these T cells by proliferation and/or differentiating stem cells, induced pluripotent stem cells, hemogenic endothelial cells, or hematopoietic stem cells. The skilled person knows how to proliferation and/or differentiating stem cells, induced pluripotent stem cells, hemogenic endothelial cells, or hematopoietic stem cells in order to obtain T cells that are suitable for use in the current invention.

The isolated T cells may be provided in any suitable medium, preferably aqueous medium. For example, the T cells may be provided in serum, in blood or in an artificial medium, included buffered aqueous media such as PBS. Preferably, the T cells are provided in a medium that is serum-free.

In a next step, the isolated T cells are contacted with at least one antimitotic agent, preferably selected from the groups consisting of a taxane and a vinca alkaloid.

The skilled person, based on the disclosure herein, understand how to contact the isolated T cells with the at least one antimitotic agent, preferably selected from the groups consisting of a taxane and a vinca alkaloid.

For example, the skilled person understands that the concentration of the antimitotic agent to contact the isolated T cells with may depend on the presence of other cells or other compounds in the medium, the type of T cells, the number or concentration of T cells and the like. The skilled person also understands that, typically, the concentration of the antimitotic agent should not be so high that it kills the isolated T cells and/or that it is substantially toxic for the T cells. For example, in the Examples a concentration of 5-10 nmol/L docetaxel was used, and the skilled person is able to establish a concentration of the antimitotic agent, preferably selected from the groups consisting of a taxane and a vinca alkaloid, which can be suitable used to contact the isolated T cells within the context of the current invention. In some embodiments, the concentration of the taxane is, for example at least 0.01 nmol/L, 0.05 nmol/L. 0.1 nmol/L, 0.5 nmol/l, 0.8 nmol/L, 1.0 nmol/L, or. 2.0 nmol/L. In some embodiments the concentration of taxane is no more than 500 nmol/L, 100 nmol/L, 50 nmol/L or 25 nmol/L. In some embodiments, the concentration of the taxane is least 0.01 nmol/L, preferably between 0.1 nmol/L and 500 nmol/L, preferably between 2 and 50 nmol/L Other preferred concentrations are disclosed herein elsewhere.

In some embodiments, the concentration of the vinca alkaloid is, for example, at least 0.01 nmol/L, preferably between 0.1 nmol/L and 500 nmol/L, preferably between 2 and 50 nmol/L, more preferably between 2 and 15 nmol/L. In some embodiments, the concentration of the vinca alkaloid is, for example at least 0.01 nmol/L, 0.05 nmol/L. 0.1 nmol/L, 0.5 nmol/l, 0.8 nmol/L, 1.0 nmol/L, or. 2.0 nmol/L. In some embodiments the concentration of vinca alkaloid is no more than 500 nmol/L, 100 nmol/L, 50 nmol/mol or 25 nmol/L. Other preferred concentrations are disclosed herein elsewhere.

The skilled person also knows how to select a suitable period of contacting the isolated T cells with the antimitotic agent, preferably selected from the groups consisting of a taxane and a vinca alkaloid. For example, in the Examples, contacting of the isolated T cells was performed for a period of 72 hours. In some embodiment it is contemplated that contacting of the T cells with the antimitotic agent, preferably selected from the groups consisting of a taxane and a vinca alkaloid, is for a period of at least 2 hours, at least 4 hours, at least 8, 10, 12, 24, 36, 48, 60, or at least 72 hours.

The skilled person also knows how to select a suitable temperature for the incubation of the isolated T cells with the antimitotic agent, preferably selected from the groups consisting of a taxane and a vinca alkaloid. Preferably the temperature is at least 4 degrees Celsius, preferably at least 14 degrees Celsius, preferably between 30 degrees Celsius and 39 degrees Celsius, most preferably no more than 38 degrees Celsius.

More than one antimitotic agent may be used, for example a combination of one, two or three different antimitotic agents, including those disclosed herein, may be used. For example, a combination of at least one taxane and at least one vinca alkaloid may be used. For example, a combination of more than one vinca alkaloid may be used. For example, a combination of more than one taxane may be used. Preferably the vinca alkaloid is a vinca alkaloid as disclosed herein. Preferably the taxane is a taxane as disclosed herein. Preferably the antimitotic agent is a taxane. Preferably no more than one antimitotic agent is used.

During or after the isolated T cells are brought into contact with the antimitotic agent, preferably selected from the groups consisting of a taxane and a vinca alkaloid, the contacted (treated) T cells and/or the extracellular vesicles released from said T cells may be obtained. Again, the skilled person understands how to obtain the treated T cells and/or the extracellular vesicles released from said T cells, for example using methods well-known from the prior art, including these described herein in the Examples, and similar methods.

It will be understood that the step of obtaining the contacted (treated) T cells and/or the extracellular vesicles released from said T cells make consist of obtaining part or all of the medium in which the contacting of the isolated T cells with the antimitotic agent was performed. However, in a preferred embodiment the step of obtaining the contacted (treated) T cells and/or the extracellular vesicles released from said T cells comprises separating, at least to a certain extent, the T cells and/or the extracellular vesicles released from said T cells from the medium (containing the antimitotic agent) in which the contacting of the isolated T cells with the antimitotic agent was performed. In order words, in a preferred embodiment the contacted (treated) T cells and/or the extracellular vesicles released from said T cells are separated, at least to a certain extent, from the antimitotic agent that contacted the isolated T cells. This can be performed using method well-known to the skilled person, including these described herein, or similar methods. For example, the T cells and/or the extracellular vesicles released from said T cells may be obtained by methods involving, but not limited to, for example, washing, centrifugation, dialysis and/or the use of size-exclusion columns. In a preferred embodiment, the contacted (treated) T cells are obtained. In a preferred embodiment, the extracellular vesicles released from said T cells are obtained. In a preferred embodiment both the contacted (treated) T cells and the extracellular vesicles released from said T cells are obtained. Depending on the method for obtaining the contacted (treated) T cells and/or the extracellular vesicles released from said T cells, other cells, originally present in the cell population comprising the isolated T cells may still be present in the obtained contacted (treated) T cells and/or the extracellular vesicles released from said T cells. The thus obtained contacted (treated) T cells and the extracellular vesicles released from said T cells may direct be used as a medicament or may be further treated, for example by removing undesired other cells or compounds, by adding preservatives and/or other medicaments to the obtained treated) T cells and/or the extracellular vesicles released from said T cells.

There is also provided for T cells and/or extracellular vesicles for use as a medicament according to the invention, wherein the use as a medicament comprises administering the T cells and/or the extracellular vesicles to a subject.

The T cells and/or extracellular vesicles for use as a medicament according to the invention, i.e., the “treated T cells” and/or extracellular vesicles released from said T cells may be administered to a subject in need thereof, as is detailed herein elsewhere, in the treatment of various conditions. In one embodiment the treated T cells are administered to the subject. In one embodiment the extracellular vesicles released from said T cells are administered to the subject. In one embodiment both the treated T cells and the extracellular vesicles released from said T cells are administered to a subject in need thereof.

In some embodiments, the treated T cells and/or extracellular vesicles released from said T cells are administered to the subject directly after the treated T cells and/or extracellular vesicles released from said T cells have been obtained. In some embodiments, the treated T cells and/or extracellular vesicles released from said T cells are stored after they have been obtained. For example, the treated T cells and/or extracellular vesicles released from said T cells may be stored at reduced temperature, for example below 10 degrees Celsius, in the form of a pharmaceutical acceptable composition, and subsequent be used as a medicament and administered to a subject in need thereof. In some embodiments the treated T cells and/or extracellular vesicles released from said T cells may be stored at a temperature below 0 degrees Celsius, for example in a suitable medium that prevent freezing damage, for example, comprising glycerol, DMSO (e.g., in a 10% concentration). Therefore, in this embodiment there is provided for a composition, preferably an aqueous composition, preferably an aqueous solution suitable for injection and/or infusion, comprising treated T cells and/or extracellular vesicles released from said T cells according to the invention and at least one or two compounds selected from glycerol, ethylene glycol, and DMSO (dimethyl sulphoxide), for example in a percentage of more than or equal to 2, 5, 7, 9, 10, 12, 15 or 20 (wt./vol.). Additionally, the composition may further comprise one or more compounds selected from the group consisting of sugars, such as dextrose, proteins, such as albumin, salts, polysaccharides, vitamins, minerals, and water. A skilled person is well aware of further excipients used for obtaining a pharmaceutically acceptable composition as meant and disclosed herein.

Once the treated T cells and/or extracellular vesicles released from said T cells are to be administered to a subject, this may be done by any suitable method, including by injection and/or infusion, e.g. by intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, including intratumoral inject and/or infusion. The skilled person is well aware of suitable means and ways of administering the treated T cells and/or extracellular vesicles released from said T cells to a subject, in accordance with the disclosure herein. For example, the treated T cells and/or extracellular vesicles released from said T cells may be administered in accordance with a dosage regimen such as those described herein elsewhere and may be part of a combination treatment with other drugs or medicaments to the subject in need thereof. The treated T cells and/or extracellular vesicles released from said T cells may be administered simultaneously or separately from such other drugs or medicaments, for example such drugs or medicaments as described herein elsewhere, including anthracyclines, taxanes and/or vinca alkaloids. Thus, in some embodiments there is provided for use of the treated T cells and/or extracellular vesicles released from said T cells as a medicament in combination with the use of other medicaments, including, for example, anthracyclines, taxanes and/or vinca alkaloids. It is contemplated that the treated T cells and/or extracellular vesicles released from said T cells as a medicament may allow for reduced dosages or frequencies of use of other drugs, such as anthracyclines, and in particular taxanes and/or vinca alkaloids, thereby reducing possible adverse effects that are associated with the use of these other medicament, such as anthracyclines, and in particular taxanes and/or vinca alkaloids.

Preferably the subject is a human subject.

There is also provided for T cells (i.e., “treated T cells”) and/or extracellular vesicles for use as a medicament according to the invention, wherein the use as a medicament comprises:
(i) providing isolated T cells;
(ii) contacting the provided T cells with at least one antimitotic agent, preferably selected from the groups consisting of a taxane and a vinca alkaloid;
(iii) obtaining the T cells (i.e., “treated T cells”) contacted with the at least one antimitotic agent, and/or obtaining the extracellular vesicles released from said T cells; and
(iv) administering the obtained T cells (i.e., “treated T cells”) and/or the obtained extracellular vesicles to a subject.

Details and preferences with respect to the various aspects of this embodiment are already described herein elsewhere and likewise apply to this embodiment and will, for conciseness, not be repeated here.

There is also provided for T cells (i.e., “treated T cells”) and/or extracellular vesicles for use as a medicament according to the invention, wherein the T cells comprise CD4+ T cells and/or CD8+ T cells and/or wherein the T cells are autologous T cells, allogenic T cells or syngeneic T cells.

There is also provided for T cells and/or extracellular vesicles released from said T cells for use as a medicament according to the invention, wherein the T cells comprise cytotoxic T-lymphocytes, regulatory T-lymphocytes, or helper T-lymphocytes. In some embodiments, the T cells, treated or to be treated with the antimitotic agent, preferably selected from the groups consisting of a taxane and a vinca alkaloid, is a tumor reactive T cell and/or a tumor specific T cell.

It was surprisingly found that the killing of cancer cells induced by the T cells obtainable/obtained by contacting (isolated) T cells with at least one antimitotic agent, preferably selected from the groups consisting of a taxane and a vinca alkaloid, and/or extracellular vesicles released from said T cells, is independent on the nature of the T cell, as is shown in the Examples. The T cells within the context of the invention may, for example, be naïve T cells, non-naïve T cells, tumor-reactive T cells. The T cells may be immature T cells or mature T cells. The T cells may also be CD4+ T cells and/or CD8+ T cells.

CD4+ cells or CD4-positive cells, are a well-known type of T cell that play an important role in the adaptive immune system. They aid the activity of other immune cells by releasing cytokines and are considered essential in B cell antibody class switching, breaking cross-tolerance in dendritic cells, in the activation and growth of cytotoxic T cells, and in maximizing bactericidal activity of phagocytes such as macrophages and neutrophils. CD4+ cells are mature Th (T helper) cells that express the surface protein CD4.

CD8+ T cells are also known as cytotoxic T cells, CTL, T-killer cell, cytolytic T cell, or killer T cell. CD8+ T cells are involved in fighting cancer cells, cells that are infected by intracellular pathogens (such as viruses or bacteria), or cells that are damaged. Most CD8+ T cells express T-cell receptors (TCRs) that can recognize a specific antigen. The TCR is accompanied by a glycoprotein called CD8.

Within the context of the current invention, and in some embodiments, the T cells are CAR T cells and/or genetically modified T cells.

In some embodiments, the T cells are autologous T cells, allogenic T cells or syngeneic T cells. The T cells may be tumor infiltrating lymphocytes (TIL), for example, as part of TIL therapy, CAR T cells, or other lymphocyte-based treatment. As is well known to the skilled person, autologous T cells are T cells that have been obtained from or are derived from T cells from the subject that is to be treated with the “treated” T cells and/or extracellular vesicles released from said T cells (“treated T cells). For example, the autologous T cells according to the invention, may be used as a medicament, for example in TIL therapy or any other lymphocytes-based therapy, such as CAR T cell therapy. Allogenic T cells have been obtained from or are derived from T cells from a (healthy) donor, e.g., someone else than the subject to be treated. Syngeneic T cells refers to T cells that are genetically similar or identical and hence immunologically compatible with the subject to be treated. As described herein elsewhere, the T cells may also be obtained by in vitro proliferation and/or differentiation of stem cells, induced pluripotent stem cells, hemogenic endothelial cells, or hematopoietic stem cells. It is therefore provided that the T cells in accordance with the current invention may be used in adoptive cell therapy, including therapy using tumor infiltrating lymphocytes/tumor associated lymphocytes (TILs), and wherein, for example, the TIL are the T cells contacted with at least one antimitotic agent. As the skilled person knows, adoptive cell therapy (ACT) using autologous T-cells to mediate cancer regression has shown much promise in early clinical trials. Several general approaches have been taken such as the use of naturally occurring tumor reactive or tumor infiltrating lymphocytes/Tumor associated lymphocytes (TILs) expanded ex vivo. Additionally, T-cells may be modified genetically to retarget them towards defined tumor antigens. This can be achieved via the gene transfer of: peptide (p)-major histocompatibility complex (MHC) specific T-cell Receptors (TCRs); or synthetic fusions between tumor specific single chain antibody fragment (scFv) and T-cell signaling domains (e.g., Oü3z), the latter being termed chimeric antigen receptors (CARs). TIL and TCR transfer has proven particularly effective when targeting melanoma (Rosenberg et al., 2011; Morgan et al., 2006), whereas CAR therapy has shown much promise in the treatment of certain B-cell malignancies (Grupp et al., 2013). As will be understood by the skilled person, T cells used in these type of therapies may be the T cells according to the invention, and, for example, be the isolated T cells that are contacted with at least one antimitotic agent. In another embodiment, the T cells according to the invention, and that have been contacted with the at least one antimitotic agent, is added to the therapy, in addition to the standard therapy. Furthermore, in some embodiments, the (autologous) T cells according to the invention, may be used as a medicament, for example in TIL therapy or any other lymphocytes-based therapy, such as CAR T cell therapy, and in any indication where those therapies can be employed. In some embodiments, the (autologous) T cells according to the invention can be used as medicament in TIL therapy or any other lymphocytes-based therapy, such as CAR T cell therapy, and wherein the subject to be treated is in need of such TIL therapy or any other lymphocytes-based therapy, such as CAR T cell therapy. The skilled person understands that the indication to be treated may include cancer, for example skin cancer, melanoma, or lung cancer, but is not limited thereto, and includes any indication that may benefit from the treatment with T cells, for example autologous T cells. In an embodiment it is, for example, contemplated the T cells (and/or extracellular vesicles released from said T cells (after said T cells are contacted with the at least one antimitotic agent as described herein) according to the invention are used in the treatment of patients, preferably non-responders, i.e. subjects that do not or only to a limited extent respond to or benefit from a (standard) treatment, for example to cancer treatment, for example to cell therapy, for example to T cell therapy, for example to CAR T cell therapy or TIL therapy. It is contemplated that the (autologous) T cells according to the invention can be employed in addition to or instead of such (standard) treatment, preferably, and to which (standard) treatment the subject shows only limited response or no response at all. Therefore, in one embodiment there is provided for the T cells (and/or extracellular vesicles released from said T cells) according to the invention for use as a medicament, and wherein, for example, the treatment further comprises treatment with a further medicament, preferably a further medicament for the same indication. In another embodiment there is provided for the use of the T cells (and/or extracellular vesicles) according to the invention in the treatment of subject that did not respond to previous treatment,—for example that are non-responders to (standard) T cell therapy, TIL therapy or CART therapy. There is also provided for T cells (i.e., “treated T cells”) and/or extracellular vesicles released from said “treated” T cells for use as a medicament in therapy wherein the therapy is TIL therapy. There is also provided for T cells (i.e., “treated T cells”) and/or extracellular vesicles released from said “treated” T cells for use as a medicament in therapy wherein the therapy is TIL therapy and wherein the T cells (i.e., “treated T cells”) and/or extracellular vesicles release from said “treated” T cells are administered to a subject, preferably a subject in need of such treatment. There is also provided for T cells (i.e., “treated T cells”) and/or extracellular vesicles released from said “treated” T cells for use as a medicament in therapy wherein the therapy is TIL therapy and wherein the T cells (i.e., “treated T cells”) and/or extracellular vesicles released from said “treated” T cells are administered to a subject in an effective dose. There is also provided for T cells (i.e., “treated T cells”) and/or extracellular vesicles released from said “treated” T cells for use as a medicament in therapy wherein the therapy is TIL therapy and wherein the T cells (i.e., “treated T cells”) and/or extracellular vesicles released from said “treated” T cells are administered on one or more than one occasion, for example repeatedly.

Tumor Infiltrating Lymphocyte therapy has been applied to a number of different malignancies including gastric (Xu et al., 1995), renal (Figlin et al., 1997; Goedegebuure et al., 1995), cervical (Stevanovic et al., 2015) and colorectal cancers (Gardini et al., 2004), but has been most widely applied and shown most development and promise in melanoma therapy. Trials in advanced metastatic melanoma have consistently shown around 50% response rate with 15-20% complete response (cure) (Rosenberg et al., 2011; Dudley et al., 2010). Additionally, Tumor associated lymphocytes can be obtained from ascites and grown in the same manner as TIL and are also contemplated for use in the current invention.

There is also provide for T cells (i.e. “treated T cells”) and/or extracellular vesicles released from said “treated” T cells for use as a medicament according to the invention, wherein the at least one antimitotic agent is a vinca alkaloid, preferably wherein the vinca alkaloid is selected from the group consisting of vinblastine, vinorelbine, vincristine, vindesine and vinflunine, and/or, wherein the at least one antimitotic agent is a taxane, preferably selected from the groups consisting of paclitaxel, docetaxel, and combinations thereof.

Suitable vinca alkaloids for use in the invention include, but are not limited to vincristine, vinblastine, vindesine, vinorelbine, desoxyvincaminol, vincaminol, vinburnine, vincamajine, and vineridine. Preferably the vinca alkaloid is selected from the group consisting of vinblastine, vinorelbine, vincristine, and vindesine, or any combination thereof.

These vinca alkaloids may be used contacting the T cells in order to obtain the treated T cells according to the invention. Independently, within the context of the current invention, these vinca alkaloids may also be used as a further medicament in combination with the treated T cells and/or extracellular vesicles release from said treated T cells as a medicament in the treatment of a subject.

Suitable taxanes for use in the invention include, but are not limited to paclitaxel, docetaxel, cabazitaxel, taxol, hongdoushan A, hongdoushan B, hongdoushan C, baccatin I, or baccatin II, 10-deacetylbaccatins, or derivatives thereof, such as 10-Deacetylbaccatin III, 10-Deacetylbaccatin V, 10-Deacetylbaccatin VI, 13-Epi-10-Deacetylbaccatin III, 13-[3-(2-Naphthyl) prop-2-enoyl]-2-debenzoyl-2-(4-methoxybenzoxyl)-10-deacetylbaccatin III, or 10-N,N-Dimethylglycyl-13-[3-(2-Naphthyl) prop-2-enoyl]-10-deacetylbaccatin III. Preferably the taxane is selected from the group consisting of paclitaxel, docetaxel, cabazitaxel, taxol, hongdoushan A, hongdoushan B, hongdoushan C, baccatin I, and baccatin II, more preferably paclitaxel, docetaxel, cabazitaxel, or taxol, even more preferably paclitaxel or docetaxel, or combinations thereof.

Also contemplated are conjugates or complexes comprising taxanes such as nanoparticle albumin-bound (“nab-”) taxanes such as nab-paclitaxel and nab-docetaxel.

These taxanes may be used contacting the T cells in order to obtain the treated T cells according to the invention. Independently, within the context of the current invention, these taxanes may also be used as a further medicament in combination with the treated T cells and/or extracellular vesicles release from said treated T cells as a medicament in the treatment of a subject.

There is also provided for T cells ((i.e., “treated T cells”) and/or extracellular vesicles secreted by such T cells for use as a medicament in the treatment of cancer in a subject, and/or for use as a medicament in the prevention of neuropathy, preferably peripheral neuropathy, vinca alkaloid-induced neuropathy and/or taxane-induced neuropathy in a subject.

As explained herein elsewhere, it was surprisingly found by the inventors that T cells treated with a least one antimitotic agent, preferably selected from the groups consisting of a taxane and a vinca alkaloid, and/or extracellular vesicles released from said T cells ((i.e., “treated T cells”) selectively kill cancer cells when subsequently administered to a subject. The invention thus allows for a new and effective therapeutic approach, based on T cells pre-treated with an antimitotic agent, in particular a taxane or a vinca alkaloid, and in particular in the treatment of cancer in a subject.

From the Examples it follows that the treatment of cancer is not limited to a particular type of cancer, and it is contemplated that any type of cancer may be treated in accordance with the invention.

In some embodiments, the cancer is a primary cancer. In some embodiments the cancer is a secondary cancer. In some embodiments the cancer is a cancer cell line. In some embodiments the cancer is a solid tumor. In some embodiments, the solid tumor is a solid cold tumor in which neoantigens are rare, e.g., tumors found in patients defined by a low number of immunogenic antigens (Blank et al. (2016), Science, Vol 352 Issue 6286, pp 658-660). In some embodiments the cancer is a non-solid tumor or a blood tumor. Treatment of a cancer in a subject includes the treatment of a tumor in the subject.

In some embodiments, the cancer in the subject is selected from the group consisting of breast cancer, lung cancer, ovarian cancer, bladder cancer, prostate cancer, a sarcoma, a carcinoma, esophageal cancer, melanoma, glioma, glioblastoma, liver cancer, colon cancer, rectal cancer, leukemia, lymphoma, Hodgkin's disease, multiple myelomas, and cholangiocarcinoma.

As explained herein elsewhere, it was surprisingly found by the inventors that T cells treated with a least one antimitotic agent, preferably selected from the groups consisting of a taxane and a vinca alkaloid, and/or extracellular vesicles released from said T cells ((i.e., “treated T cells”) selectively kill cancer cells when subsequently administered to a subject. The invention thus allows for a new and effective therapeutic approach, based on T cells pre-treated with an antimitotic agent, in particular a taxane or a vinca alkaloid, and in particular in the prevention on neuropathy.

The new therapy as disclosed herein can prevent neuropathy, preferably peripheral neuropathy, for example vinca alkaloid-induced neuropathy and/or taxane-induced neuropathy in a subject. Such peripheral neuropathies induced by chemotherapy (CIPN) are recognized as being an increasingly frequent problem.

Chemotherapy induced peripheral neuropathy (CIPN), including taxane induced peripheral neuropathy and/or vinca alkaloid induced peripheral neuropathy is a common and challenging complication arising from treatment with many commonly used anti-cancer agents. A number of factors have contributed to the increasing prevalence of CIPN, including an increased incidence of cancer, with improved survival and cancer cure rates.

CIPN can occur acutely, during chemotherapy. If severe, it can require a reduction in the dose of chemotherapy, or even stopping prior to completing the planned course. This may have implications for efficacy of oncological treatment and survival. Acute CIPN may resolve after finishing chemotherapy, however, in a number of cases, it will persist, resulting in chronic symptoms, months, or even years later. In some cases, CIPN can emerge shortly after finishing chemotherapy, a phenomenon known as “coasting”. For neurotoxic chemotherapy overall, the prevalence of CIPN 1 month after finishing chemotherapy is around 68%, with this dropping to 60% at 3 months and 30% at 6 months or more (Colvin, Pain. 2019 May; 160 (Suppl 1): S1-S10.). Though the incidence, characteristics, and pathogenesis of chemotherapy induced peripheral neuropathy (CIPN) have been extensively studied, diagnostic guidelines extent only recently (van Haren, Cancer Reports. 2021; e1577).

Amongst the most frequent substances causing peripheral neuropathies are, next to platin compounds, antimitotic agents such as vinca alkaloids and taxanes. Such neuropathy is a common adverse effect of systemic treatment with these antimitotic agents. Surprisingly the invention allows to reduce systemic exposure to such antimitotic agents while at the same time providing therapeutic effect of such antimitotic agents to the subject by treating T cells with the antimitotic agents before providing these to the subject.

The pre-treated T cells are therefor in particular useful as a medicament in the treatment of various conditions that would normally involve treatment with an antimitotic agent, such as a taxane or a vinca alkaloid. In particular the pre-treated T cells can be used in the treatment of cancer and/or in the prevention of neuropathy, for example vinca alkaloid-induced neuropathy and/or taxane-induced neuropathy (i.e., can be used in a treatment of a condition in a subject that would benefit from the use of an antimitotic agent, such as a taxane or a vinca alkaloid, without inducing adverse effects, such as neuropathy, associated with the systemic use of such antimitotic agent).

The treatment of the T cells outside the subject and the subsequent transfer of the T cells and/or extracellular vesicles released from said treated T cells to the subject importantly avoids toxicity that is normally associated with systemic treatment with antimitotic agents.

In some embodiments the T cells (i.e., “treated T cells”) and/or extracellular vesicles secreted by such T cells for use as a medicament in the treatment of cancer is combined with the use of further medicaments or other anti-cancer treatments.

In some embodiments the T cells (i.e., “treated T cells”) and/or extracellular vesicles secreted by such T cells for use as a medicament in the prevention of neuropathy, preferably peripheral neuropathy, vinca alkaloid-induced neuropathy and/or taxane-induced neuropathy in a subject, for example as described herein elsewhere is combined with the use of further medicaments or further anti-cancer treatments.

Non-limiting examples of further medicaments or further anti-cancer treatments that can be combined with the T cells (i.e., “treated T cells”) and/or extracellular vesicles secreted by such T cells as a medicament according to the invention include surgery, such as open surgery, laparoscopic surgery, minimal resection surgery, debulking surgery, complete resection surgery, and so on. Other examples include radio-surgery, RF ablation, and focused ultrasound. Other examples of further therapy include ionizing radiation using various dosing and focusing regimen, for example whole organ radiation, regional radiation, focal radiation, single dose radiation, fractionated dose radiation, and hyper-fractionated dose radiation.

The further therapeutic agent or medicament may be administered simultaneously with, optionally as a component of the same pharmaceutical preparation, or before or after administration of the T cells (i.e., “treated T cells”) and/or extracellular vesicles secreted by such T cells according to the invention.

In certain embodiments, the further medicament or additional therapeutic agent may be, without limitation, a member selected from the group consisting of an anti-cancer therapeutic agent, an inhibitor of an immunomodulatory receptor, an anti-emetic, an MTOR (mammalian target of rapamycin) inhibitor, a cytotoxic agent, a platinum agent, an EGFR inhibitor, a VEGF inhibitor, a microtubule stabilizer, a taxane, a CD20 inhibitor, a CD52 inhibitor, a CD30 inhibitor, a RANK (Receptor activator of nuclear factor kappa-B) inhibitor, a RANKL (Receptor activator of nuclear factor kappa-B ligand) inhibitor, an ERK inhibitor, a MAP Kinase inhibitor, an AKT inhibitor, a MEK inhibitor, a PI3K inhibitor, a HER1 inhibitor, a HER2 inhibitor, a HER3 inhibitor, a HER4 inhibitor, a Bcl2 inhibitor, a CD22 inhibitor, a CD79b inhibitor, an ErbB2 inhibitor, a farnesyl protein transferase inhibitor, an anti-PD1 antibody or an antigen-binding fragment thereof, an anti-PDL1 antibody or an antigen-binding fragment thereof, pembrolizumab, nivolumab, CT-011, anti-CTLA4 antibody or an antigen-binding fragment thereof, anti-TIM3 antibody or an antigen-binding fragment thereof, anti-CS 1 antibody or an antigen-binding fragment thereof, elotuzumab, anti-KIR2DL1/2/3 antibody or an antigen-binding fragment thereof, lirilumab, anti-CD137 antibody or an antigen-binding fragment thereof, urelumab, anti-GITR antibody or an antigen-binding fragment thereof, TRX518, anti-PD-L1 antibody or an antigen-binding fragment thereof, BMS-936559, MSB0010718C, MPDL3280A, anti-PD-L2 antibody or an antigen-binding fragment thereof, anti-ILT1 antibody or an antigen-binding fragment thereof, anti-CEACAM1 antibody or antigen-binding fragment thereof, anti-ILT2 antibody or an antigen-binding fragment thereof, anti-ILT3 antibody or an antigen-binding fragment thereof, anti-ILT4 antibody or an antigen-binding fragment thereof, anti-ILT5 antibody or an antigen-binding fragment thereof, anti-ILT6 antibody or an antigen-binding fragment thereof, anti-ILT7 antibody or an antigen-binding fragment thereof, anti-ILT8 antibody or an antigen-binding fragment thereof, anti-CD40 antibody or an antigen-binding fragment thereof, anti-OX40 antibody or an antigen-binding fragment thereof, anti-CD137 antibody or an antigen-binding fragment thereof, anti-KIR2DL1 antibody or an antigen-binding fragment thereof, anti-KIR2DL2/3 antibody or an antigen-binding fragment thereof, anti-KIR2DL4 antibody or an antigen-binding fragment thereof, anti-KIR2DL5A antibody or an antigen-binding fragment thereof, anti-KIR2DL5B antibody or an antigen-binding fragment thereof, anti-KIR3DL1 antibody or an antigen-binding fragment thereof, anti-KIR3DL2 antibody or an antigen-binding fragment thereof, anti-KIR3DL3 antibody or an antigen-binding fragment thereof, anti-NKG2A antibody or an antigen-binding fragment thereof, anti-NKG2C antibody or an antigen-binding fragment thereof, anti-NKG2E antibody or an antigen-binding fragment thereof, IL-10, anti-IL10, anti-TSLP, PEGylated IL-10, 13-cis-retinoic acid, 3-[5-(methylsulfonylpiperadinemethyl)-indolyl]-quinolone, 4-hydroxytamoxifen, 5-deooxyuridine, 5′-deoxy-5-fluorouridine, 5-fluorouracil, 6-mecaptopurine, 7-hydroxystaurosporine, A-443654, abirateroneacetate, abraxane, ABT-578, acolbifene, ADS-100380, aflibercept, ALT-110, altretamine, amifostine, aminoglutethimide, amrubicin, amsacrine, anagrelide, anastrozole, angiostatin, AP-23573, ARQ-197, arzoxifene, AS-252424, AS-605240, asparaginase, AT-9263, ATI3387, atrasentan, axitinib, AZD1152, Bacillus Calmette-Guerin (BCG) vaccine, batabulin, BC-210, BGJ398, besodutox, bevacizumab, bicalutamide, Bio111, BIO1140, BKM120, bleomycin, BMS-214662, BMS-247550, BMS-275291, BMS-310705, bortezimib, buserelin, busulfan, calcitriol, camptothecin, canertinib, capecitabine, carboplatin, carmustine, CC8490, CEA vaccine, cediranib, CG-1521, CG-781, chlamydocin, chlorambucil, chlorotoxin, cilengitide, cimitidine, cisplatin, cladribine, clodronate, cobimetnib, COL-3, CP-724714, cyclophosphamide, cyproterone, cyproteroneacetate, cytarabine, cytosinearabinoside, dabrafenib, dacarbazine, dacinostat, dactinomycin, dalotuzumab, danusertib, dasatanib, daunorubicin, decatanib, deguelin, denileukin, deoxycoformycin, depsipeptide, diarylpropionitrile, diethylstilbestrol, diftitox, DNE03, docetaxel, dovitinib, doxorubicin, droloxifene, edotecarin, yttrium-90 labeled-edotreotide, edotreotide, EKB-569, EMD121974, encorafenib, endostatin, enzalutamide, enzastaurin, epirubicin, epithilone B, ERA-923, erbitux, erlotinib, estradiol, estramustine, etoposide, everolimus, exemestane, ficlatuzumab, finasteride, flavopiridol, floxuridine, fludarabine, fludrocortisone, fluoxymesterone, flutamide, FOLFOX regimen, fulvestrant, galeterone, ganetespib, gefitinib, gemcitabine, gimatecan, glucopyranosyl lipid A, goserelin, goserelin acetate, gossypol, GSK461364, GSK690693, HMR-3339, hydroxyprogesteronecaproate, hydroxyurea, IC87114, idarubicin, idoxyfene, ifosfamide, IM862, imatinib, imiquimod, IMC-1C11, INCB24360, INC280, INO1001, interferon, interleukin-2, interleukin-12, ipilimumab, irinotecan, JNJ-16241199, ketoconazole, KRX-0402, lapatinib, lasofoxifene, LEE011, letrozole, leucovorin, leuprolide, leuprolide acetate, levamisole, liposome entrapped paclitaxel, lomustine, lonafarnib, lucanthone, LY292223, LY292696, LY293646, LY293684, LY294002, LY3009120, LY317615, marimastat, mechlorethamine, medroxyprogesteroneacetate, megestrolacetate, MEK162, melphalan, mercaptopurine, mesna, methotrexate, mithramycin, mitomycin, mitotane, mitoxantrone, tozasertib, MLN8054, a suspension of heat killed Mycobacterium obuense, natitoclax, neovastat, neratinib, neuradiab, nilotinib, nilutimide, nolatrexed, NVP-BEZ235, oblimersen, octreotide, ofatumumab, oregovomab, ornatuzumab, orteronel, oxaliplatin, paclitaxel, palbociclib, pamidronate, panitumumab, pazopanib, PD0325901, PD184352, PEG-interferon, pemetrexed, pentostatin, perifosine, phenylalanine mustard, PI-103, pictilisib, PIK-75, pipendoxifene, PKI-166, plicamycin, PLX8394, poly-ICLC, porfimer, prednisone, procarbazine, progestins, PSK, PX-866, R-763, raloxifene, raltitrexed, razoxin, ridaforolimus, rituximab, romidepsin, RTA744, rubitecan, scriptaid, Sdx102, seliciclib, selumetinib, semaxanib, SF1126, sirolimus, SN36093, sorafenib, spironolactone, squalamine, SR13668, streptozocin, SU6668, suberoylanalide hydroxamic acid, sunitinib, synthetic estrogen, talampanel, talimogene laherparepvec, tamoxifen, temozolomide, temsirolimus, teniposide, tesmilifene, testosterone, tetrandrine, TGX-221, thalidomide, 6-thioguanine, thiotepa, ticilimumab, tipifarnib, tivozanib, TKI-258, TLK286, TNFac, topotecan, toremifene citrate, trabectedin, trametinib, trastuzumab, tretinoin, trichostatin A, triciribinephosphate monohydrate, triptorelin pamoate, TSE-424, uracil mustard, valproic acid, valrubicin, vandetanib, vatalanib, VEGF trap, vemurafenib, vinblastine, vincristine, vindesine, vinorelbine, vitaxin, vitespan, vorinostat, VX-745, wortmannin, Xr311, zanolimumab, ZK186619, ZK-304709, ZM336372, ZSTK474, Z-100, casopitant, netupitant, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, GCSF, PEG-GCSF, erythropoietin, epoetin alfa, darbepoetin alfa, a Bruton's tyrosine kinase (BTK) inhibitor, a prostate specific antigen vaccine, azacitidine, eribulin mesylate, lenvatinib mesylate, epacadostat, an anti-4-1BB agonist antibody or antigen-binding fragment, crizotinib, a CSF1 receptor kinase inhibitor, entinostat, birinapant, and niraparib. Also contemplated are combination with CAR T cells, NK cells and the like.

In some embodiments, the subject is selected from the group consisting of a human, a male, a female, a child, a human that is allergic to an antimitotic agent, preferably selected from the groups consisting of a taxane and a vinca alkaloid, a human that elicits a hypersensitive reaction to an antimitotic agent, preferably selected from the groups consisting of a taxane and a vinca alkaloid taxane, and/or a human that develops a neuropathy during systemic treatment with an antimitotic agent, preferably selected from the groups consisting of a taxane and a vinca alkaloid.

There is also provided for antimitotic agent, preferably selected from the groups consisting of a taxane and a vinca alkaloid, for use in the treatment of cancer in a subject and/or for use in the prevention of neuropathy, preferably peripheral neuropathy, vinca alkaloid-induced neuropathy and/or taxane-induced neuropathy in a subject wherein the use comprises:
(i) providing isolated T cells;
(ii) contacting the provided T cells with the antimitotic agent, preferably selected from the groups consisting of a taxane and a vinca alkaloid, preferably in an aqueous medium;
(iii) obtaining the T cells contacted with the antimitotic agent, and/or obtaining the extracellular vesicles released from said T cells; and
(iv) administering the obtained T cells and/or the obtained extracellular vesicles to a subject.

Details and preferences with respect to the various aspects of this embodiment are already described herein elsewhere and likewise apply to this embodiment and will, for conciseness, not be repeated here.

There is also provided for a method of producing treated T cells, and/or extracellular vesicles released from said T cells, wherein the method comprises the steps of
(a) providing isolated T cells;
(b) contacting the provided T cells with at least one antimitotic agent, preferably selected from the groups consisting of a taxane and a vinca alkaloid, for example in an aqueous medium; and
(c) obtaining the T cells contacted with the at least one antimitotic agent, and/or obtaining the extracellular vesicles released from said T cells, to provide for the treated T cells and/or extracellular vesicles released from said T cells.

There is also provided for a method of producing extracellular vesicles released from treated T cells, wherein the method comprises the steps of
(a) providing isolated T cells;
(b) contacting the provided T cells with at least one antimitotic agent, preferably selected from the groups consisting of a taxane and a vinca alkaloid, for example in an aqueous medium; and
(c) obtaining the T cells contacted with at least one antimitotic agent, and obtaining the extracellular vesicles released from said T cells, to provide for the extracellular vesicles released from said T cells.

There is also provided for a method of producing treated T cells, and/or extracellular vesicles released from said T cells, wherein the method comprises the steps of
(a) providing isolated T cells;
(b) contacting the provided T cells with at least one antimitotic agent consisting of a vinca alkaloid, for example in an aqueous medium; and
(c) obtaining the T cells contacted with at least one antimitotic agent, and/or obtaining the extracellular vesicles released from said T cells, to provide for the treated T cells and/or extracellular vesicles released from said T cells.

In some embodiments the method is for producing a medicament and wherein step (c) comprises obtaining the T cells contacted with the at least one antimitotic agent, and/or obtaining the extracellular vesicles released from said T cells, to provide for the medicament. In some embodiments, the T cells contacted with the at least one antimitotic agent, and/or obtaining the extracellular vesicles released from said T cells are formulated together with a pharmaceutically acceptable carrier, for example a cryoprotectant as disclosed herein elsewhere.

According to the best of the inventor's knowledge, this is the first time a method of producing treated T cells, and/or extracellular vesicles released from said T cells, as previously disclosed in any of the aspects and embodiments, is disclosed for producing a medicament. In particular embodiments, the producing of a medicament is for producing a medicament for the treatment of cancer and/or the treatment is in order to prevent neuropathy, preferably peripheral neuropathy, vinca alkaloid-induced neuropathy and/or taxane-induced neuropathy in a subject.

Details and preferences with respect to the various aspects of this embodiment are already described herein elsewhere and likewise apply to this embodiment and will, for conciseness, not be repeated here.

There is also provided for the method of producing treated T cells, and/or extracellular vesicles released from said T cells according to the invention wherein
in step (b) the provided T cells are contacted with at least one taxane, wherein the taxane concentration is at least 0.01 nmol/L, preferably between 0.1 nmol/L and 500 nmol/L, preferably between 2 and 50 nmol/L;
in step (b) the provided T cells are contacted with at least one vinca alkaloid, wherein the vinca alkaloid concentration is at least 0.01 nmol/L, preferably between 0.1 nmol/L and 500 nmol/L, preferably between 2 and 50 nmol/L, more preferably between 2 and 15 nmol/L;
in step (b) the provided T cells are contacted with the antimitotic agent for a period of at least 1 hour, preferably between 6 hours and 200 hours;
in step (b) the provided T cells are contacted with the antimitotic agent at a temperature of at least 14 degrees Celsius, preferably between 14 degrees Celsius and 45 degrees Celsius;
in step (c) T cells are obtained by isolating the T cells from the medium in which the contacting of step (b) is performed; and/or
in step (c) the extracellular vesicles released from the T cells are obtained by isolating the extracellular vesicles released from the T cells from the medium in which the contacting of step (b) is performed.

Details and preferences with respect to the various aspects of this embodiment are already described herein elsewhere and likewise apply to this embodiment and will, for conciseness, not be repeated here.

There is also provided for a method of treatment of a subject in need thereof wherein the method comprises administering to the subject T cells obtainable by contacting isolated T cells with at least one antimitotic agent, preferably selected from the groups consisting of a taxane and a vinca alkaloid, and/or administering to the subject extracellular vesicles released from said T cells.

In some embodiments, in the method of treatment according to the invention the T cells and/or extracellular vesicles are obtained by:
(i) providing isolated T cells;
(ii) contacting the provided T cells with at least one antimitotic agent, preferably selected from the groups consisting of a taxane and a vinca alkaloid, preferably in an aqueous medium; and
(iii) obtaining the T cells contacted with the at least one antimitotic agent, and/or obtaining the extracellular vesicles released from said T cells.

In some embodiments, in the method of treatment according to the invention the extracellular vesicles are obtained by:
(i) providing isolated T cells;
(ii) contacting the provided T cells with at least one antimitotic agent, preferably selected from the groups consisting of a taxane and a vinca alkaloid, preferably in an aqueous medium; and
(iii) obtaining the T cells contacted with at least one antimitotic agent, and obtaining the extracellular vesicles released from said T cells.

In some embodiments, in the method of treatment according to the invention the T cells and/or extracellular vesicles are obtained by:
(i) providing isolated T cells;
(ii) contacting the provided T cells with at least one antimitotic agent, consisting of a vinca alkaloid, preferably in an aqueous medium; and
(iii) obtaining the T cells contacted with at least one antimitotic agent, and/or obtaining the extracellular vesicles released from said T cells.

In some embodiments, the method of treatment according to the invention is for a cancer subject and/or the treatment is in order to prevent neuropathy, preferably peripheral neuropathy, vinca alkaloid-induced neuropathy and/or taxane-induced neuropathy in a subject.

Details and preferences with respect to the various aspects of this embodiment are already described herein elsewhere and likewise apply to this embodiment and will, for conciseness, not be repeated here.

There is also provided for T cells obtained by contacting isolated T cells with at least one antimitotic agent, preferably selected from the groups consisting of a taxane and a vinca alkaloid, and/or extracellular vesicles released from said T cells.

Details and preferences with respect to the various aspects of this embodiment are already described herein elsewhere and likewise apply to this embodiment and will, for conciseness, not be repeated here.

There is also provided for extracellular vesicles released from T cells obtained by contacting isolated T cells with at least one antimitotic agent, preferably selected from the groups consisting of a taxane and a vinca alkaloid.

Details and preferences with respect to the various aspects of this embodiment are already described herein elsewhere and likewise apply to this embodiment and will, for conciseness, not be repeated here.

There is also provided for T cells obtained by contacting isolated T cells with at least one antimitotic agent consisting of a vinca alkaloid, and/or extracellular vesicles released from said T cells.

Details and preferences with respect to the various aspects of this embodiment are already described herein elsewhere and likewise apply to this embodiment and will, for conciseness, not be repeated here.

Finally, the is also provided for a method for in vitro screening compounds, wherein the method comprises
(a) providing T cells;
(b) contacting the provided T cells in vitro with a candidate compound, and
(c) measuring the effect of the candidate compound on cancer cell killing by the T cells contacted with the candidate compound.

In some embodiment, the method of screening for compounds further comprises contacting the provided T cells with a taxane and/or with a vinca alkaloid, wherein the contacting with the taxane and/or the vinca alkaloid is before, during, and/or after the contacting with the candidate compound.

The skilled person is well aware of methods that allow for measuring the effect of a candidate compound on cancer cell killing by the T cells contacted with the candidate compound, including such methods as described herein.

It will be understood that all details, embodiments, and preferences discussed with respect to one aspect of embodiment of the invention is likewise applicable to any other aspect or embodiment of the invention and that there is therefore not need to detail all such details, embodiments, and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

### Example 1

### Introduction

Together, studies on antimitotic agents, such as taxanes, indicate that alternative, mitotic-independent mechanisms of action of these antimitotic agents may exist in vivo; however, such mechanisms have not been uncovered yet.

One major difference between the in vitro and in vivo experimental settings is the presence of immune cells in the microenvironment, even in immunocompromised models of cancer where some immune functions and immature immune cells are maintained.

With this in mind, the inventors aimed to discover the mode of action of antimitotic agent-mediated killing, in particular taxanes-mediated killing and/or vinca alkaloid-mediated killing in the in vivo setting. The results show that antimitotic agents such as taxanes and vinca alkaloids can directly activate T cells to specifically eradicate tumor cells. Mechanistically, the results show that antimitotic agents such as taxanes and vinca alkaloids appear to induce a release of cytotoxic extracellular vesicles by T cells that subsequently trigger tumor death. The data also shows that T cells pre-treated with taxanes ex vivo have an anti-tumor capacity once transplanted in vivo. These findings establish a novel paradigm for the mode of action of antimitotic agents such as taxanes and vinca alkaloids, two of the most widely used chemotherapies for cancer treatment.

The study also enables a novel therapeutic approach to exploit an optimal T cell-dependent anti-tumor activity of antimitotic agents such as taxanes and vinca alkaloids while avoiding systemic exposure to their toxicity. Details of the study are described in the following examples.

### Example 2

### Methods

### Organoid Culture and Treatment

Breast cancer organoids were derived from K14-Cre; Brca1fl/fl; p53fl/fl mouse mammary tumors (KB1P (Kester, L., et al. Clin Cancer Res, 2022.)) and from MMTV-PyMT (Duarte, A. A., et al. Nat Methods, 2018. 15(2): p. 134-140) mouse mammary tumors. Both lines were cultured in 50 μl drops of Cultrex PathClear Reduced Growth Factor Basement Membrane Extract Type 2 (BME, Amsbio, 3533-005-02). KB1P organoids were cultured in Advanced Dulbecco's modified Eagle's medium (adDMEM/F12; Thermo Fisher Scientific. Cat. No. 12634-010) containing Hepes (10 mmol/L; Thermo Fisher Scientific, Cat. No. 15630-056), Penicillin/streptomycin (10,000 U/ml; Thermo Fisher Scientific, Cat. No. 15140-122), B27 (2% Thermo Fisher Scientific, Cat. No. 17504-044), N-acetylcysteine (1.25 mM; Sigma-Aldrich, Cat. No. A9165) and fibroblast growth factor (FGF, 12 ng/ml; Invitrogen, Cat. No. PHG0261). MMTV-PyMT organoids were cultured in DMEM/F12 GlutaMAX (Gibco, Cat. No 10565018) supplemented with 10 mmol/L Hepes (Gibco, Cat. No 15630106), Penicillin/streptomycin (10,000 U/ml; Thermo Fisher Scientific, Cat. No. 15140-122), B27 (2% Thermo Fisher Scientific, Cat. No. 17504-044) and fibroblast growth factor (FGF, 12 ng/ml; Invitrogen, Cat. No. PHG0261). Organoids were kept in 20% O2, 5% CO2 and at 37 degrees Celsius. Organoid cultures were routinely negatively tested for mycoplasma using the MycoAlert PLUS kit (Lonza, Cat. No. LT07-118). For passaging, organoids were split using TrypLE Express (Gibco, Cat. No. 12605010) while shaking for 10-15 min at 900 rpm and 37 degrees Celsius For in vitro treatment, organoids were treated with doxorubicin (5 nmol/L); docetaxel (5 nmol/L) and cyclophosphamide (5 μmol/L).

### Generation and Culture of Normal Mouse Mammary Epithelial Cells (MECs)

MECs were isolated from 12- to 15-week-old female Friend Virus B mice, as previously described (Ooft, S. N., et al. Sci Transl Med, 2019. 11(513)). This matches the age of FVB mice from which tumors were isolated for preparing tumor organoids. MEC were cultured in DMEM/F12 (Gibco, Cat. No. 10565-018) containing fetal bovine serum (FBS, 10%), Penicillin/streptomycin (10,000 U/ml; Thermo Fisher Scientific, Cat. No. 15140-122), epidermal growth factor (EGF, 5 ng/ml; Peprotech, Cat. No. AF-100-15), insulin (5 ng/ml; Sigma, Cat. No. IO516-5ML) and cholera toxin (Gentaur, Cat. No.). MECs were kept in 20% O2, 5% CO2 and at 37 degrees Celsius.

### Mice

Experiments were approved by the Animal Welfare Committee of the Netherlands Cancer Institute and were performed in accordance with national guidelines. Mice were housed in individually ventilated cage (IVC) systems under specific pathogen-free conditions and received chow and water ad libitum. FVB/NRj females (Janvier), C57BL/6-Tg (TcraTcrb) 1100Mjb/J females (OT I mice, Jackson laboratory), and ROSA26mTmG FVB females (NKI) were used at 8-20 weeks of age at the time of fat pad injection of tumor organoids or spleen isolation.

### In Vivo Transplantation of Tumor Organoids and Monitoring of Tumor Growth

For tumor generation, tumor organoids were transplanted into the 4th mammary fat pad of FVB female mice. Rimadyl (0.067 mg/ml, Zoetis) was administered to mice in the drinking water from 24 h before surgery and for 48 h after surgery. Mice were also treated with temgesic (0.1 mg/kg, Indivior Europe Limited), 1 hr before and 12 h after surgery. Mice were anesthetized with isoflurane (2%, v/v) and their eyes were covered with duratears (Alcon). About 100,000 single cells derived from KB1P organoids or 200,000 single cells derived from MMTV-PyMT organoids were injected in 30 μl of BME (Amsbio, Cat. No. 3533-005-02). Mice were weighed and monitored three times a week after transplantation of tumor organoids. Tumor volume was measured with calipers three times weekly. The researcher performing the measurement of tumor volume was blinded to the treatment group. For survival experiments, endpoint was reached when tumor reached 2000 mm3. For specific experiments, reference is made to the schematic representations of treatment timelines depicted in the figures.

### Drug Treatment In Vivo

Once tumors were established and reached a size of ˜7×7 mm, mice were randomized into treatment groups with the following drug dosages and timeline:
AC+T treatment: every 14 days, cyclophosphamide (144 mg/kg, administered via intraperitoneal injection), doxorubicin (5 mg/kg, administered via intravenous injection) and docetaxel (25 mg/kg, resuspended in acetonitrile containing 0.1% acetic acid, administered via intravenous injection). Doxorubicin hydrochloride (Adriamycin) and cyclophosphamide (C), when combined in a treatment, are herein referred to as “AC”. Taxanes, as broadly described and disclosed herein, are referred to as “T”. In one example, said taxane is docetaxel.
AC treatment: every 14 days, cyclophosphamide (144 mg/kg, administered via intraperitoneal injection) and doxorubicin (5 mg/kg administered via intravenous injection).
Docetaxel treatment: every 14 days, docetaxel (25 mg/kg, re-suspended in acetonitrile containing 0.1% acetic acid and administered via intravenous injections).
Vehicle control for chemotherapy: every 14 days, the same volume of the vehicle as the corresponding chemotherapy was administered to mice. Vehicle for cyclophosphamide: saline; vehicle for doxorubicin: saline; vehicle for docetaxel: acetonitrile containing 0.1% acetic acid.
IgG treatment: on day 1, 3 and 5 prior to treatment with control or with chemotherapy, treatment with 400 μg (day 1), or 200 μg (day 3 and day 5) per mouse with InVivoMAb rat IgG2b isotype control (BioXCell, clone LTF-2, Cat. No. BE0090).
αCD4 treatment: on day 1, 3 and 5 prior to treatment with control or with chemotherapy, treatment with 400 μg (day 1), or 200 μg (day 3 and day 5) per mouse with InVivoMAb anti-mouse CD4 (BioXCell, clone GK1.5, Cat. No. BE003-1).
αCD8 treatment: on day 1, 3 and 5 prior to treatment with vehicle or with chemotherapy, treatment with 400 μg (day 1), or 200 μg (day 3 and day 5) per mouse with InVivoMAb anti-mouse CD8 (BioXCell, clone YTS 169.4, Cat. No. BP0117).

### Flow Cytometry Isolation of T Cells

Preparation of tissues: For isolation of T cells from mammary tumors, tumors were isolated, adjacent lymph nodes were removed and samples were mechanically dissociated using scalpel blades. Subsequently, enzymatic digestion of the tissue was performed in a mix containing collagenase A (3 mg/ml, Sigma Aldrich, Product No. 10103578001) and DNAse (25 μg/ml, Sigma Aldrich, Product No. 10104159001), diluted in DMEM/F12 GlutaMAX (Gibco, Cat. No 10565018) containing FBS (10%), at 37 degrees Celsius for 1 hr in a rotating incubator. Samples were next filtered on a 70-μm cell strainer (Corning), washed in DMEM/F12 containing FBS (10%) and in FACS buffer (PBS, 5% FBS and 5 mM EDTA). For isolation of T cells from the spleen of FVB female mice, aged 8- to 20-weeks old, the tissue was gently mechanically dissociated using a scalpel blade and glass slides.

Staining protocol: For both mammary tumors and spleen tissues, samples were next incubated in a red cell lysis buffer for 5 min at room temperature (NH4Cl 155 mmol/L, KHCO3 1 mmol/L, 0.1 mmol/L EDTA diluted in MilliQ water, pH=7.4). Subsequently, samples were blocked for 5 min at room temperature in a blocking buffer (FACS buffer containing 1:50 Fc block CD16/CD32 (BD Biosciences, Clone 2.4G2, Cat. No. 553141)). Next, samples were incubated with primary antibodies in the dark for 45 min at 4° C. (anti-CD11b-BV650 (1:1200, BD Bioscience, clone M1/70, Cat. No. 563402); anti-CD4-PE (1:300, ThermoFisher, clone GK1.5, Cat. No. 12-0041-82) or anti-CD4-eFluor660 (1:300, BD Bioscience, clone GK1.5, Cat. No. 50-0041-82); anti-CD8-eFluor660 (1:100, ThermoFisher, clone 53-6.7, Cat. No. 50-0081-82)). Samples were sorted on a Fusion Cell Sorter (Becton Dickinson). Gating strategies are depicted on FIGS. 7 and 9.

### Mouse T Cell:Organoid Co-Culture Experiments

Preparation of T cells: T cells isolated from mouse spleens or from mouse mammary tumors were cultured in Roswell Park Memorial Institute (RPMI) 1640 Medium (Thermo Fisher Scientific, Cat. No. 21875034) containing FBS (10%, Thermo Fisher Scientific, Cat. No. A4766801), Penicillin/streptomycin (10,000 U/ml; Thermo Fisher Scientific, Cat. No. 15140-122), IL2 (Thermo Fisher Scientific, Cat. No. PMC0025), β-mercaptoethanol (50 μmol/L) and Ultraglutamine (2 mmol/L, Lonza, Cat. No. BE17-605E/U1). T cells were kept in 20% O2, 5% CO2 and at 37 degrees Celsius

In order to assess the direct effects of docetaxel on T cell activation for T cells isolated from the spleen, T cells were cultured in the absence of CD3 and CD28 antibodies.

For T cells isolated from mouse mammary tumors, T cells were directly co-cultured with tumor organoids after FACS isolation. For T cells isolated from the spleen, T cells were treated for 72 h with PBS or with docetaxel (docetaxel 10 nmol/L), in the presence or absence of an LCKi (10 μmol/L, Merck Millipore, Cat. No. 213743-31-8). To evaluate the docetaxel-specificity of our observations, T cells were treated with paclitaxel 10 nmol/L or carboplatin 50 μmol/L for 72 h. Subsequently, T cells were collected and washed 3 times in PBS to remove residual chemotherapy or LCKi.

### Preparation of Organoids:

Tumor organoids or MECs were gently dissociated in TrypLE (Thermo Fisher Scientific, Cat. No. 12605-010) for 10 min at 37 degrees Celsius into small organoids (3-5 cells per organoids). Organoids and MECs were plated in 5 μl of BME in a 96-well plate.

T cells were added with a 1:3 ratio (T cell: tumor cell/MEC) in cell number. Cells were co-cultured in a 1:1 mix of T cell medium: organoid/MEC medium for 72 h.

For treatment of organoids with conditioned medium generated by T cells, T cells were pre-treated with PBS or with docetaxel (docetaxel 10 nmol/L) for 72 h. T cells were removed from the conditioned medium by centrifugation and the freshly isolated conditioned media were mixed with organoid medium (1:1 ratio) and added to the organoids for 72 h.

### Jurkat:Human Organoids and Cell Co-Culture Experiments

Preparation of Jurkat T cells: Jurkat T cells were cultured in Roswell Park Memorial Institute (RPMI) 1640 Medium (Thermo Fisher Scientific, Cat. No. 21875034) containing FBS (10%, Thermo Fisher Scientific, Cat. No. A4766801), Penicillin/streptomycin (10,000 U/ml; Thermo Fisher Scientific, Cat. No. 15140-122), and Ultraglutamine (2 mmol/L, Lonza, Cat. No. BE17-605E/U1). Jurkat T cells were kept in 20% O2, 5% CO2 and at 37 degrees Celsius. Jurkat T cells were treated for 72 h with PBS or with docetaxel (docetaxel 5 nmol/L). Subsequently, Jurkat T cells were collected and washed 3 times in PBS to remove residual chemotherapy.

### Preparation of Cell Lines:

MDA-MB-231, MCF7, A549, A375, U251-MG, EGI-1, HuH7, HEK293T and RKO cells were grown in DMEM GlutaMAX (Gibco, Cat. No. 31966047) containing FBS (10%, Thermo Fisher Scientific, Cat. No. A4766801) and Penicillin/streptomycin (10,000 U/ml; Thermo Fisher Scientific, Cat. No. 15140-122). Hep-1 cells were grown in Roswell Park Memorial Institute (RPMI) 1640 Medium (Thermo Fisher Scientific, Cat. No. 21875034) containing FBS (10%, Thermo Fisher Scientific, Cat. No. A4766801) and Penicillin/streptomycin (10,000 U/ml; Thermo Fisher Scientific, Cat. No. 15140-122). RPE cells were cultured in DMEM/F12 GlutaMAX (Gibco, Cat. No 10565018) containing FBS (10%, Thermo Fisher Scientific, Cat. No. A4766801), Penicillin/streptomycin (10,000 U/ml; Thermo Fisher Scientific, Cat. No. 15140-122), and Ultraglutamine (1%, Lonza, Cat. No. BE17-605E/U1). Cells were kept in 20% O2, 5% CO2 and at 37 degrees Celsius and passaged ˜twice a week using TrypLE (Thermo Fisher Scientific, Cat. No. 12605-010). Cells were routinely negatively tested for mycoplasma using the MycoAlert PLUS kit (Lonza, Cat. No. LT07-118).

### Preparation of Organoids:

Breast cancer patient-derived organoids and milk-derived organoids were generated and cultured as previously described (Montano, M., Model systems, in Translational Biology in Medicine. 2014).

Jurkat T cells were added with a 2:1 ratio (T cell: tumor or healthy cell) in cell number. Organoids and cells were co-cultured with Jurkat T cells in a 1:1 mix of cancer/healthy organoids/cell medium: Jurkat cell medium for 72 h.

### MTT Assessment of Mouse Splenic T Cell and Jurkat T Cell Survival Following Chemotherapy Treatment In Vitro

About 100,000 T cells were seeded per well in a 96-well plate and treated with

increasing doses of docetaxel, paclitaxel, or carboplatin for 72 h. Next, T cells were incubated with Thiazolyl Blue Tetrazolium Bromide (0.5 mg/ml, Sigma Aldrich, Cat. No. 298-93-1) for 3 hr in the dark at 37 degrees Celsius. Subsequently, T cells were incubated for 16 h in the dark in a lysis buffer (40% dimerhylofrmamide, 0.55 mol/L sodium dodecyl sulfate, 2% acetic acid, 8.4 mmol/L HCl in MilliQ water). Plates were read with a 565 nm wavelength.

### MTT Assessment of Organoid Survival

Following co-culture, medium was removed, and cultures were washed in PBS to remove T cells. Organoids were incubated with Thiazolyl Blue Tetrazolium Bromide (0.5 mg/ml, Sigma Aldrich, Cat. No. 298-93-1) for 3 hr in the dark at 37 degrees Celsius. Subsequently, organoids were incubated for 16 h in the dark in a lysis buffer (40% dimerhylofrmamide, 0.55 mol/L sodium dodecyl sulfate, 2% acetic acid, 8.4 mmol/L HCl in MilliQ water). Plates were read with a 565 nm wavelength.

### Immunofluorescence Staining and Microscopy.

Organoids were fixed in 4% paraformaldehyde in PBS for 20 min at room temperature, followed by permeabilization in 0.2% TritonX-100 in PBS for 15 min at room temperature. A block was next performed in 5% BSA/PBS for 2 h at room temperature, followed by staining overnight at 4 degrees Celsius with anti-cleaved caspase 3 (1:200, Asp175, Cell Signalling, Cat. No. 9661). Appropriate Alexa Fluor labelled secondary antibody (Thermo Fisher Scientific) was combined with DAPI (1 μg/ml) and incubated for 2 hr in the dark at room temperature.

Images were acquired on an inverted Leica TCS SP5 confocal microscope (Mannheim, Germany), in 8 bit with a 20× dry immersion objective (HCX PL APO CS 20.0×0.70 DRY UV)). Fiji was used for quantification.

### Experiments Using Lung Cancer Patient-Derived Material

### Generation of Tumor Organoids Reactive PBMC

Tumor-reactive patient T cells were generated by co-culturing PBMCs and autologous tumor organoids as described previously (Dekkers, J. F., et al. Nat Protoc, 2021. 16(4): p. 1936-1965; Cattaneo, C. M., et al. Nat Protoc, 2020. 15(1): p. 15-39). In brief, following incubation with 200 ng/ml IFNγ for 24 hr, patient tumor organoids were dissociated into single cell suspensions using TripLE Express. Tumor organoid cells were mixed with patient PBMCs (20:1 tumor cell/PBMC ratio) and 105 cells were seeded in each well of a U-bottom 96-well plate pre-coated with 5 μg/ml αCD28 antibody. Co-culture medium consisted of RPMI 1640 supplemented with 2 mmol/L Ultraglutamine I, penicillin/streptomycin, 10% human AB serum, 150 U/ml rh-IL-2 and 20 μg/ml αPD1 blocking antibody (Merus, The Netherlands). Medium, IL-2 and αPD1 were refreshed every 2-3 days. PBMCs were harvested and re-stimulated every 7 days by re-plating with fresh tumor organoid cells. After two weeks of co-culture, PBMCs were harvested and used for downstream analysis or cryopreserved for later use.

### Tumor Recognition Assay

Tumor-reactive PBMC were thawed in pre-warmed T cells medium and incubated for 15 minutes with 25 U/mL Benzonase. After washing, cells were re-suspended at 2−3×106 cells/ml in T cell medium and cultured overnight at 37° C. T cell medium consisted of RPMI 1640 supplemented with 2 mmol/L Ultraglutamine I, penicillin/streptomycin, 10% human AB serum and 150 U/ml rh-IL-2. The day after, PBMC were incubated with (or without) 10 nmol/L docetaxel for 48 h.

For evaluation of tumor reactivity, 105 PBMCs were re-stimulated with autologous tumor organoids or autologous healthy organoids (isolated from Geltrex for 48 h and stimulated with 200 ng/ml IFNγ for 24 h, as described in (Dekkers, J. F., et al. Nat Protoc, 2021. 16(4): p. 1936-1965; Cattaneo, C. M., et al. Nat Protoc, 2020. 15(1): p. 15-39; Voorwerk, L., et al. Nat Med, 2019. 25(6): p. 920-928) at a 2:1 effector: target ratio and seeded in αCD28 coated plates in the presence of 20 μg/ml αPD1 and co-cultured for 24 hours. At the end of the 24 h, 50 μl of the assay supernatant were taken on the side and used for the quantification of the secreted IFNγ. To this purpose, the Human IFN-γ Flex Set (BD bioscience) was used in accordance with the manufacturer's instructions. For the evaluation of CD137 expression, at the end of the 24 h, cells were washed twice in FACS buffer and stained with the following antibodies: αCD3 PerCPCy5.5, αCD4 FITC, αCD8 BV421, αCD137 APC, and near IR viability dye for 30 minutes at 4° C. in the dark. PBMCs stimulated with 50 ng/ml PMA (Sigma Aldrich) and 1 μg/ml ionomycin (SigmaAldrich) served as positive controls and PBMCs cultured without tumor stimulation as negative controls. Cells were then washed twice with FACS buffer and recorded with a Becton Dickinson Fortessa or LSRII flow cytometer.

### Tumor and Healthy Organoids Killing Assay.

To express luciferase in the tumor organoids, we used pLenti CMV Puro LUC (w168-1; Plasmid #17477; Addgene). Tumor-reactive PBMC were thawed in pre-warmed T cell medium and incubated for 15 minutes with 25 U/ml Benzonase. After washing, cells were re-suspended at 2−3×10\hat{ }6 cells/ml in T cell medium and cultured overnight at 37° C. The day after, PBMC were incubated with (or without) 10 nmol/L docetaxel for 48 hr. To determine the sensitivity of tumor and healthy organoids to T cell-mediated killing, flatbottom non-tissue culture treated plates were coated with 5 μg/ml αCD28 and kept at 4° C. overnight prior to co-culture. Tumor organoids were previously transduced with luciferase reporter gene. Organoids were isolated from Geltrex 48 hours prior to co-culture and stimulated with 200 ng/ml IFNγ for 24 hr prior to co-culture. The next day, part of the organoids was dissociated to single cells and counted using a hemocytometer. This was used to infer the number of tumor cells per tumor organoid to allow co-culture of organoids and T cells at a 5:1 effector:target ratio. Next, organoids and tumor reactive T cells were resuspended in the T cell medium. αCD28 coated plates were washed twice with PBS and organoids were seeded for 72 hr in triplicate without T cells or with 5×10⁴ autologous tumor-reactive T cells (incubated or not with docetaxel). At the end of the 72 hours, tumor and healthy organoids viability in the different conditions was evaluated by microscopy, taking bright-field pictures at 5 and 10× magnification. Tumor cell viability in the different conditions was also measured by luciferase reporter assay using 3 μg/mL luciferin. Luminescence was measured with a Tecan reader (1,000 ms exposure).

### Immunohistochemistry

Tumors were collected and fixed in EAF (ethanol/acetic acid/formaldehyde/saline at 40:5:10:45 v/v) and embedded in paraffin. Next, 4 μm-thick sections were stained with CD4 (eBiosciences, Cat. No. 14_9766_80) or CD8 (eBiosciences, Cat. No. 14-0808). The stained sections were reviewed with a Zeiss Axioskop2 Plus microscope (Carl Zeiss microscopy, Jena, Germany).

### Quantification of Cells With a Mitotic Plate

Cells with a mitotic plate were identified on Hematoxylin and Eosin-stained sections, based on nuclear condensation and cellular shape, as previously performed (Lisci, M., et al., Science, 2021. 374; Conway, J. R. W., et al., Cell Rep, 2018. 23(11): p. 3312-3326; Nielsen, P. S., et al. Diagn Pathol. 2016. 11: p. 35). For each tumor, 10 fields of view (FOV) were analyzed and the number of cells with a mitotic plate per FOV was plotted.

### Time-Lapse Microscopy of Co-Cultures

T cells and KB1P organoids were co-cultured as described above. Images were acquired on a Leica SP5 confocal microscope (Mannheim, Germany) in 12-bit with a 20× dry immersion objective (HCX PL APO CS 20.0×0.70 DRY UV). Time-lapse imaging was analyzed using Zeiss software and ImageJ software.

### Mass-Spectrometry

T cells isolated from the mouse spleen were treated with PBS or docetaxel (10 nmol/L) for 72 h, in the absence of FBS. Next, conditioned media were collected, and T cells were removed by centrifugation. To analyze the proteins contained in EVs, EVs were isolated using Sepharose beads as indicated below. For each repeat, we isolated the EVs released by 6×10\hat{ }6 T cells treated with PBS or with docetaxel. Conditioned media or EVs were processed for mass-spectrometry as follows. Proteins in the conditioned media were precipitated with subsequent addition of 4× (v/v) MeOH, 1× (v/v) CHCl3 and 3× (v/v) MQ. The protein pellet was resuspended in digestion buffer, containing 1% SDC, 100 mmol/L Tris pH 8.5, 5 mmol/L Tris (20carboxyethyl)phosphine (TCEP) and 30 mmol/L CAA. Trypsin (1:50) and LysC (1:100) were added and proteins were digested overnight at 37degrees Celsius. Digestion was terminated and SDC was precipitated by addition of 10% FA to a final concentration of 0.5% FA (v/v). SDC was removed by centrifugation. Peptides were desalted using Oasis HLB plates (Waters).

### LC-MS/MS

Microflow LC-MS/MS was performed using an Ultimate 3000 (Thermo Fisher Scientific, Bremen, Germany) coupled to an Orbitrap Exploris™ 480. Lyophilized phosphopeptides were resuspended in 1% (v/v) formic acid and injected, trapped, and washed on a trap-column (μ-Precolumn, 300 μm i.d.×5 mmC18 PepMap100, 5 μm, 100 Å (Thermo Scientific, P/N 160454)) for 5 min at a flow rate of 5 μL/minute with 92% buffer A (0.1 FA, in HPLC grade water). Peptides were subsequently transferred onto an analytical column (75 μm×50 cm Poroshell 120 EC-C18, 2.7 μm, Agilent Technology, packed in-house) and separated at 40° C. at a flow rate of 0.3 μl/min using a 175 min linear gradient from 9% to 36% buffer B (0.1% FA, 80% ACN). Electrospray ionization was performed using 1.9 kV spray voltage and a capillary temperature of 275° C. The mass spectrometer was operated in data-dependent acquisition mode: full scan MS spectra (m/z 375-1,600) were acquired in the Orbitrap at 60,000 resolution for a maximum injection time set to auto-mode with a standard AGC target. High resolution HCD MS2 spectra were generated using a normalized collision energy of 28%. MS2 scans were acquired in the Orbitrap mass analyzer at a resolution of 30,000 (isolation window of 1.4 Th) with a standard AGC target and an automatic maximum injection time. Precursor ions with unassigned charge state as well as charge state of 1+ or superior/equal to 6+ were excluded from fragmentation.

### Data Analysis

Raw files were processed using MaxQuant software (version 1.6. 17.0) and the Andromeda search engine was used to search against Mus Musculus database (Uniprot reviewed, 17090 entries) with the following parameters: trypsin digestion with a maximum of 2 missed cleavages, carbamidomethylation of cysteines (57.02 Da) as a fixed modification and methionine oxidation (15.99 Da) as variable modification. Mass tolerance was set to 4.5 ppm at the MS1 level and 20 ppm at the MS2 level. The False Discovery Rate (FDR) was set to 1% for peptide-spectrum matches (PSMs) and protein identification using a target-decoy approach, minimum peptide length was set to 7 residues. Relative label-free quantification was performed using the MaxLFQ algorithm with the minimum ratio count set to 2. LFQ intensities were log2 transformed and only proteins with at least two valid values in each condition were considered. Relative label-free quantification was performed using iBAQ algorithm, intensities were log2 transformed and only proteins with at least two valid values were considered.

### Isolation of Extracellular Vesicles and Soluble Fraction and Treatment of Tumor Organoids In Vitro

T cells isolated from the mouse spleen were treated with PBS or docetaxel (10 nmol/L) for 72 h, in the absence of FBS. Next, conditioned media were collected, and T cells were removed by centrifugation.

For isolation of extracellular vesicles (EVs), Sepharose CL-2B (GE Healthcare, 17-0140-01) was first washed with a citrate buffer (0.32% citrate in PBS, pH=7.4). Next, columns were prepared by adding a nylon panty hose into the outlet of a syringe. Sepharose was next stacked into the column and allowed to settle until 10 ml of stacked column was reached. Subsequently, 1.5 ml of conditioned medium was carefully added to the column and sample collection of fractions of 0.5 ml started immediately. When the conditioned medium had entered the Sepharose, a few drops of buffer were added progressively. Once this buffer had entered the Sepharose, buffer was added until filling the column entirely. Buffer was continuously added during the collection of all fractions. Fractions 9 and 10 contained the majority of EVs with a high purity, fractions 20 and 21 contain the majority of proteins and high-density lipoproteins. Next, collected fractions were loaded onto Amicon Ultra 2 ml columns with a 10 kDa cut off (Merck UFC201024). Fractions were centrifugated at 3,000 g for at least 10 min and samples were recovered by placing the filter unit upside down and centrifugating at 1,000 g for 2 min. Following isolation, EVs were kept in −80° C. conditions until functional assays. To evaluate the killing ability of EVs, tumor organoids were seeded on a glass-bottom 96-well plate (Greiner-BioOne, Cat. No. 655076) in 5 μl of BME. Organoids were treated with EVs or with the SF at a 1:10 dilution in organoid medium. 72 h after the initiation of treatment, organoids were washed and fixed for immunohistochemical assessment of cleaved-caspase 3.

### Electron Microscopy and NanoSight Particle Analysis

For electron microscopy measurements, EV fractions were spotted on carbon/formvar-coated mesh grids. After blotting off the excess liquid, the samples were contrasted by 2% uranylacetate (Polysciences Inc, Cat. No. 21447-25) in water for 1 minute, the excess stain is blotted off and grids was air dried. Vesicular structures were imaged in a 60 KV JEOL1010 (JEOL) TEM at 60,000× magnification using a 4 k×2.6 k pixel CCD side-mounted camera (Modera, EMsis GmbH).

The distribution of EV diameter was analyzed using a NanoSight. The measurements report the tracking of Brownian motion of individual nanoparticles detected by scattered light, as a function of temperature and dispersing medium viscosity.

### EV-Labelling With PKH67 and Uptake Experiment

EVs were labelled with the green fluorescent membrane-linker dye, PKH67 (Sigma, USA), as per the manufacturer's instructions. PKH67-labelled EVs were incubated at a 1:10 dilution (in line with our killing assays described above) with either MEC, KB1P or MMTV-PyMT organoids seeded in 5 μl BME on a glass bottom culture dish (Greiner-BioOne, Cat. No. 655076). Organoids were imaged 1 h after incubation with EVs on a Leica TCS SP5 confocal microscope (Mannheim, Germany), in 8 bit with a 63× oil objective. Fiji was used for quantification.

### Transfer of T Cells in Mice Bearing Tumors

CD4 T cells were isolated from the spleen of healthy FVB female mice, based on DAPI−/CD11b−/CD4+ expression. T cells were treated in vitro with PBS or docetaxel (docetaxel 10 nmol/L) for 72 h. Subsequently, T cells were washed three times in PBS to remove residual docetaxel and 2×10\hat{ }6 T cells resuspended in 100 μl HBSS were transferred by intravenous injections into tumor-bearing mice. Importantly, tumors at the time of intravenous transfer of T cells had a size between 100 mm3 and 250 mm3. Transfer of docetaxel-treated T cells into mice bearing larger tumors (>400 mm3) led to mouse death shortly after T cell transfer, potentially due to important cytokine storm upon tumor cell killing. Histopathological analysis did not reveal significant tissue damage in those mice.

### Statistical Analysis

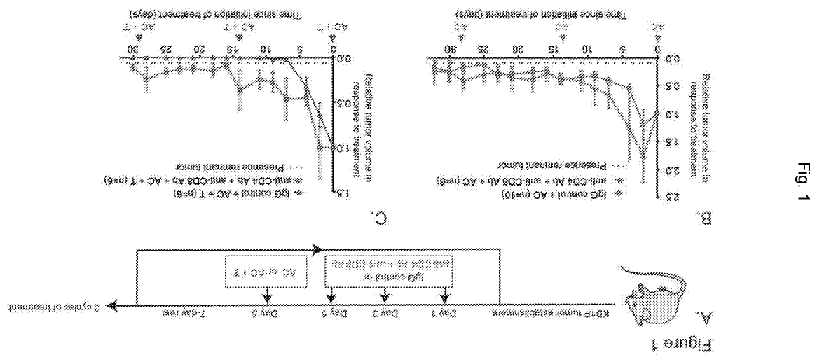

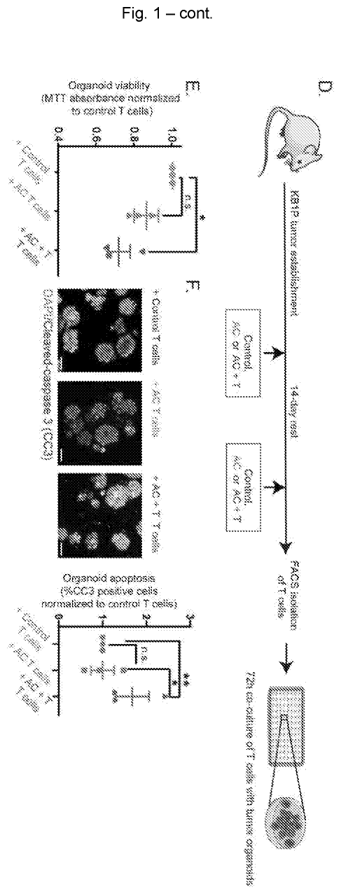

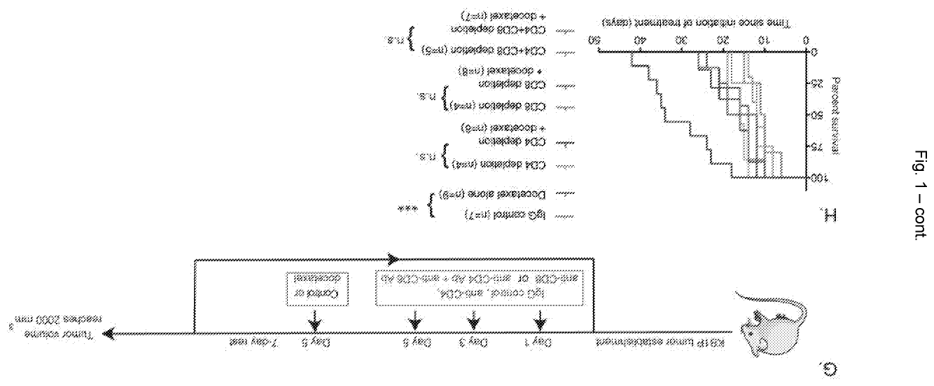

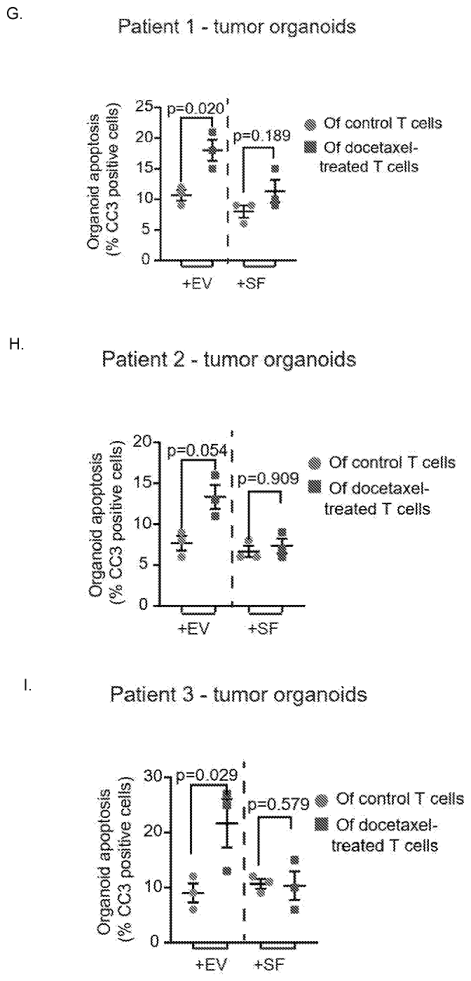

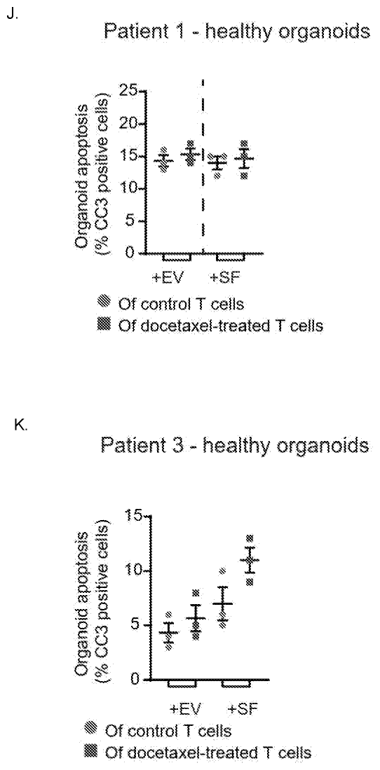

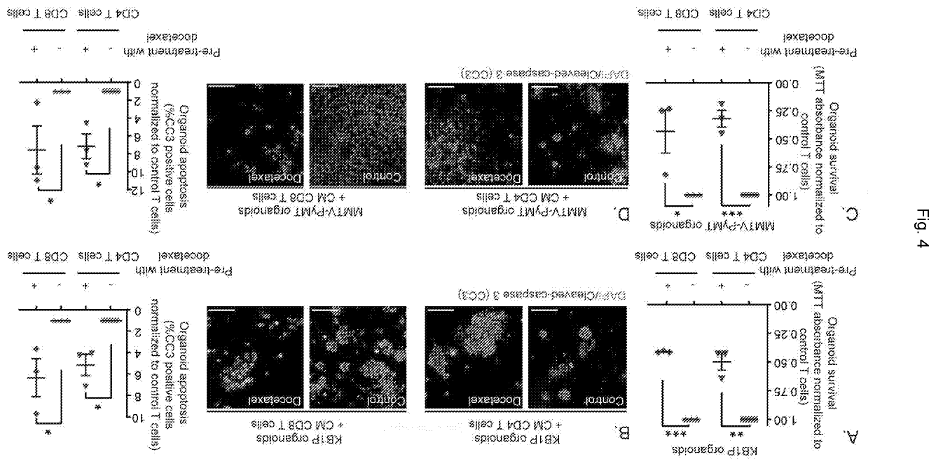

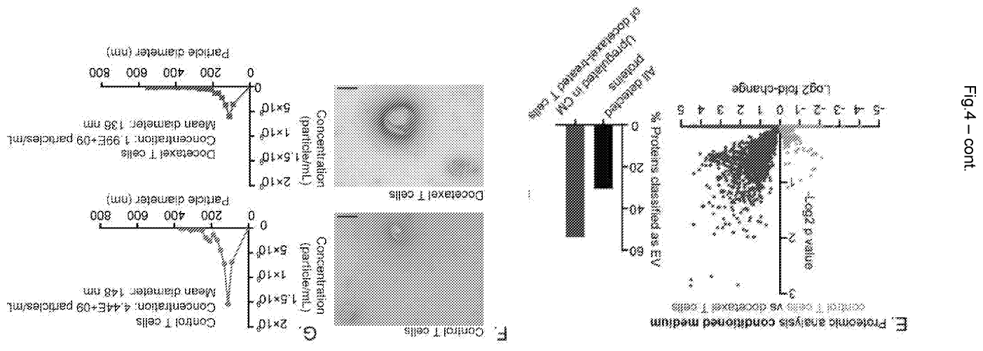

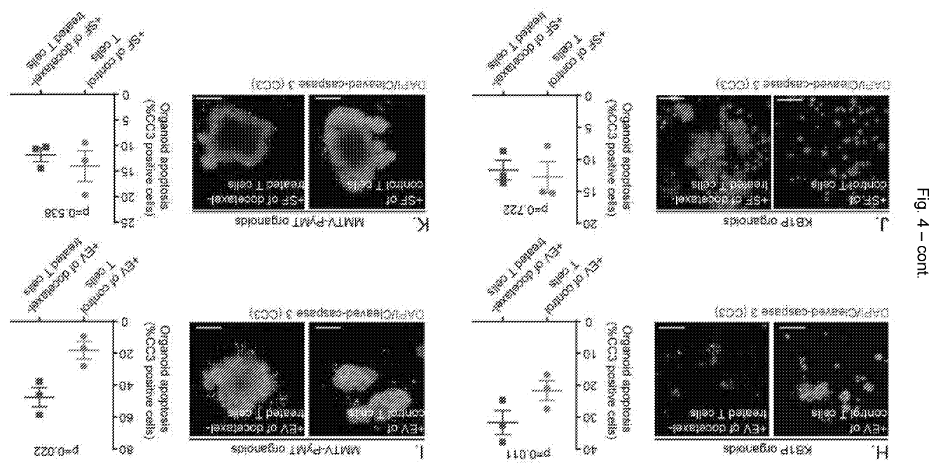

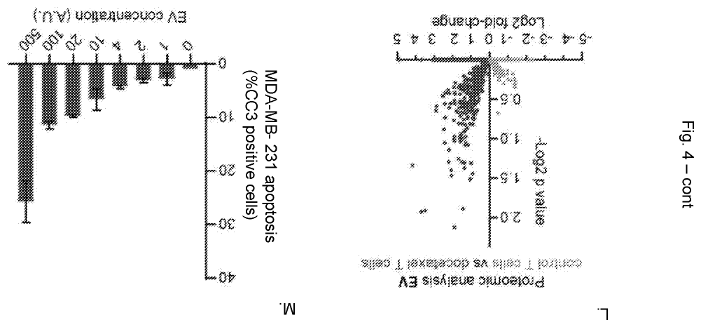

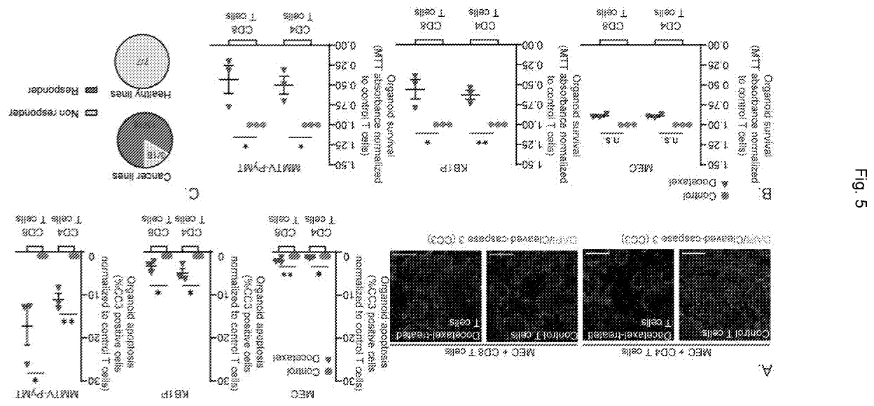

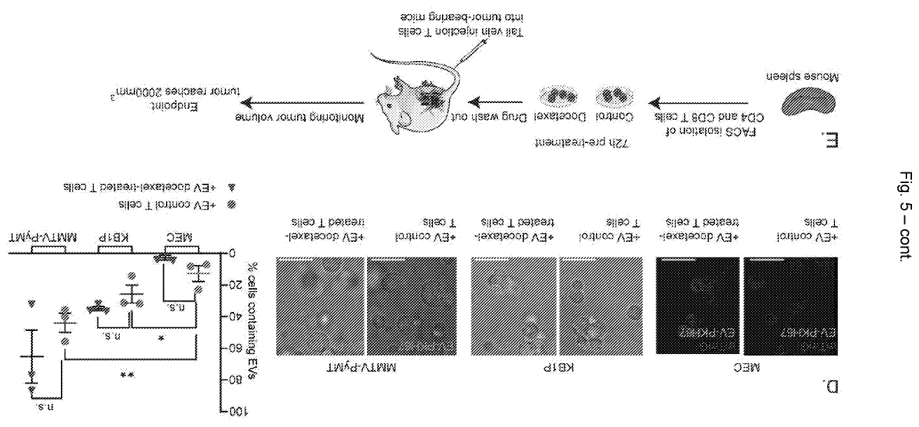

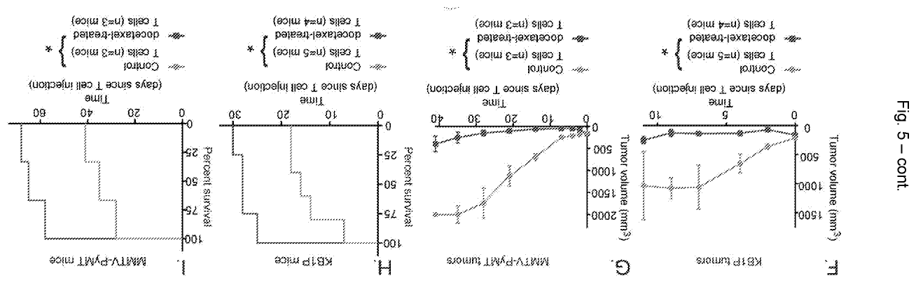

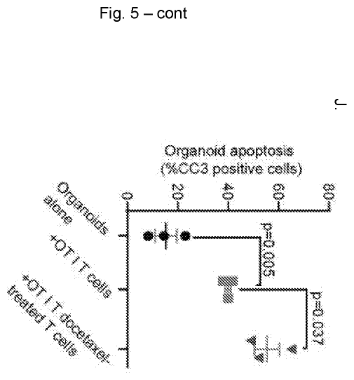

p-values were determined using unpaired, nonparametric t-test with a Mann-Whitney U correction in GraphPad Prism. Kaplan Meier survival curves were analyzed with a log-rank Mantel-Cox test in GraphPad Prism. Statistical analysis of tumor growth depicted in FIG. 5G was performed in Prism GraphPad using a mixed-effects analysis with a Geisser-Greenhouse correction. Statistical analyses of tumor growth curves depicted in FIG. 1B, C and FIG. 5F were performed in R using a linear mixed-effects model analysis.

### Generation and Culture of 2D Cell Lines From Mouse Tumor Organoids

KB1P and MMTV-PyMT 2D cell lines were generated by washing the BME out and dissociating tumor organoids in TrypLE Express (Thermo Fisher Scientific, Cat. No. 12605010) while shaking for 20 min at 37° C. Cells were next plated in 2D on plastic-bottom plates and were cultured in Dulbecco's modified Eagle's medium (DMEM, Thermo Fisher Scientific, Cat. No. 10564011) supplemented with FBS (10%, Thermo Fisher Scientific, Cat. No. A4766801), 100 U/mL penicillin (Thermo Fisher Scientific, Cat. No. 15140122), Penicillin/streptomycin (1%, Thermo Fisher Scientific, Cat. No. 15140-122), EGF (5 ng/ml, Thermo Fisher Scientific, Cat. No. 53003018) and Insulin (5 μg/ml, Sigma Aldrich, Cat. No. 10516) in 20% O2, 5% CO2 and at 37° C. Cells were kept in culture for a minimum of 10 days after being plated on plastic before functional assays, to remove cells that die during this process. 2D cells that were re-transferred into a 3D BME environment were kept in culture for the same duration before performing functional assays.

### Generation of the Hela ABCB1 Cells

Hela cells (obtained from the Medema Lab at the Netherlands Cancer Institute) were transduced with a lentiviral overexpression construct of the ABCB1 ORF. The HeLa ABCB1 cell line was derived by subsequent Taxol selection (100 nmol/L of Taxol).

### Virus Production and Transduction of Organoids to Express OVA.

For lentivirus production, 80% confluent human embryonic kidney (HEK) 293T cells were used in 10 cm dish (Greiner, Cat. No. 664160). Per dish, 7.5 μg of psPAX2, 2.5 μg PMD2.G and 10 μg of the plv-OVA-mPlum construct (gift from the Peeper lab at the Netherlands Cancer Institute) were mixed in 1 mL Opti-MEM (Thermo Fisher Scientific, Cat. No. 31985070). 40 L lipofectamine 2000 (Thermo Fisher Scientific, Cat. No. 11668019) were added to the plasmid mix and incubated at room temperature for 15 min before being added to the HEK 293T cells. 18 h later, the medium was refreshed with DMEM GlutaMAX (Thermo Fisher Scientific, Cat. No. 31966047) supplemented with streptomycin/penicillin (1%, Thermo Fisher Scientific, Cat. No. 15140122). After 48 h, the medium was collected and filtered through a 0.22 μm filter (Millipore, Cat. No. SLGS033SS). Filtered medium was concentrated with an Amilcon Ultra-15 10 k column (Millipore, Cat. No. UFC905024) for 1 h at 4,000 g. Breast cancer organoids isolated from a fully developed tumor in a MMTV-PyMT mouse with a C57BL/6NRj background were trypsinized into smaller clusters and incubated with 250 μL virus, 100 μg/mL polybrene (Sigma Aldrich, Cat. No. TR-1003-G) and 10 μmol/L Y-27632 (Bio Connect, Cat. No. S1049). Spin infection was done at 36° C., 600 g for 1 h and organoids were subsequently incubated at 37° C. for 6 h. Next, organoids were washed twice with DMEM/F12 GlutaMAX medium (Thermo Fisher Scientific, Cat. No. 10565018) and plated in BME. Complete DMEM/F12 GlutaMAX medium (Thermo Fisher Scientific, Cat. No. 10565018), supplemented with 10 mmol/L Hepes (Thermo Fisher Scientific, Cat. No. 15630106), streptomycin/penicillin (1%, Thermo Fisher Scientific, Cat. No. 15140122), 10.08 ng/mL FGF (Thermo Fisher Scientific, Cat. No. PHG0261), B27 supplement (Thermo Fisher Scientific, Cat. No. 17504001) and 10 μmol/L Y-27632 (Bio Connect, Cat. No. S1049) was added to the organoids for 2 days. Organoids were selected with 0.5 μg/mL hygromycin (Thermo Fisher Scientific Cat. No. 10687-010).

### Isolation of Extracellular Vesicles by Ultracentrifugation

T cells isolated from the mouse spleen or Jurkat T cells were treated with PBS or with docetaxel (10 nmol/L and 5 nmol/L, respectively) for 72 h. Subsequently, conditioned media were collected, and T cells were removed by centrifugation at 1500 g for 5 min. Next, conditioned media were spun twice at 500 g for 10 min and twice at 2000 g for 15 min, at 40C. Subsequently, the EVs were isolated as previously described 37. Supernatant was centrifuged twice at 16,500 g for 20 min. The supernatant was next spun at 100,000 g for 1 h. Afterwards, the supernatant (containing soluble factors without the EVs) was removed and concentrated using Amicon Ultra columns with a 10 kDa cut off (Merck, Cat. No. UFC201024). These samples are the ‘EV-depleted fraction’. The pellets obtained after the first centrifugation at 100,000 g were pooled and centrifuged once more at 100,000 g for 1 h. The resulting pellets containing the EV-enriched fractions were resuspended in PBS and concentrated using Amicon Ultra 2 ml columns with a 10 kDa cut off (Merck, Cat. No. UFC201024). The EV-enriched and EV-depleted fractions were kept at −80° C. until further use. Each sample was resuspended in a specific volume to normalize the sample concentration to the initial number of T cells from which they are derived.

### T Cell Immunofluorescence Staining and Analysis

Mouse T cells isolated from the spleen and treated with a vehicle control or with docetaxel (10 nmol/L for 72 h) were seeded on glass coverslips pre-coated with poly-D-lysine (Thermo Fisher Cat. No. A3890401) and with αCD3 (eBioscience, Cat. No. 56-0033-80) for 15 min. Next, for immunofluorescence staining of tubulin, cells were extracted for 1 min with pre-warmed (37° C.) extraction buffer composed of MRB80 (80 mmol/L K-PIPES pH 6.8, 4 mmol/L MgCl2, 1 mmol/L EGTA) supplemented with 0.35% Triton X-100 and 0.2% glutaraldehyde, followed by fixation using pre-warmed (37° C.) 4% paraformaldehyde (PFA) in PBS for 10 min. Cells were washed with PBS and permeabilized with 0.2% Triton X-100 in PBS for 5 min. Epitope blocking and antibody labeling steps were performed in PBS with 3% BSA. After staining and washing with PBS, cells were air dried and mounted in DAPI-containing Vectashield mounting medium (Vector Laboratories, Cat. No. H-1200-10). The following primary antibody was used: rabbit anti-tubulin (clone EP1332Y, Abcam, Cat. No. ab52866). Secondary antibody was highly cross-adsorbed Alexa Fluor-594-conjugated goat antibody against rabbit IgG (Thermo Fisher Scientific). For labeling of actin, Alexa Fluor-488-conjugated Phalloidin (Thermo Fisher Scientific, Cat. No. 12379) was included during secondary antibody incubation.

For fluorescence intensity quantifications, samples were imaged on a spinning disk confocal setup consisting of an inverted research microscope Nikon Eclipse Ti-E (Nikon) equipped with the perfect focus system (Nikon), Plan Apo VC 100×N.A. 1.40 oil objective (Nikon), spinning disk Yokogawa CSU-X1-A1 with 405-491-561-642 quad-band mirror (Yokogawa). The system was equipped with an ASI motorized stage with piezo plate MS-2000-XYZ (ASI), Prime BSI sCMOS camera (Photometrics) and controlled by the MetaMorph 7.10 software. For excitation, 405 nm 100 mW Stradus (Voltran), 491 nm 100 mW Calypso (Cobolt), and 561 nm 100 mW Jive (Cobolt) lasers were used in combination with ET-BFP2 (49021, Chroma), ET-GFP (49002, Chroma), and ET-mCherry (49008, Chroma) filter sets, respectively. Z-stacks of cells were acquired with a step size of 0.25 μm.

All images were processed using ImageJ. For quantification of fluorescence intensity, sum projections were made for each z-stack using ImageJ. Next, the raw integrated density of a square box around individual cells of interest per image, as well as an empty region in the field of view, were calculated using ImageJ. Per image, the background intensity from the empty region was subtracted from the measured integrated density for each cell in the field of view to give the background corrected intensity per cell.

Having now fully described this invention, it will be appreciated by those skilled in the art that the same can be performed within a wide range of equivalent parameters, concentrations, and conditions without departing from the spirit and scope of the invention and without undue experimentation.

Reference to known method steps, conventional methods steps, known methods or conventional methods is not in any way an admission that any aspect, description, or embodiment of the present invention is disclosed, taught, or suggested in the relevant art.

### Example 3

### Results

### T Cells Mediate the In Vivo Anti-Tumor Effects of Taxanes.

Considering that taxanes-induced cell death is classically thought to be mediated by impairing mitotic progression, it was first tested whether this mechanism held true in vivo in two immunocompetent mouse models of breast cancer that are based on the loss of tumor suppressor genes Brca1 and Trp53 (KB1P model) or on the overexpression of the oncogene PyMT (MMTV-PyMT model).

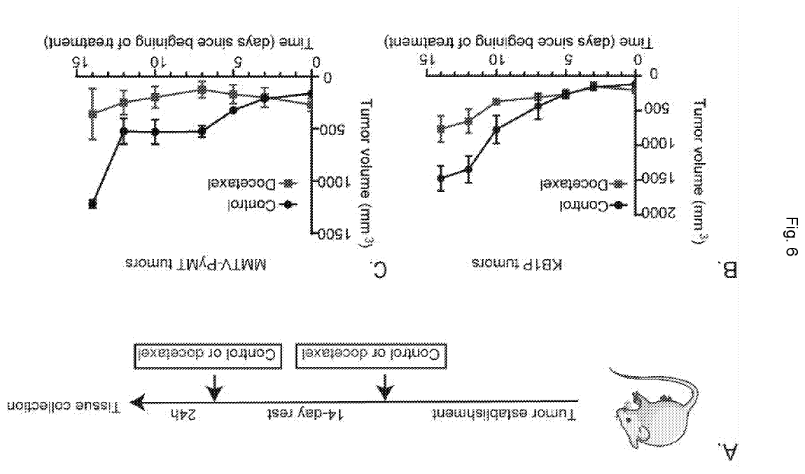

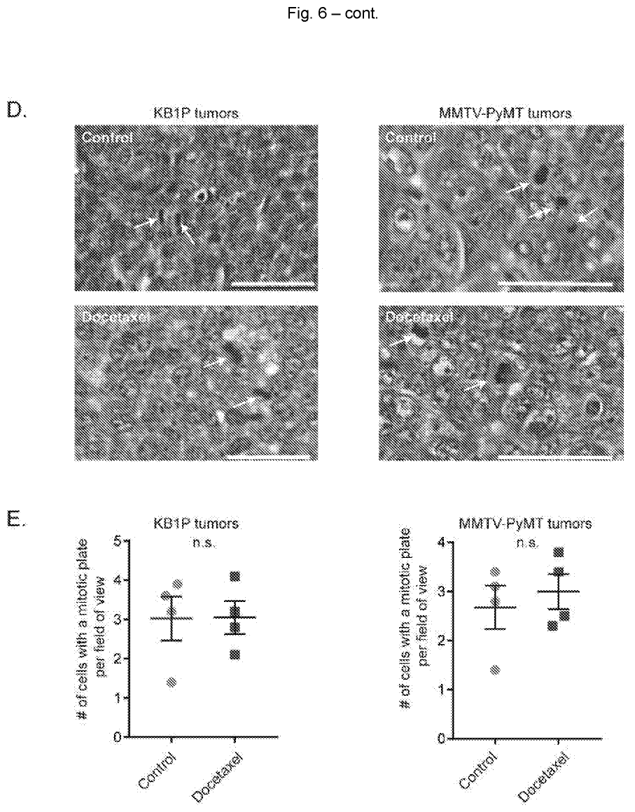

Docetaxel treatment in mice bearing tumors delayed tumor outgrowth, confirming that these tumor models were sensitive to docetaxel (FIG. 6A-C). However, we did not observe an accumulation of mitotic figures in tumors isolated 24 h after the second docetaxel administration (FIG. 6D, E), which would be expected if the anti-tumor effects of docetaxel occurred through perturbation of mitosis. This suggested that killing of cancer cells upon docetaxel in KB1P and MMTV-PyMT tumors was not mediated by delayed mitotic progression in vivo.

This prompted to investigate the differing mode of action of docetaxel in the in vivo setting. For this we interrogated the biological relevance of this correlation in mouse models of breast cancer. Friend Virus B (FVB) mice carrying KB1P tumors were subjected to treatment with an IgG control or with depleting antibodies against CD4 and CD8 expressing cells, as previously achieved (Ritsma, L., N. Vrisekoop, and J. van Rheenen, Nat Commun, 2013. 4: p. 2366), prior to treatment with either AC or AC+T (FIG. 1A). The reduction of intratumoral T cells upon treatment with depleting antibodies was confirmed by immunohistochemistry (FIG. 7).

In both the IgG control mice and the T cell-depleted mice, treatment with AC led to a partial response with a persistent tumor during the course of the treatment (dotted line in FIG. 1B). Importantly, AC+T triggered a complete response with no detectable tumors during the course of the treatment in the IgG control but not in the T cell-depleted mice (FIG. 1C). This data suggest that T cells collaborate with AC+T in controlling tumors in vivo.

To further test whether T cells mediated the anti-tumor activity of docetaxel, we used Fluorescence-Activated Cell Sorting (FACS) to isolate T cells from KB1P mammary tumors grown in mice that had been treated with two cycles of either control, AC or AC+T (FIG. 1D and FIG. 8A). FACS quantification revealed an increased infiltration of T cells into tumors treated with AC+T compared to control or AC-treated tumors (FIG. 8B).

Subsequently, the cytotoxic capacity of the isolated intratumoral T cells was tested by co-culturing them with matched KB1P tumor-derived organoids (FIG. 1D). Co-cultures with AC+T-treated intratumoral T cells led to a reduction in the number of surviving organoids and an increase in organoid apoptosis compared to the co-cultures with control or AC-treated intratumoral T cells (FIG. 1E,F).

Next, we tested whether these observations held true for MMTV-PyMT tumors. Similar to KB1P tumors, we observed a higher infiltration of T cells upon AC+T treatment (FIG. 8C). Additionally, co-cultures with T cells isolated from AC+T-treated tumors led to reduced organoid viability and enhanced organoid apoptosis in comparison to their co-culture counterparts with T cells isolated from control or AC-treated MMTV-PyMT tumors (FIG. 8D).

We next investigated the individual roles of CD4 versus CD8 T cells in driving tumor response to docetaxel (FIG. 1G). In line with the increased intratumoral T cell infiltration upon AC+T (FIG. 8B), docetaxel treatment correlated with enhanced CD4 and CD8 T cell infiltration in KB1P tumors (FIG. 8E, F).

In addition, depletion of either CD4 T cells alone, CD8 T cells alone or both CD4 and CD8 T cells abrogated the anti-tumor effects of docetaxel treatment in both KB1P and MMTV-PyMT tumors (FIG. 1H and FIG. 8G). This demonstrated that both CD4 and CD8 T cell populations contributed to tumor response to docetaxel treatment.

### Docetaxel Directly Increases T Cell Cytotoxic Activity.

We next investigated how docetaxel triggered the observed enhanced cytotoxic capacity of T cells. We hypothesized that docetaxel might either alter the immune properties of tumor cells or influence the characteristics of the T cells. Considering that untreated tumor organoids were more efficiently killed by T cells isolated from tumors treated with AC+T compared to AC alone (FIG. 1E, F), the enhanced T cell cytotoxicity might not be caused by modulating the immune properties of the cancer compartment. To confirm this, we tested the ability of T cells isolated from control KB1P tumors to kill KB1P organoids that were pre-treated with low doses of AC or AC+T (FIG. 9A). After 72 h of pre-treating the organoids, we renewed the culture media and co-cultured these organoids with T cells isolated from control-treated KB1P tumors (FIG. 9A). We found that the addition of docetaxel (AC+T) did not alter the number of living organoids upon co-culture with those T cells (FIG. 9B). This confirmed that the anti-tumor activity of docetaxel was not caused by enhanced sensitivity of cancer cells to T cell-mediated killing.

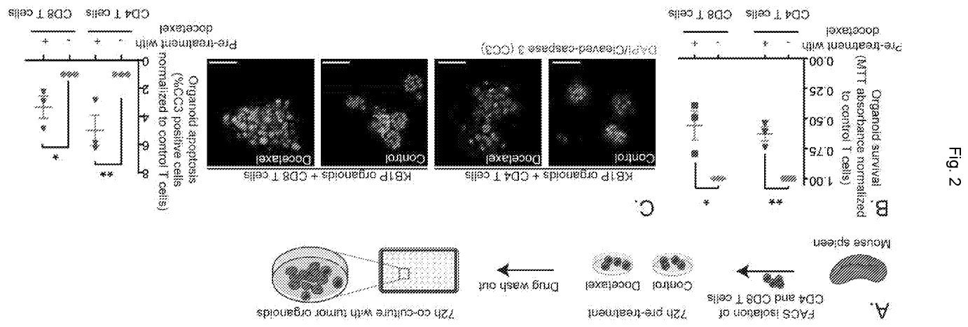

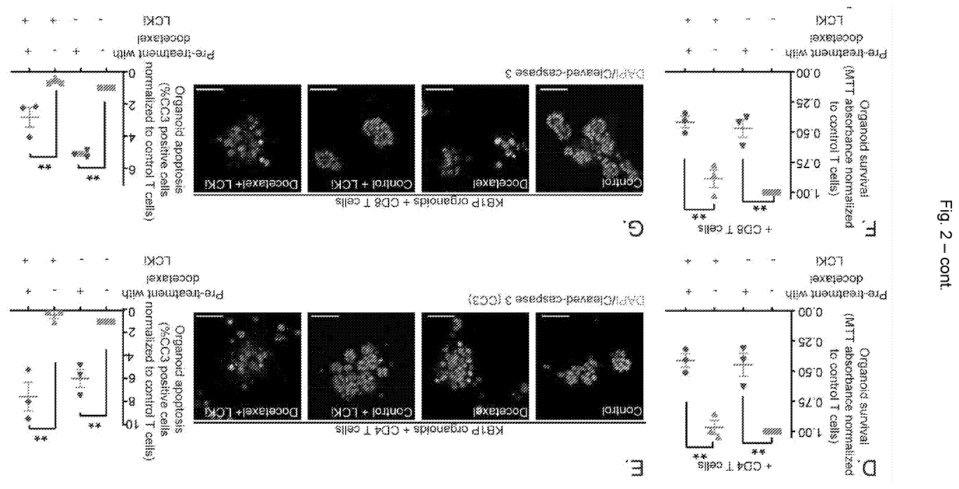

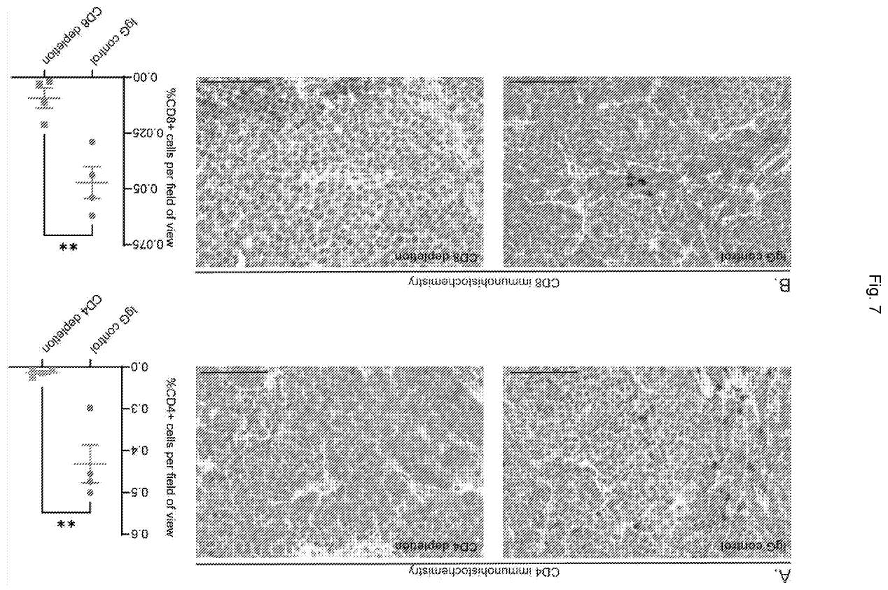

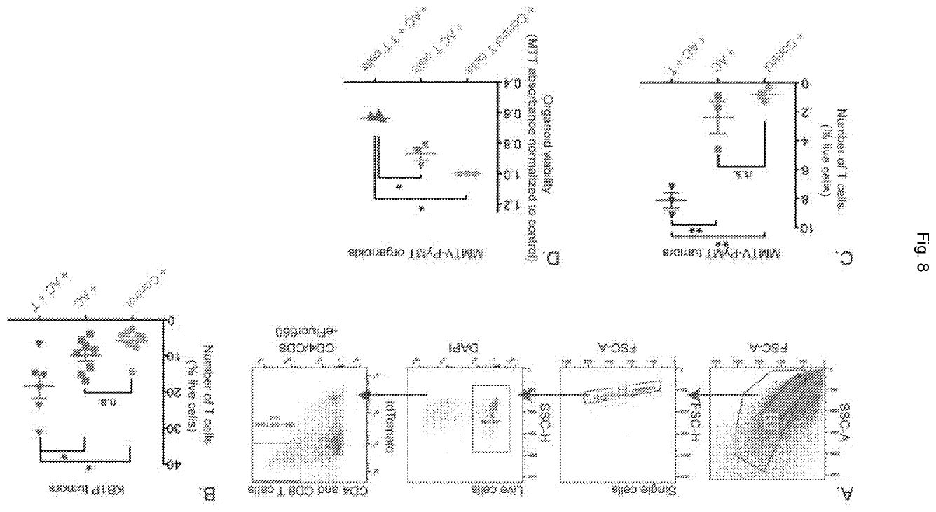

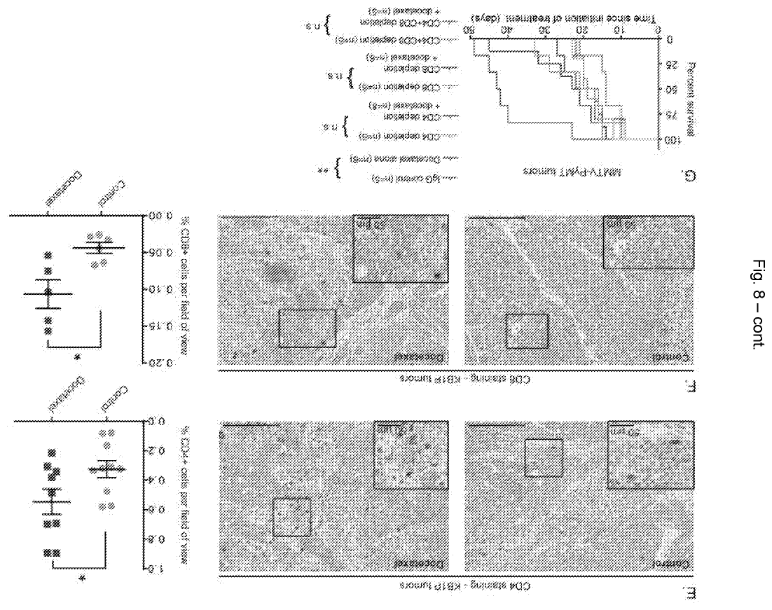

We then assessed whether docetaxel might instead directly influence the killing ability of T cells. To test this, CD4 and CD8 T cells were separately isolated by FACS from the spleens of non-tumor bearing, drug naïve FVB female mice (FIG. 2A and FIG. 10A). Following isolation, splenic T cells were treated in vitro for 72 h with 10 nmol/L of docetaxel, a dose that did not induce significant killing of T cells but that increased microtubule density, in line with the known microtubule-stabilizing effect of taxanes, and that is relevant to the dose used in vivo (FIG. 2A and FIG. 10B, and FIG. 10C), prior to co-culturing these T cells with drug-naïve KB1P organoids. Intriguingly, we observed a reduction of living organoids and an increased apoptotic rate in organoids co-cultured with either CD4 or CD8 T cells pre-treated with docetaxel compared to their control counterparts (FIG. 2B, C).

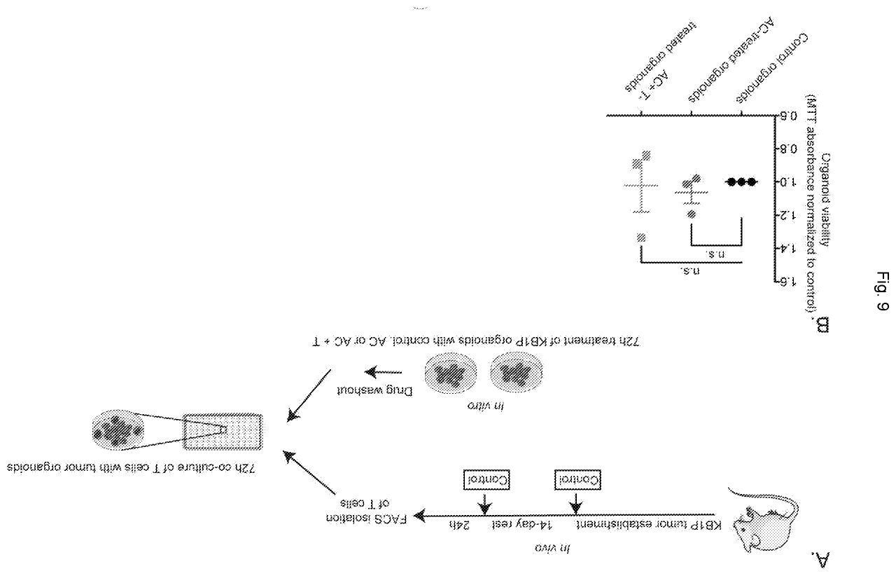

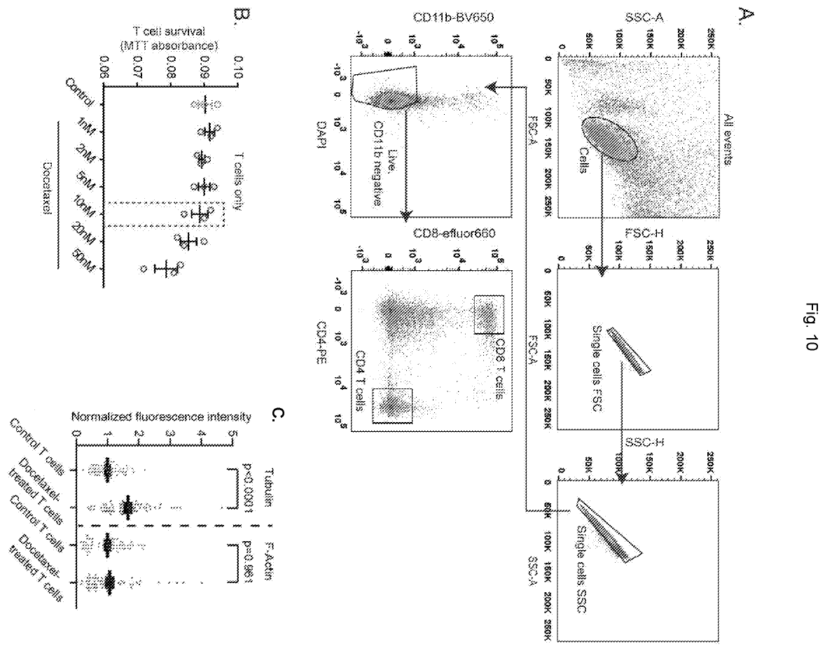

To confirm the taxane-specific induction of T cell killing capacity, splenic T cells were treated with a low dose of paclitaxel (another taxane) or carboplatin (a non-taxane chemotherapy, FIG. 11A, B) prior to co-culturing them with tumor-derived organoids from the KB1P and the MMTV-PyMT models. Similar to our previous observations, paclitaxel-treated T cells triggered a decrease in tumor organoid survival compared to control T cells, whereas carboplatin-treated T cells did not (FIG. 11C-F), thereby confirming the taxane-specificity of our observations.

Classical T cell-mediated killing requires specific recognition of an antigen presented by MHC molecules on the target cell by the T cell receptor (TCR). However, because we isolated T cells from spleens of tumor-naïve mice, which were co-cultured with tumor organoids for only a short duration, this specific tumor organoid-T cell interaction was lacking in our setup (FIG. 2A). Therefore, we hypothesized that elimination of tumor organoids by T cells pre-treated with docetaxel was independent of a specific TCR-antigen recognition.

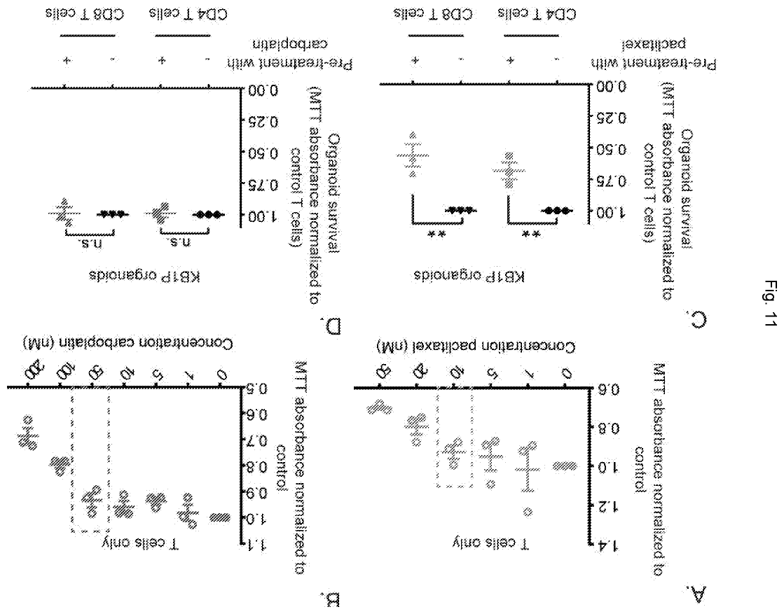

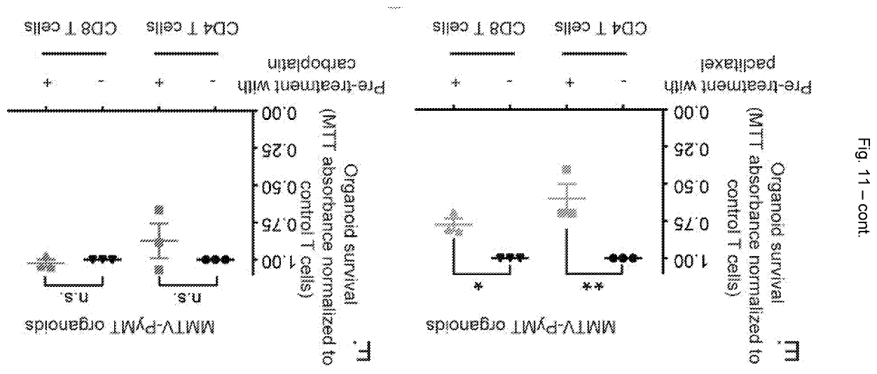

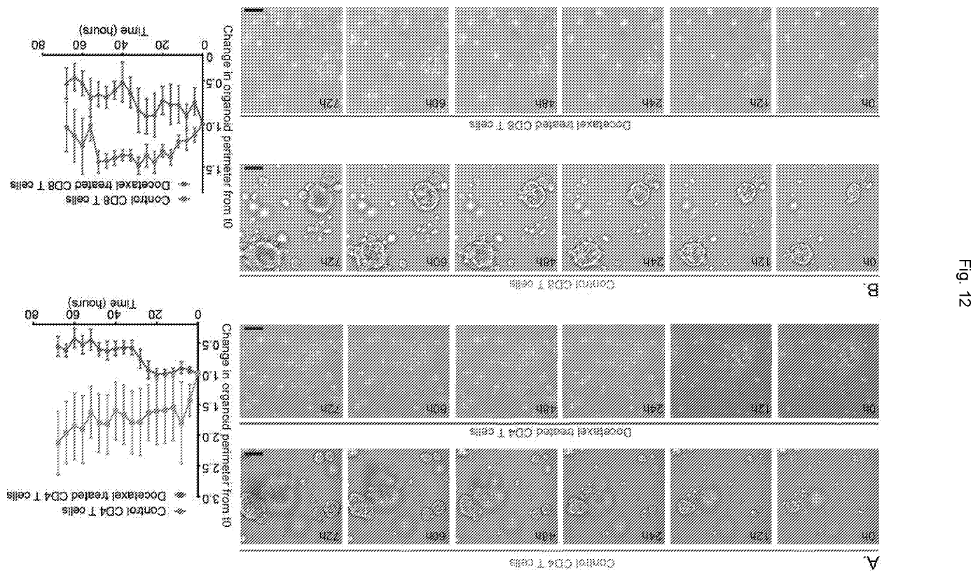

To test this, we isolated splenic T cells from Rosa26mTmG FVB mice and employed live microscopy to visualize their interactions with KB1P organoids over time in vitro. In line with our T cell-mediated killing assay (FIG. 2B, C), we observed a size reduction of KB1P organoids co-cultured with either CD4 or CD8 T cells over time (FIG. 12A, B). This killing of cancer organoids was not accompanied by infiltration of T cells into the basement membrane extract (BME) to reach the organoids (FIG. 12C, D). This demonstrated that T cell-mediated killing upon docetaxel treatment did not require physical contact between a T cell and a cancer cell.

To confirm this, we assessed whether inhibition of TCR signaling by using a potent and selective inhibitor of Lymphocyte Cell Kinase (LCKi) (Burchat, A. F., et al. Bioorg Med Chem Lett, 2000. 10(19): p. 2171-4) would influence the T cell-killing capacity upon docetaxel treatment. LCK inhibition reduced killing of MMTV-PyMT organoids expressing OVA by T cells isolated from inbred OT I mice (Hogquist, K. A., et al., 1994, T cell receptor antagonist peptides induce positive selection. Cell 76, 17-27. 10.1016/0092-8674(94)90169-4) (Clarke, S. R. et al., 2000, Characterization of the ovalbumin-specific TCR transgenic line OT-I: MHC elements for positive and negative selection. Immunol Cell Biol 78, 110-117. 10.1046/j.1440-1711.2000.00889.x), confirming the ability of the LCK inhibitor to block TCR-mediated killing (FIG. 2L). LCK inhibition did not impair the ability of docetaxel to increase the cytotoxic activity of either CD4 or CD8 T cells against KB1P organoids (FIG. 2D-G). The direct and TCR-independent increase of T cell-mediated killing upon docetaxel treatment was also confirmed in MMTV-PyMT organoids (FIG. 2H-K).

Lastly, to genetically confirm our findings, we isolated T cells from the spleen of NOD-SCID IL2rg-/- mice, which cannot mature nor produce a TCR response. Pre-treatment with docetaxel triggered T cell-mediated killing of KB1P organoids in this setting (FIG. 12E), further confirming the TCR-independent killing by docetaxel-treated T cells. Together, our data provide evidence that docetaxel treatment induces T cells to kill cancer cells independent of TCR activation.

### Docetaxel Increases the Anti-Tumor Activity of Human T Cells.

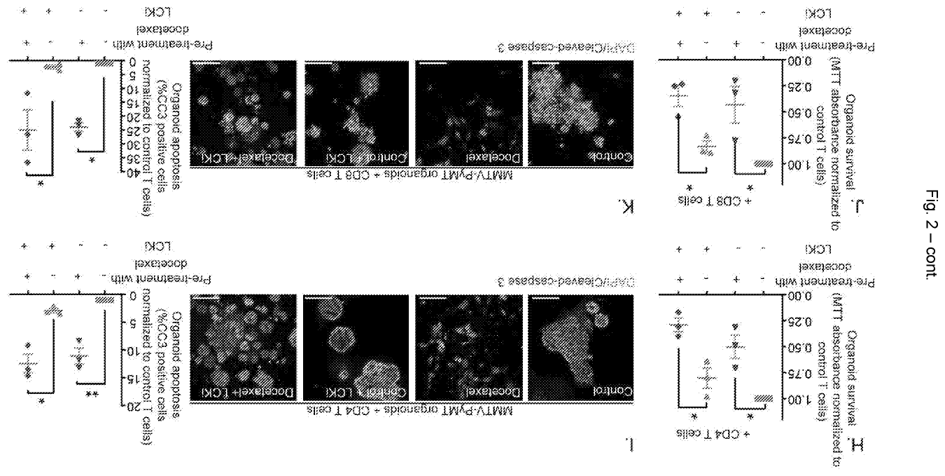

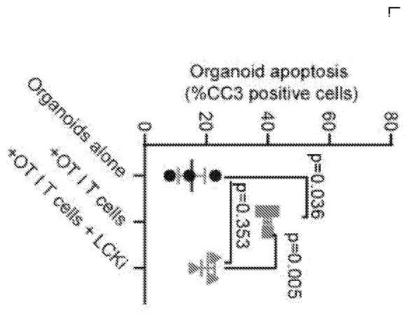

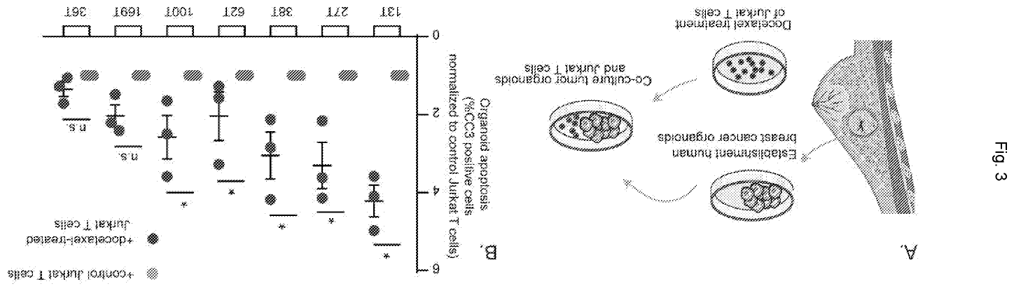

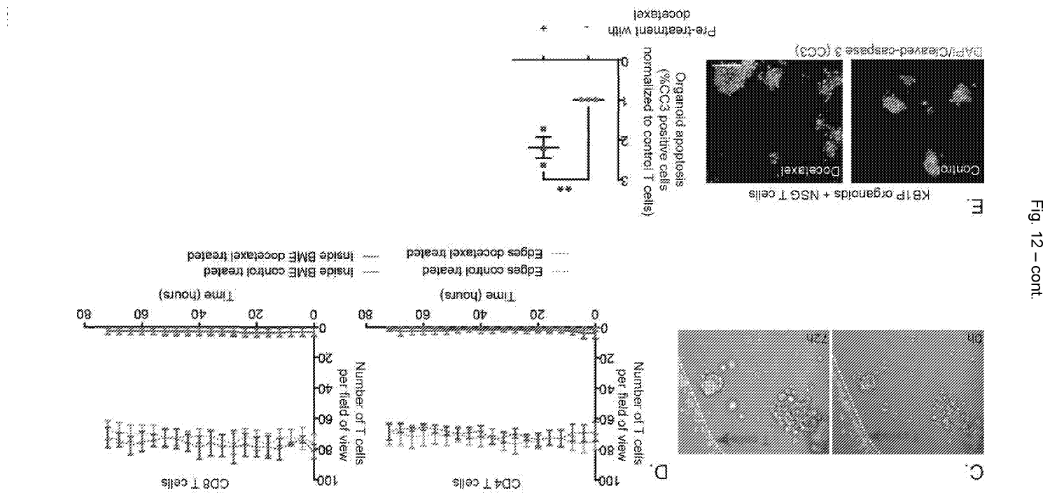

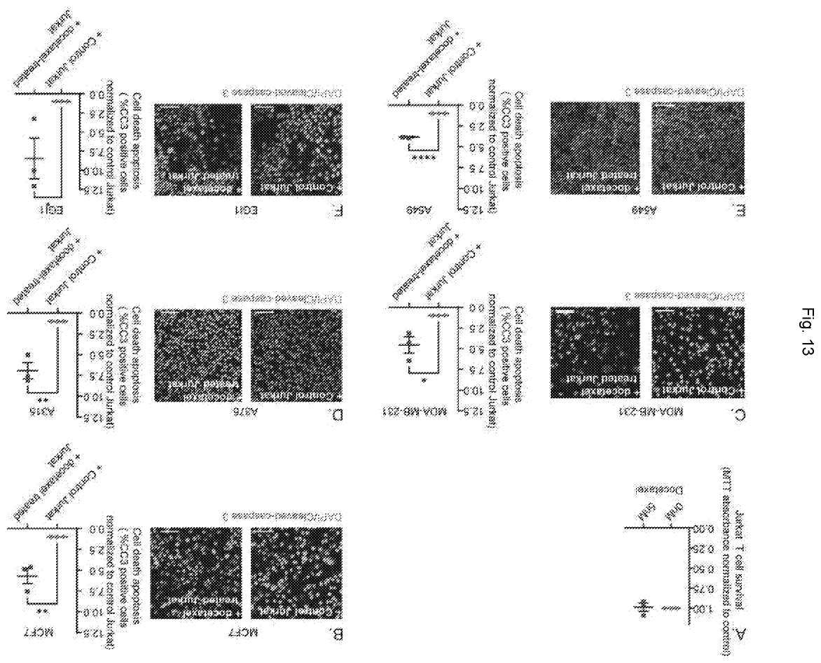

We next tested the ability of docetaxel to induce T cell-mediated killing in human cancer material. First, we worked with Jurkat T cells, an immortalized line of human T lymphocyte cells that is frequently used to study T cell signaling. Jurkat T cells were pre-treated with 5 nmol/L of docetaxel, a dose that did not significantly reduce Jurkat T cells survival (FIG. 13A), prior to being co-cultured with 7 breast cancer patient-derived organoid lines (FIG. 3A, Dekkers, J. F., et al. Nat Protoc, 2021. 16(4): p. 1936-1965). Similarly, to the mouse setting described above (FIG. 2), there was no specific TCR-antigen recognition between Jurkat cells and the breast cancer patient-derived organoids. Analysis of cleaved-caspase 3 immunofluorescence staining revealed that in 5 out of the 7 patient-derived organoid lines tested, docetaxel-treated Jurkat T cells significantly enhanced cancer organoid apoptosis compared to control Jurkat T cells (FIG. 3B, C).

We also tested the ability of docetaxel to induce Jurkat T cell-mediated killing in cells derived from human breast tumors, lung adenocarcinoma, melanoma, glioma, cholangiocarcinoma, hepatocellular carcinoma, or rectal carcinoma (FIG. 13B-J). In this context, we found that Jurkat T cells pre-treated with docetaxel significantly increased cancer cell apoptosis upon co-culture compared to control Jurkat T cells (FIG. 13B-J).

We next interrogated whether docetaxel could not only activate cytotoxic responses in immature T cells, but also enhance the cytotoxic activity of T cells that are specifically reactive to cancer cells. We previously generated a platform to induce tumor-reactive patient-derived T cells and to study their interactions with matched patient-derived tumor organoids (Cattaneo, C. M., et al., Nat Protoc, 2020 15(1): p. 15-39; Dijkstra, K. K., et al. Cell, 2018. 174(6): p. 1586-1598 e12). We employed a previously validated pair of tumor-reactive T cells and matched tumor organoids isolated from a lung cancer patient to test whether docetaxel treatment of T cells could further increase their anti-tumor potential (Dijkstra, K. K., et al. Cell, 2018. 174(6): p. 1586-1598 e12).

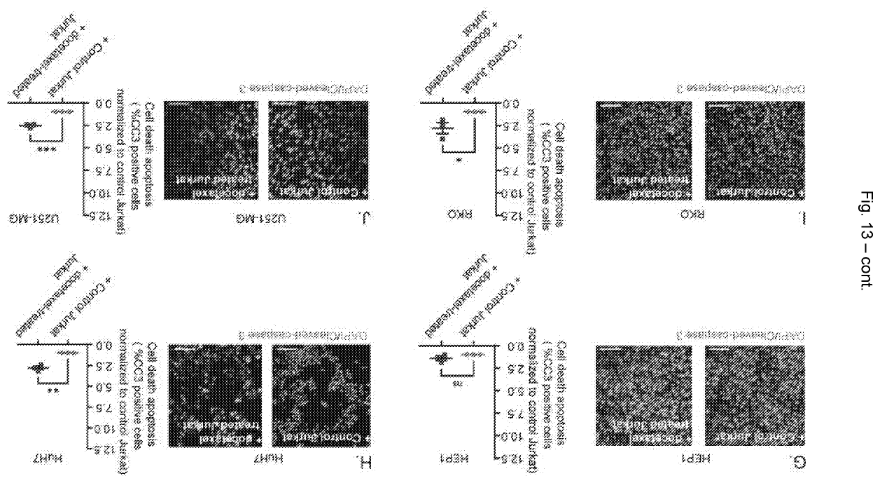

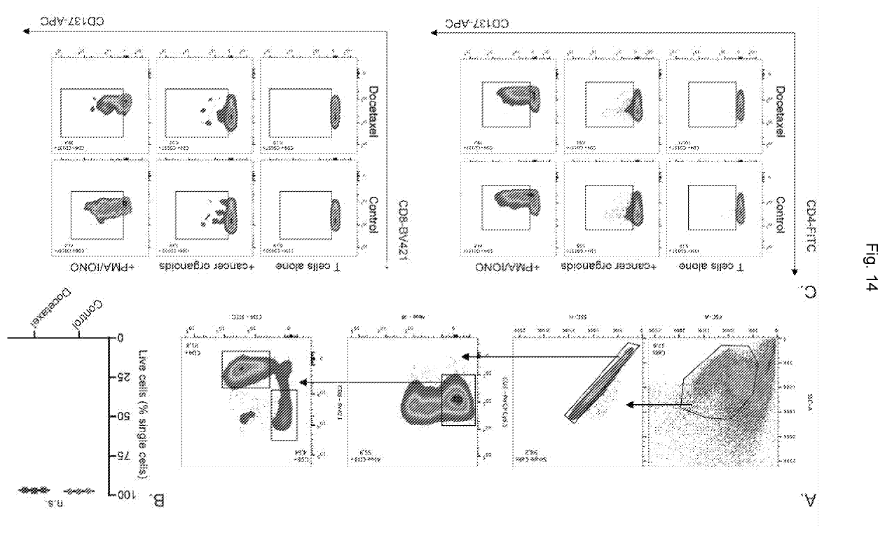

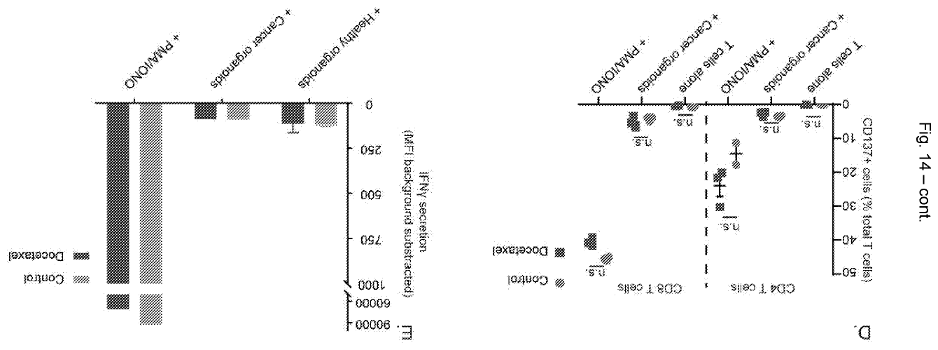

Tumor-reactive T cells were induced by culturing peripheral blood mononuclear cells (PBMCs) with autologous tumor organoids, as previously described (Cattaneo, C. M., et al., Nat Protoc, 2020 15(1): p. 15-39; Dijkstra, K. K., et al. Cell, 2018. 174(6): p. 1586-1598 e12). Subsequently, T cells were pre-treated for 72 h with 10 nmol/L of docetaxel (FIG. 3D), a dose that did not significantly alter T cell viability (FIG. 14A, B). In addition, docetaxel did not increase the expression of CD137, CD107 or PD1 (FIG. 14D, F-G).

In addition, assessment of CD137, a marker of tumor-reactive T cells, demonstrated that docetaxel did not increase the ability of T cells to recognize tumor organoids (FIG. 14C, D). In line with this, docetaxel did not enhance IFNγ secretion by T cells (FIG. 14E), confirming that docetaxel does not increase the ability of T cells to recognize tumor organoids and does not induce canonical T cell activation in this setting, in line with our findings in mouse T cells. Next, we co-cultured tumor-reactive T cells with matched autologous cancer organoids (FIG. 3D) and healthy organoids. Assessment of organoid viability following co-culture demonstrated that pre-treating T cells with docetaxel indeed increased their anti-tumor capacity against tumor organoids (FIG. 3E,F) but not against healthy organoids.

Together, our data demonstrate that docetaxel directly increased the ability of T cells to eliminate tumor cells, both in mouse naïve T cells, human naïve T cells and human tumor-reactive T cells. Importantly, docetaxel-mediated activation of T cell-mediated killing did not display hallmarks of classical T cell activation.

This prompted us to next dissect this alternative, novel mechanism of T cell activation driven by docetaxel.

### Docetaxel Induces Release of Cytotoxic Extracellular Vesicles by T Cells to Eliminate Tumor Organoids.

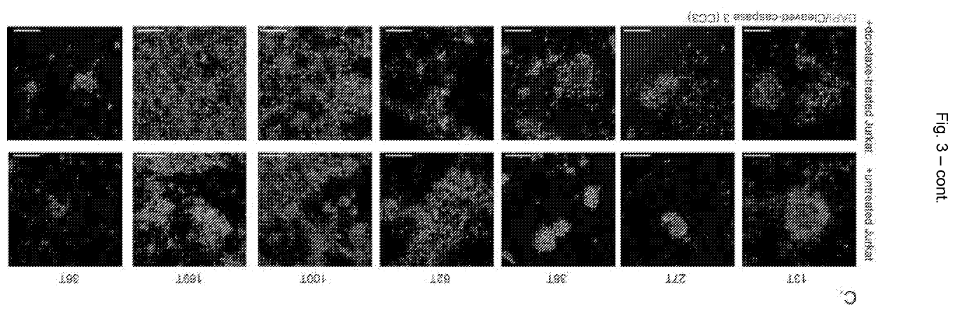

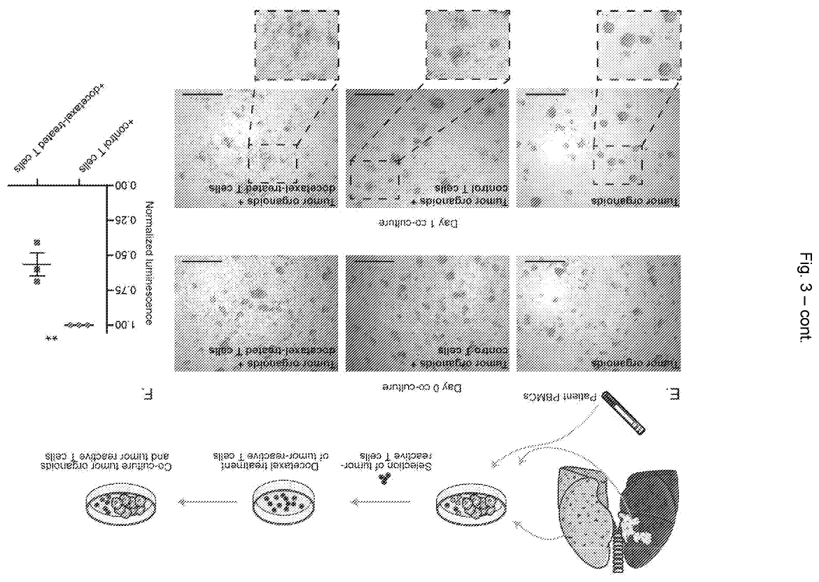

Our data demonstrated that T cell-mediated killing upon docetaxel did not require a direct contact between T cells and tumor organoids (FIG. 12C, D) and was not mediated by the TCR (FIG. 2D-K). This prompted us to assess whether docetaxel promoted T cells to release non-classical cytotoxic factors, which in turn eliminated tumor organoids. We first tested the response of KB1P and MMTV-PyMT organoids to treatment with conditioned media (CM) generated by T cells pre-treated with control or docetaxel. We found that both cancer organoid lines had a reduced live organoid count and increased organoid apoptosis when treated with CM derived from either CD4 or CD8 T cells that were pre-treated with docetaxel compared to the control pre-treated T cell cultures (FIG. 4A-D).

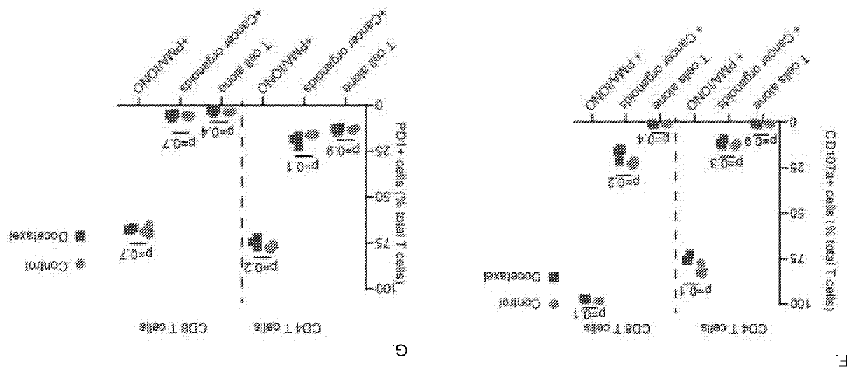

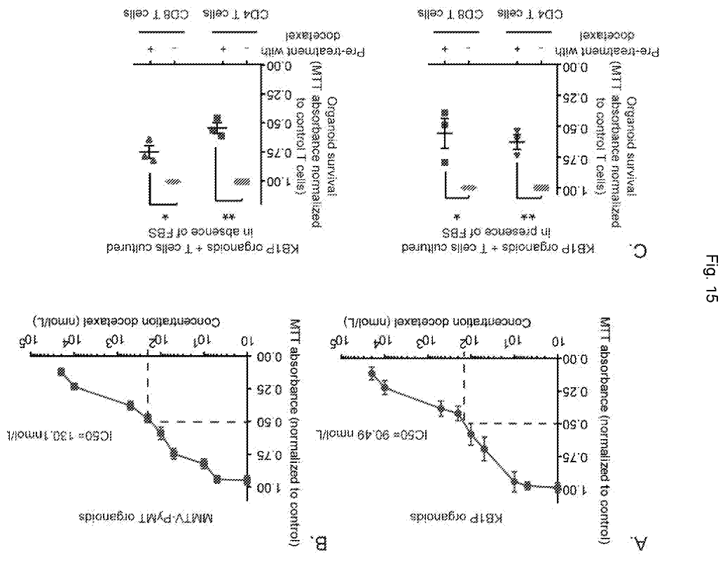

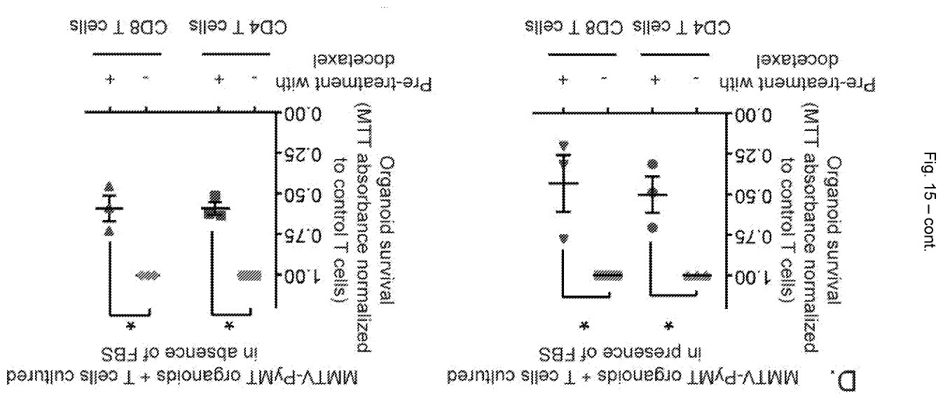

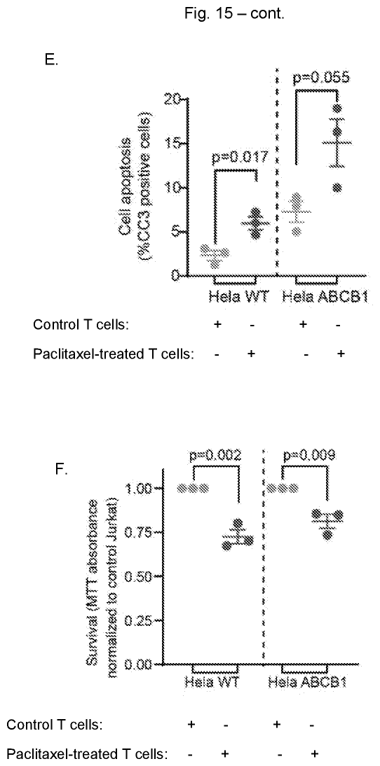

Importantly, we measured the sensitivity of KB1P and MMTV-PyMT cancer organoids to docetaxel and found that the IC50 of both lines (IC50(KB1P)=90.49 nmol/L and IC50(MMTV-PyMT)=130.1 nmol/L) was much higher than the concentration of docetaxel present in CM (10 nmol/L before diluted with organoid medium 1:1) (FIG. 15A, B). This confirmed that the reduced cancer organoid survival and the induction of organoid apoptosis upon CM treatment was caused by factors released by T cells upon docetaxel treatment rather than by docetaxel that was still present in the CM. Moreover, to test whether taxane is stored by T cells during treatment and released to target cells, we co-cultured paclitaxel-treated Jurkat T cells with Hela cells that overexpressed ABCB1 (Pgp). Pgp is an ABC transporter that drives the efflux of compounds such as paclitaxel, and thereby provides resistance to this drug (Data not shown). Paclitaxel-treated Jurkat T cells induced apoptosis and reduced survival in Hela ABCB1 cells to a similar level as for Hela WT cells (FIG. 15E, F). It is therefore unlikely that cancer cell killing by taxane-treated T cells is caused by an uptake and release of taxanes by T cells.

In order to identify the secreted factors that may be potentially responsible for killing tumor organoids, we next used mass-spectrometry to profile the secretome of CD4 and CD8 T cells treated with docetaxel. Because fetal bovine serum (FBS) used in culturing media contains many proteins that could interfere with mass-spectrometry analysis, we first confirmed that T cells pre-treated with docetaxel could also target KB1P and MMTV-PyMT organoids in the absence of FBS (FIG. 15C, D). We grouped CD4 and CD8 T cells together in order to obtain enough secreted protein for analysis.

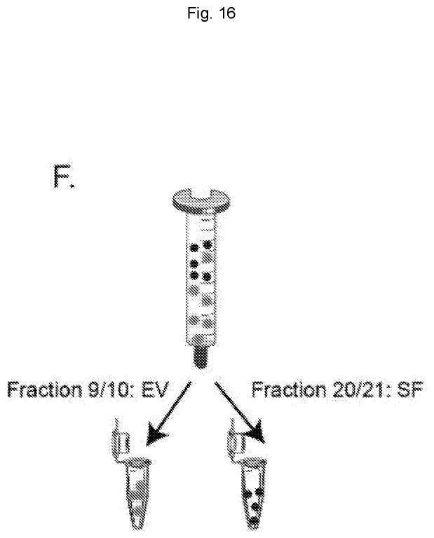

Intriguingly, the number of proteins in the CM from docetaxel-treated T cells was much higher compared to that from control T cells (FIG. 4E). We did not find significant differences in factors that have been reported to drive canonical T cell-mediated killing. Instead, pathway analysis revealed that peptides that are known to be the cargos of extracellular vesicles (EVs) were significantly more abundant in the CM from docetaxel-treated T cells (FIG. 4E). This led us to isolate EVs from the conditioned media generated by splenic T cells that had been treated with control or docetaxel through size exclusion chromatography (FIG. 16).

Electron microscopy and NanoSight particle analysis were employed to study the properties of the isolated EVs (FIG. 4F, G). In the fractions isolated from docetaxel-treated and control groups, we did not observe differences in EV density or size (FIG. 4F, G).

In line with our data in the CM (FIG. 4E), proteomics analysis of the EV fractions showed an increased number of proteins in the EV fraction isolated from docetaxel-treated T cells compared to the EV fraction from the control T cells (FIG. 4L), which indicates either a differential abundance of EVs or of cargo proteins in EVs upon docetaxel treatment of the T cells.

KB1P and MMTV-PyMT organoids were subsequently treated with these EV fractions (isolated from 6×10\hat{ }6 million T cells). (Please note that in the experiments the EV fraction obtained from the same amount of T cells were used) Immunofluorescence analysis of cleaved-caspase 3 demonstrated that EVs released by T cells treated with docetaxel induced apoptosis to a significantly larger extent than the EVs released by control T cells (FIG. 4H, I). Importantly, tumor organoids were also treated with the soluble fraction (SF) isolated from the CM of T cells that contained secreted soluble proteins but no EVs. We did not observe an induction of apoptosis in either organoid lines (FIG. 4J, K). Together, these experiments suggest that docetaxel induces T cells to release cytotoxic factors, and that the EV fraction is mediating this killing.

Additionally, we separated the CM into an EV-enriched and an EV-depleted fractions using a second method that involves serial ultracentrifugation steps, as previously described (Zomer, A., et al. (2015). In Vivo imaging reveals extracellular vesicle-mediated phenocopying of metastatic behavior. Cell 161, 1046-1057. 10.1016/j.cell.2015.04.042.). In this context, we again observed an induction of apoptosis in KB1P and MMTV-PyMT organoids treated with the EV-enriched fraction isolated from docetaxel-treated T cells compared to control T cells, while treatment with the EV-depleted fraction did not (Data not shown). Lastly, we isolated the EV-enriched fraction from 118×10\hat{ }9 docetaxel-treated human Jurkat T cells, and also confirmed the dose-dependent killing capacity of the EV-enriched fraction in human MDA-MB-231 tumor cells (FIG. 4M). Interestingly, the amount of EVs required to induce similar levels of apoptosis appeared to be higher in the 2D MDA-MB-231 cell line compared to the mouse 3D tumor organoids (FIG. 4M versus FIG. 4H, I). This shows that all kind of cultures are sensitive to EV from taxane-treated T cells. The dose adjustment is a matter of routine by the skilled person in the art.

We next asked whether patient-derived tumor-reactive T cells also release EVs with killing capacity upon treatment with docetaxel. We treated tumor-reactive T cells isolated from two NSCLC patients (patient 1 and patient 2) with control or docetaxel and we isolated the EV fraction and soluble fraction from the T cell CM. Subsequently, we exposed autologous tumor and healthy organoids to those fractions. Moreover, we treated tumor and healthy organoids isolated from a colorectal patient (patient 3, for which we could not expand enough tumor-reactive T cells) with the EV fraction and soluble fraction of the tumor-reactive T cells of patient 2. In line with our observations in mouse T cells (FIG. 4H, I), we found in all three patient-derived material that EVs generated by tumor reactive T cells treated with docetaxel triggered apoptosis in tumor organoids, while the soluble fraction did not (FIG. 3G-I). Moreover, apoptosis was not increased in healthy organoids upon treatment with the EV fraction or the soluble fraction (FIG. 3J, K). Collectively, we find that docetaxel increases tumor-specific T cell killing in cancer patient material by inducing a release of cytotoxic EVs.

### T Cells Pre-Treated With Docetaxel Ex Vivo Can Eliminate Tumors In Vivo.

Because docetaxel-treated T cells eliminated tumor organoids in vitro, it might be possible that injecting docetaxel-treated T cells into mice bearing tumors would reduce tumor growth. However, this approach would only be beneficial if T cell-mediated killing upon docetaxel is specific to cancer cells and does not lead to untargeted killing of all cells of the recipient mouse or patient. To test this, we evaluated the killing capacity of mouse splenic T cells pre-treated with docetaxel against healthy mouse mammary epithelial cells (MECs).

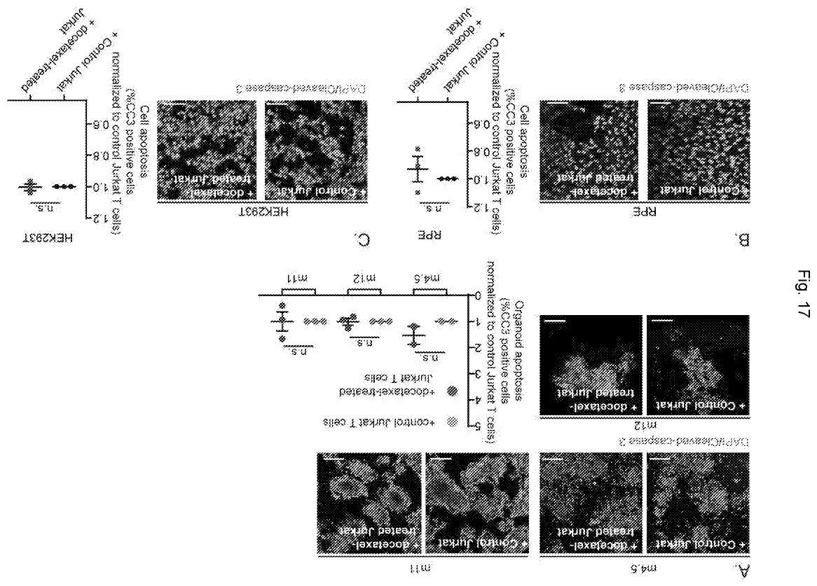

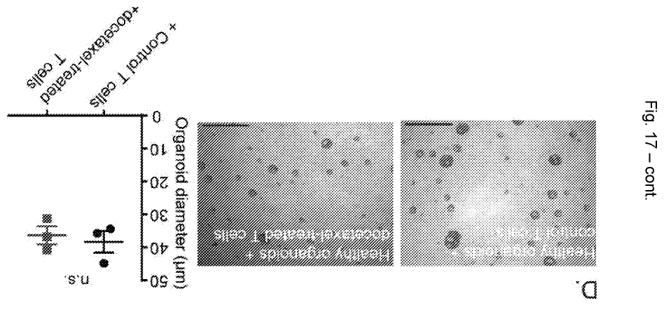

Intriguingly, we found that apoptosis was induced in only a minor fraction of MECs that were co-cultured with either CD4 or CD8 T cells pre-treated with docetaxel, in contrast to the high levels of cell death in cancer organoids (FIG. 5A). Additionally, MEC survival was not significantly different when co-cultured with control-or docetaxel-treated CD4 or CD8 T cells (FIG. 5B). Moreover, we co-cultured Jurkat T cells with 3 human milk-derived organoid lines (m4.5, m11 and m12 Dekkers, J. F., et al. Nat Protoc, 2021. 16(4): p. 1936-1965) and with retinal pigment epithelium (RPE) cells and human embryonic kidney 293 (HEK293T) cells, two human lines derived from healthy tissues. In all 5 human healthy lines, we did not observe an induction of apoptosis upon co-culture with docetaxel-treated Jurkat T cells compared to control Jurkat T cells (FIG. 17A-C).

Lastly, we tested the killing capacity of human tumor-reactive T cells pre-treated with docetaxel against human healthy lung organoids derived from the same lung cancer patient as before (FIG. 3D and Dijkstra, K. K., et al. Cell, 2018. 174(6): p. 1586-1598 e12). We did not observe killing of the healthy organoids when co-cultured with docetaxel-treated tumor reactive T cells (FIG. 17D). Overall, docetaxel-treated T cells (either mouse splenic T cells, Jurkat T cells or tumor-reactive human T cells) induced apoptosis in 15/18 cancer lines and 0/7 healthy lines (FIG. 5C). Together, these findings demonstrate that docetaxel triggers T cells to kill tumor organoids specifically, without significantly affecting the viability of healthy epithelial cells.

Considering that T cell-mediated killing upon docetaxel treatment is driven by EVs, we next hypothesized that the preferential killing of cancer organoids compared to healthy epithelial cells may be due to a differential capacity for uptake of EVs derived from T cells. We isolated EVs from mouse splenic T cells pre-treated with control or with docetaxel. We next incubated MECs, KB1P organoids and MMTV-PyMT organoids with EVs labelled with a fluorescent dye (PKH67). Quantification of EV-PKH67 uptake after 1 h of incubation did not reveal differential uptake of EVs derived from control treated T cells compared to docetaxel-treated T cells (FIG. 5D). However, we observed a significantly higher uptake of EVs in cancer organoids compared to MECs (FIG. 5D). Collectively, this suggests that healthy epithelial cells are protected from T cell-mediated killing upon treatment with docetaxel because of their reduced EV-uptake capacity. This prompted us to assess whether T cells pre-treated with docetaxel in vitro could specifically target tumors growing in vivo.

We isolated splenic T cells from healthy, non-tumor bearing mice and pre-treated them with docetaxel for 72 h as before (FIG. 2A). Subsequently, we intravenously injected a single dose of T cells into mice bearing KB1P or MMTV-PyMT tumors (FIG. 5E). Strikingly, in mice that received docetaxel-treated T cells, we observed significantly slower tumor growth compared to mice that were injected with control T cells (FIG. 5F, G). This demonstrated that T cells pre-treated with docetaxel ex vivo had the capacity to kill tumor cells growing in vivo and to halt tumor growth. This also suggested that the anti-tumor capacity of T cells pre-treated with docetaxel was maintained for over ten days in vivo.

Importantly, in both tumor models, a single transplantation of docetaxel-treated T cells resulted in a significant increase in mouse survival compared to the survival of mice that received control T cells (FIG. 5H, I). Together, this data illustrates that pre-treating T cells ex vivo with docetaxel may be used to control tumor growth in vivo while removing the cytotoxicity that accompanies systemic docetaxel treatment.

New therapies based on T cells including Tumor-infiltrating lymphocytes (TILs) or CAR-T cells are extensively developed for clinical use for cancer. However, these therapies are based on a TCR mediated cytotoxicity, whilst we here show that docetaxel induces a non-canonical and TCR independent T cell cytotoxicity. We also tested the ability of docetaxel to affect T cell killing capacity in the context of tumor-specific T cells. For this purpose, we used again the OT I/OVA system, in which the TCR of T cells are designed to recognize the OVA epitope expressed here by the MMTV-PyMT OVA+ organoids. As expected for TCR mediated cytotoxicity, control OT I T cells induced apoptosis in MMTV-PyMT OVA+ organoids (FIG. 5J). Importantly, we observed that this cytotoxicity was further enhanced when OT I T cells were pre-treated with docetaxel (FIG. 5J), demonstrating that docetaxel treatment has the potential to enhance T cell killing capacity also in tumor-specific T cells.

This previous assay confirms that taxanes can induce T cell mediated killing not only in naïve T cells but also in T cells that have a specific TCR against target cells, which means that taxanes can enhance T cell killing also when there is a TCR signaling going on.

### Vinblastine, an Exemplary Vinca Alkaloid, Induces T Cell Mediated Cancer Cell Killing.

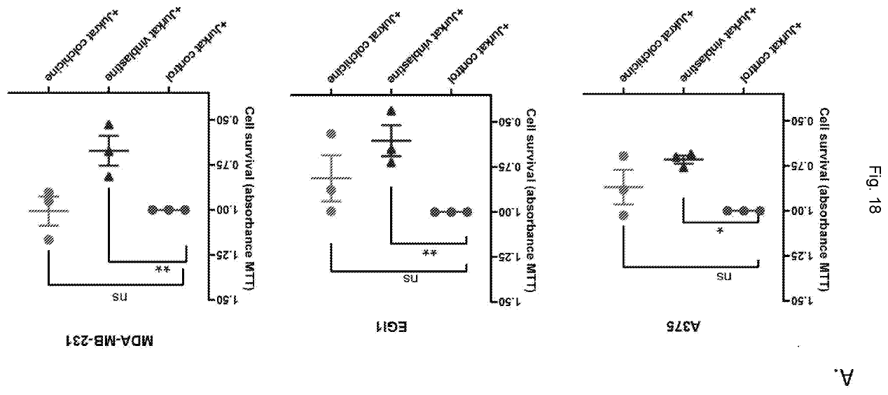

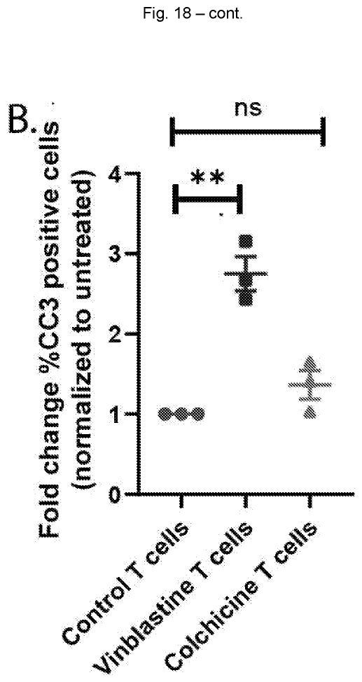

FIG. 18 shows cancer cell survival (assessed by MTT assay) following co-culture with human Jurkat T cells pretreated with control, vinblastine, or colchicine. Data are shown for three different cell lines derived from human cancer, namely A375 (melanoma), EGL1 (hepatobiliary cancer) and MDA-MB-231 (breast cancer). Organoid apoptosis (determined by cleaved caspase 3 staining) in breast cancer mouse KB1P organoids following co-culture with mouse splenic T cells pretreated with control, vinblastine or colchicine is also shown in FIG. 18. Data are presented as mean with SEM. N=3 biological repeats per repeat. Vinblastine, an exemplary vinca alkaloid, induces T cell mediated cancer cell killing. This data shows that also the antimitotic agents vinca alkaloids, exemplified by vinblastine, induce T cell mediated cancer killing like the taxanes. This suggests that, like taxanes, vinca alkaloids may be used within the context of the invention as disclosed herein, similar to the taxanes.

### Discussion

For decades, cancer patients have been treated with antimitotic agents, in particular taxanes and/or vinca alkaloids, however their efficacy remains sub-optimal in part due to the administration of low doses to manage toxicity. The mode of action of antimitotic agents, in particular taxanes and/or vinca alkaloid in vivo has been contentious, which hinders the design of optimal treatment regimens. Although, for example, taxanes induce tumor cell death through mitotic perturbations in vitro, they appear to exert their anti-tumor effects in in vivo cancer models and in cancer patients through other means.

We surprisingly found that antimitotic agents, in particular taxanes and/or vinca alkaloids, exemplified by, for example, docetaxel, can activate T cells to directly kill cancer cells. We reveal that the agents directly increase the cytotoxicity of both tumor-naïve and tumor-reactive T cells in a TCR-independent, non-classical manner.

Additionally, we demonstrate that docetaxel triggers T cells to release cytotoxic extracellular vesicles, which in turn eliminate tumor cells. Moreover, in the context of immunodeficient mouse models of cancer which respond to treatment with taxanes, although T cells cannot mature in a canonical way, our data suggest that they can be stimulated upon docetaxel treatment, independently of TCR signaling. This establishes a novel paradigm regarding the mode of action of antimitotic agents, in particular taxanes and/or vinca alkaloids in vivo.

EVs are emerging as critical drivers of cancer progression, metastasis, and response to treatments. A large amount of work has focused on the roles of EVs derived from cancer cells, however, EVs can also be released by a number of immune cell populations, such as NK cells and cytotoxic T cells (CTLs). CTL-released EVs have been shown to either promote metastasis or block invasion, to alter dendritic cell and B cell activity and to influence naïve T cells. Here, we demonstrate that EVs released by T cells upon treatment with docetaxel have cytotoxic capacities against tumor cells but not against their healthy surrounding counterparts. T cell-mediated killing of target cells is classically thought to be supported by engagement of the TCR upon specific recognition of an antigen. In contrast, antimitotic, in particular taxanes and/or vinca alkaloids induced release of cytotoxic EVs, such as docetaxel-induced release of cytotoxic EVs is TCR-independent.

The preferential killing of tumor cells over healthy cells can be explained by the higher capacity of tumor cells to uptake T cell-derived EVs.

Docetaxel's microtubule-stabilization activity may explain the differential release of cytotoxic EVs by T cells upon treatment with docetaxel. Indeed, upon stimulation of the TCR, T cells translocate their microtubule organizing center to the contact site in order to polarize cytokine secretion and release of cytotoxic vesicles. Microtubules likewise support the trafficking and release of cytokines and vesicles. Consequently, it is possible that by stabilizing microtubules, docetaxel can bypass TCR activation to allow direct, untargeted release of cytotoxic EVs in the surrounding T cells environment.

Leveraging the interactions between the immune system and anti-cancer therapies has led to important improvements in patient care in a variety of cancer types. The majority of these approaches focuses on altering the immune properties of cancer cells to activate TCR signaling, and tumor-reactive immune cells mediated killing (Kersten, K., C. Salvagno, and K. E. de Visser, Front Immunol, 2015. 6: p. 516; Zitvogel, L., et al., Nat Rev Immunol, 2008. 8(1): p. 59-73; Galluzzi, L., et al. Nat Rev Immunol, 2017. 17(2): p. 97-111; Voorwerk, L., et al. Nat Med, 2019. 25(6): p. 920-928). Here, a striking aspect of our findings is that taxanes-induced T cell responses are independent of the TCR, therefore occurs also in tumor-naïve T cells that have never been educated by cancer cells.

Our study allows for the development of new TCR-independent anti-cancer immunotherapies with a broad impact, for instance in solid cold tumors in which neoantigens are rare. Collectively, our findings suggest that transfer of T cells pre-treated with antimitotic agents, in particular taxanes and/or vinca alkaloids, or EVs secreted by such T cells, can reduce tumor burden without systemic exposure to antimitotic agent toxicity, in particular taxanes and/or vinca alkaloids-associated toxicity (neuropathy) and has the potential to increase antimitotic agent, in particular taxanes and/or vinca alkaloids, efficacy while reducing their side effects.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1.-14. (canceled)

15. Method of treatment of a subject in need thereof wherein the method comprises administering to the subject T cells obtained by contacting isolated T cells with at least one antimitotic agent and/or administering to the subject extracellular vesicles released from said T cells.

16. Method of treatment according to claim 15 wherein the T cells and/or extracellular vesicles are obtained by:
(i) providing isolated T cells;
(ii) contacting the provided T cells with at least one antimitotic agent, preferably selected from the groups consisting of a taxane and a vinca alkaloid; and
(iii) obtaining the T cells contacted with the at least one antimitotic agent, and/or obtaining the extracellular vesicles released from said T cells.

17. Method of treatment according to claim 15 wherein the subject is a cancer subject and/or wherein the treatment is in order to prevent neuropathy, vinca alkaloid-induced neuropathy and/or taxane-induced neuropathy in a subject.

18.-19. (canceled)

20. Method of treatment according to claim 15 wherein the method comprises administering the T cells to the subject.

21. Method of treatment according to claim 15 wherein the method comprises administering the extracellular vesicles to the subject.

22. Method of treatment according to claim 15 wherein the T cells comprise CD4+ T cells and/or CD8+ T cells.

23. Method of treatment according to claim 15 wherein the T cells are autologous T cells, allogenic T cells or syngeneic T cells.

24. Method of treatment according to claim 15 wherein the at least one antimitotic agent is a vinca alkaloid.

25. Method of treatment according to claim 24 wherein the vinca alkaloid is selected from the group consisting of vinblastine, vinorelbine, vincristine, vindesine and vinflunine.

26. Method of treatment according to claim 15 wherein the at least one antimitotic agent is a taxane.

27. Method of treatment according to claim 26 wherein the taxane is selected from the groups consisting of paclitaxel and docetaxel.

28. Method of treatment according to claim 15 wherein the subject is a cancer subject, wherein the cancer is selected from the group consisting of breast cancer, lung cancer, ovarian cancer, bladder cancer, prostate cancer, a sarcoma, a carcinoma, esophageal cancer, melanoma, glioma, glioblastoma, liver cancer, colon cancer, rectal cancer, leukemia, lymphoma, Hodgkin's disease, multiple myelomas, and cholangiocarcinoma.

29. Method of treatment according to claim 15 wherein the subject is selected from the group consisting of a human, a male, a female, a child, a human that is allergic to an antimitotic agent, preferably selected from the groups consisting of a taxane and a vinca alkaloid, a human that elicits a hypersensitive reaction to an antimitotic agent, preferably selected from the groups consisting of a taxane and a vinca alkaloid taxane, and/or a human that develops a neuropathy during systemic treatment with an antimitotic agent, preferably selected from the groups consisting of a taxane and a vinca alkaloid.

30. Method of producing treated T cells, and/or extracellular vesicles released from said T cells, wherein the method comprises the steps of
(a) providing isolated T cells;
(b) contacting the provided T cells with at least one antimitotic agent selected from the group consisting of a vinca alkaloid and a taxane; and
(c) obtaining the T cells contacted with at least one antimitotic agent, and/or obtaining the extracellular vesicles released from said T cells, to provide for the treated T cells and/or extracellular vesicles released from said T cells.

31. The method according to claim 30 wherein the method is for producing a medicament and wherein step (c) comprises obtaining the T cells contacted with the at least one antimitotic agent, and/or obtaining the extracellular vesicles released from said T cells, to provide for the medicament.

32. The method according to claim 30 wherein
(i) in step (b) the provided T cells are contacted with at least one taxane, wherein the taxane concentration is at least 0.01 nmol/L, preferably between 0.1 nmol/L and 500 nmol/L;
(ii) in step (b) the provided T cells are contacted with the antimitotic agent for a period of at least 1 hour, preferably between 6 hours and 200 hours;
(iii) in step (b) the provided T cells are contacted with the antimitotic agent at a temperature of at least 14 degrees Celsius, preferably between 14 degrees Celsius and 45 degrees Celsius;
(iv) in step (c) T cells are obtained by isolating the T cells from the medium in which the contacting of step (b) is performed; and/or
(v) in step (c) the extracellular vesicles released from the T cells are obtained by isolating the extracellular vesicles released from the T cells from the medium in which the contacting of step (b) is performed.

33. Method of treatment according to claim 15 wherein the antimitotic agent is selected from the group consisting of a taxane and a vinca alkaloid.
